(19) United States
(12) Patent Application Publication
(10) Pub. No.: US 2005/0054822 A1
(43) Pub. Date: Mar. 10, 2005

(54) Hin-2 nucleic acid molecules, proteins, antibodies, homologues, receptors, and uses thereof

(76) Inventors: Hong-Bing Shu, Denver, CO (US); Liang Hua Bin, Denver, CO (US)

Correspondence Address: SHERIDAN ROSS PC, 1560 BROADWAY, SUITE 1200, DENVER, CO, 80202, US

(21) Appl. No.: 10/492,717

(22) PCT Filed: Oct. 21, 2002
(86) PCT No.: PCT/US02/33785

Related U.S. Application Data
(60) Provisional application No. 60/343,121, filed on Oct. 19, 2001.

(51) Int. Cl. C07H 21/04; A61K 39/00; C07K 14/74

(52) U.S. Cl. 530/350; 435/69.1; 435/320.1; 435/325; 424/192.1; 536/23.2

(57) ABSTRACT

Disclosed are HIN-2 proteins and homologues, nucleic acid molecules encoding HIN-2 proteins and homologues, antibodies that selectively bind to HIN-2 proteins and homologues; compositions comprising HIN-2 proteins and homologues, nucleic acid molecules, or antibodies; and methods of making and using HIN-2 proteins and homologues, nucleic acid molecules, and antibodies. Also disclosed are receptors and ligands that selectively bind to HIN-2, as well as fragments and homologues of such receptors, agonists and antagonists of such receptors, and methods of using such receptors to regulate the biological activity mediated by HIN-2.

### FIELD OF THE INVENTION

The present invention relates to HIN-2 proteins and homologues, to nucleic acid molecules encoding such proteins and homologues, to antibodies that selectively bind to HIN-2 proteins, to HIN-2 receptors, and to methods of making and using these agents.

### BACKGROUND OF THE INVENTION

Adaptive immune recognition relies on the generation of a random and highly diverse repertoire of antigen receptors (T and B lymphocyte receptors), followed by clonal expansion and selection of receptors with relevant specificities. This mechanism accounts for the generation of immunological memory, which provides a significant adaptive fitness to vertebrate animals. However, the adaptive immune response has two main limitations. First, randomly generated antigen receptors are unable to determine the source and the biological context of the antigen for which they are specific. Second, a clonal distribution of antigen receptors requires that specific clones expand and differentiate into effector cells before they can contribute to host defense. As a result, primary adaptive immune responses are delayed, typically for about 4-7 days, which is too much of a delay to combat quickly replicating microbial invaders. Fortunately, vertebrates have a first line of defense provided by a evolutionarily ancient and more universal innate immune system. Indeed, many aspects of the adaptive immune response are controlled by a combination of permissive and instructive signals that are provided by the innate immune system. The innate immune system detects the presence and the nature of an infection or other foreign presence in a host, provides the first line of host defense, and controls the initiation and determination of the effector class of the adaptive immune response. (Peiser et al., 2002, Curr. Opin. Immunol. 14:123-128)

Innate immune responses depend on the detection and recognition of constitutive and conserved products of microbial metabolism. For example, lipopolysaccharide (LPS) and lipoproteins are examples of molecules produced by bacteria but not eukaryotes and therefore are signatures of microbial invaders. The principal functions of the innate immune system are opsonization, activation of complement and coagulation cascades, phagocytosis, activation of proinflammatory signaling pathways, and induction of apoptosis. Innate immune recognition is not antigen-specific, but there are patterns which are associated with different types of microbes to which the receptors of the innate immune system can respond. Additionally, the innate immune system has the ability to generally distinguish between microbes that are ubiquitous in a host and those that are invaders, such as by using compartmentalization and anti-inflammatory cytokines. (Peiser et al., 2002, Curr. Opin. Immunol. 14:123-128)

Inflammation is generally characterized by the influx of certain cell types and mediators, the presence of which can lead to amplification of a specific type of immune response, microbe clearance, and frequently to tissue damage and sometimes death. The innate immune response induces the production of various cytokines which mediate the interaction between the non-specific inflammatory response and the adaptive immune response. The cytokine network is a homeostatic system, in which the activity of one cytokine can be synergized or inhibited by another cytokine. The type of cytokine activity and prevalent adaptive immune response depends on the stage of inflammation, the local environment, and/or the type of injury, infection, or other stimulus and thus, to a large extent, can be initially controlled by the innate immune response. A proinflammatory immune response is typically characterized by the upregulation of cytokines that are considered to be proinflammatory (e.g., tumor necrosis factor-α (TNF-α), interleukin-12 (IL-12), interleukin-1 (IL-1), interferon-γ (IFN-γ), interleukin-6 (IL-6), interleukin-8 ([L-8)). Typically associated with many of these proinflammatory cytokines is the activation of T helper lymphocytes referred to as “Th1” type lymphocytes and the production of antibody isotypes IgG2a or IgG3 (the approximate human equivalent of which is IgG1, IgG2 or IgG3). Th1 type immune responses are most prevalent in response to microbial invasion, including viral and bacterial infection, as well as in autoimmune disease. Another group of cytokines which is typically considered to be anti-inflammatory, because they antagonize the action of some of the above-identified proinflammatory cytokines, include interleukin-4 (IL-4), interleukin-5 (IL-5),interleukin-9 (IL-9), interleukin-10 (IL-10), interleukin-13 (IL-13) and interleukin-15 (IL-15). Cytokines of this type are typically associated with a Th2 response, in which “Th2” type lymphocytes and production of antibody isotypes IgG1 (the approximate human equivalent of which is IgG4) and IgE prevail. However, Th2 type immune response can also be considered to be inflammatory under certain conditions, as this is the prevalent immune response in allergic inflammation and in the response to parasitic infections. Importantly, elicitation of a Th1-type response in an animal that is undergoing a Th2-type response, or vice versa, may change the overall effect of the immune response from harmful to beneficial. Therefore, the control of inflammation and the immune response in any given infection, condition or disease is likely to be dependent on a fine regulation of the various non-antigen-specific and antigen-specific variables in the host which drive the inflammatory response.

Therefore, there is a continued need for new reagents and methods to control inflammation and regulate the innate and adaptive immune response to various infectious and non-infectious agents that direct the course of the host defense.

### SUMMARY OF THE INVENTION

One embodiment of the present invention relates to an isolated protein comprising an amino acid sequence selected from the group consisting of: (a) an amino acid sequence selected from the group consisting of SEQ ID NO:2, amino acids 22-93 of SEQ ID NO:2, SEQ ID NO:4, and amino acids 22-91 of SEQ ID NO:4; (b) an amino acid sequence that is at least about 50% identical to the amino acid sequence of (a), wherein the protein has HIN-2 biological activity; and, (c) an amino acid sequence consisting of a fragment of an amino acid sequence of (a) that has HIN-2 biological activity. In another embodiment, the protein comprises an amino acid sequence that is at least about 75% identical to the amino acid sequence of (a), and in another embodiment, at least about 85% identical to the amino acid sequence of (a), and in another embodiment, at least about 95% identical to the amino acid sequence of (a). Fusion proteins comprising any of such proteins and compositions comprising any of such proteins are also contemplated.

Another embodiment of the invention relates to a human HIN-2 homologue, wherein the human HIN-2 homologue comprises an amino acid sequence that is at least about 50% identical to SEQ ID NO:2 or amino acids 22-93 of SEQ ID NO:2, and less than 100% identical to an amino acid sequence selected from the group consisting of SEQ ID NO:2, amino acids 22-93 of SEQ ID NO:2, SEQ ID NO:4, and amino acids 22-91 of SEQ ID NO:4. The human HIN-2 homologue is an agonist or antagonist of HIN-2 biological activity. In one embodiment, the HIN-2 homologue comprises an amino acid sequence that is less than about 97% identical to an amino acid sequence selected from the group consisting of SEQ ID NO:2, amino acids 22-93 of SEQ ID NO:2, SEQ ID NO:4, and amino acids 22-91 of SEQ ID NO:4, and in another embodiment, less than about 95% identical to such amino acid sequence, and in another embodiment, less than about 90% identical to such amino acid sequence. In one aspect, the HIN-2 homologue comprises an amino acid sequence that is at least about 75% identical to SEQ ID NO:2 or amino acids 22-93 of SEQ ID NO:2, and in another embodiment, at least about 85% identical to SEQ ID NO:2 or amino acids 22-93 of SEQ ID NO:2, and in another embodiment, at least about 95% identical to SEQ ID NO:2 or amino acids 22-93 of SEQ ID NO:2. In one aspect, any of such human HIN-2 homologues is an agonist of HIN-2 biological activity. In another aspect, any of such human HIN-2 homologues is an antagonist of HIN-2 biological activity. In another aspect, any of such human HIN-2 homologues binds to a HIN-2 receptor (e.g., MARCO). In another aspect, any of such human HIN-2 homologues binds to a bacterium or to a yeast. In another aspect, any of such human HIN-2 homologues binds to lipopolysaccharide (LPS). In yet another aspect, any of such human HIN-2 homologues binds to apolipoprotein A1. In yet another aspect, any of such human HIN-2 homologues regulates phagocytosis of bacteria and bacterial components or yeast and yeast components by macrophages. In another aspect, any of such human HIN-2 homologues regulates allergic inflammation in the lung of a mammal. In another aspect, any of such human HIN-2 homologues reduces inflammation in the lung of a mammal. In yet another aspect, any of such human HIN-2 homologues regulates the production of proinflammatory cytokines. In another aspect, any of such human HIN-2 homologues regulates the development of allergic airway hyperresponsiveness in a mammal. Also included in the invention are fusion proteins comprising any of such human HIN-2 homologues or compositions comprising any of such HIN-2 homologues.

Another embodiment of the present invention relates to an isolated nucleic acid molecule comprising a nucleic acid sequence encoding of the isolated proteins and/or human HIN-2 homologues set forth above. In one aspect, the nucleic acid sequence comprises SEQ ID NO:1 or SEQ ID NO:3. Another embodiment relates to a recombinant nucleic acid molecule comprising any of such isolated nucleic acid molecules, operatively linked to a transcription control sequence. Yet another embodiment relates to a recombinant host cell that has been transfected with such a recombinant nucleic acid molecule. Another embodiment relates to a method of producing a protein comprising culturing the recombinant host cell under conditions whereby the protein encoded by the recombinant nucleic acid molecule is produced. Also included is a recombinant virus comprising any of the above-identified isolated nucleic acid molecules. Another aspect relates to a composition comprising any of the above-identified isolated nucleic acid molecules.

Another embodiment relates to an oligonucleotide consisting of at least 13 consecutive nucleotides, and in another embodiment, at least about 25 consecutive nucleotides, and in another embodiment, at least about 50 consecutive nucleotides, and in another embodiment, at least about 100 consecutive nucleotides, of a nucleic acid sequence selected from the group consisting of SEQ ID NO:1 and SEQ ID NO:3. In one aspect, the oligonucleotide is immobilized on a substrate. In another aspect, a plurality of oligonucleotides is immobilized on a substrate.

Another embodiment of the invention relates to an isolated binding agent selected from an antibody, an antigen binding fragment and a binding partner, wherein the binding agent selectively binds to an amino acid sequence selected from the group consisting of SEQ ID NO:2, amino acids 22-93 of SEQ ID NO:2, SEQ ID NO:4, and amino acids 22-91 of SEQ ID NO:4. In one aspect, the binding agent is immobilized on a substrate. In another aspect, the invention includes a composition comprising the binding agent.

Yet another embodiment of the present invention relates to a method to identify a compound that regulates HIN-2 expression or biological activity. The method includes the steps of (a) contacting a HIN-2 protein, a biologically active fragment thereof, or a receptor-binding fragment thereof, with a putative regulatory compound; and (b) detecting whether the putative regulatory compound binds to or regulates the activity of the HIN-2 protein, biologically active fragment or receptor-binding fragment as compared to prior to contact with the compound. A compound that binds to the protein and increases or decreases activity of the protein, as compared to the protein in the absence of the compound, indicates that the putative regulatory compound is a regulator of HIN-2. In one aspect, the step of detecting comprises detecting whether HIN-2 expression or activity is regulated by contacting lung cells or lung tissue with the putative regulatory compound and measuring HIN-2 expression in the lung cells or lung tissue. In another aspect, the step of detecting comprises detecting whether HIN-2 expression or activity is regulated during allergic inflammation conditions in the presence of the putative regulatory compound as compared to in the absence of the putative regulatory compound. In another aspect, the step of detecting comprises detecting whether HIN-2 expression or activity is regulated during proinflammatory conditions in the presence of the putative regulatory compound as compared to in the absence of the putative regulatory compound. In yet another embodiment, the step of detecting comprises detecting whether the putative regulatory compound inhibits, enhances, or competes with the binding of HIN-2 to a HIN-2 receptor (e.g., MARCO). In another embodiment, the step of detecting comprises detecting whether the putative regulatory compound inhibits, enhances, or competes with the binding of HIN-2 to a ligand selected from the group consisting of lipopolysaccharide (LPS), apolipoprotein AI, a bacterium, or a yeast. In another embodiment, the method further comprises a step of detecting whether the putative regulatory compound regulates inflammation in a cell, tissue, or non-human animal (e.g., in the lung cells or lung tissue of the animal). In another embodiment, the method further comprises a step of detecting whether the putative regulatory compound regulates the level of high density lipoproteins or low density lipoproteins in a mammal.

Yet another embodiment of the present invention relates to a method to identify a HIN-2 homologue that regulates HIN-2 biological activity, comprising detecting whether a putative HIN-2 homologue has at least one biological activity selected from the group consisting of: (a) binds to a HIN-2 receptor or to a HIN-2-binding portion of a HIN-2 receptor; (b) increases the activity of a HIN-2 receptor; (c) binds to a bacterial cell; (d) binds to a yeast; (e) binds to a lipopolysaccharide (LPS); (f) binds to apolipoprotein Al; (g) regulates phagocytosis of a bacterium by a macrophage as compared to in the absence of the putative HIN-2 homologue; (h) regulates lung inflammation as compared to in the absence of the putative HIN-2 homologue; (i) regulates airway hyperresponsiveness as compared to in the absence of the putative HIN-2 homologue; or (j) regulates innate immune responses in lung tissue as compared to in the absence of the putative HIN-2 homologue. In one aspect, the method comprises contacting a HIN-2 receptor (e.g., MARCO) or a HIN-2-binding portion of a HIN-2 receptor with the putative HIN-2 homologue, and detecting whether the putative HIN-2 homologue binds to the HIN-2 receptor or HIN-2-binding portion. In one embodiment, the putative HIN-2 homologue comprises an amino acid sequence that is at least about 50% identical to SEQ ID NO:2 or amino acids 22-93 of SEQ ID NO:2, and less than 100% identical to an amino acid sequence selected from the group consisting of SEQ ID NO:2, amino acids 22-93 of SEQ ID NO:2, SEQ ID NO:4, and amino acids 22-91 of SEQ ID NO:4. In another embodiment, the HIN-2 homologue comprises an amino acid sequence that is less than about 97% identical to an amino acid sequence selected from the group consisting of SEQ ID NO:2, amino acids 22-93 of SEQ ID NO:2, SEQ ID NO:4, and amino acids 22-91 of SEQ ID NO:4. In yet another embodiment, the HIN-2 homologue comprises an amino acid sequence that is less than about 95% identical to an amino acid sequence selected from the group consisting of SEQ ID NO:2, amino acids 22-93 of SEQ ID NO:2, SEQ ID NO:4, and amino acids 22-91 of SEQ ID NO:4. In yet another embodiment, the HIN-2 homologue comprises an amino acid sequence that is at least about 75% identical to SEQ ID NO:2 or amino acids 22-93 of SEQ ID NO:2. In yet another embodiment, the HIN-2 homologue comprises an amino acid sequence that is at least about 95% identical to SEQ ID NO:2 or amino acids 22-93 of SEQ ID NO:2.

Yet another embodiment of the present invention relates to a method to identify a compound that regulates the expression of Hin-2, comprising: (a) contacting a putative regulatory compound with a recombinant host cell that expresses a recombinant nucleic acid molecule encoding HIN-2 or a recombinant host cell that has been transfected with a nucleic acid sequence comprising a Hin-2 regulatory region operatively linked to a reporter nucleic acid sequence; and (b) detecting whether the putative regulatory compound regulates expression of the recombinant nucleic acid molecule encoding HIN-2 or the reporter nucleic acid sequence as compared to prior to contact with the compound. Compounds that regulate expression of the recombinant nucleic acid molecule encoding HIN-2 or the reporter nucleic acid sequence, as compared to in the absence of the compound, indicates that the putative regulatory compound is a regulator of Hin-2 expression.

Another embodiment of the invention relates to a method to diagnose a disorder associated with HIN-2 expression or biological activity, comprising detecting expression or biological activity of HIN-2 or a gene encoding HIN-2 in a tissue of a patient suspected of having the disorder, and comparing the expression or biological activity to a control, wherein a difference in the expression or biological activity of HIN-2 or a gene encoding HIN-2 in a tissue of the patient as compared to the control indicates a positive diagnosis of a disorder associated with HIN-2. In one aspect, the disorder is a lung disorder. In another aspect, the disorder is associated with allergic inflammation. In another aspect, the disorder is associated with microbial infection. In another aspect, the disorder is selected from the group consisting of asthma, interstitial lung disease, cystic fibrosis, rheumatoid arthritis, reactive arthritis, bacterial infection, yeast infection, and spondylarthropathy. In another aspect, the disorder is a lung cancer.

Yet another embodiment of the present invention relates to a composition comprising a portion of MARCO sufficient to bind to HIN-2 that is formulated for administration to lung tissue. In one embodiment, the MARCO is a soluble receptor.

Another embodiment of the present invention relates to a method to regulate HIN-2 expression or activity, comprising administering to a patient a regulatory compound that regulates the biological activity of HIN-2. In one aspect, the compound is HIN-2 or a biologically active fragment thereof. In another aspect, the compound is a HIN-2 homologue, including, but not limited to, a HIN-2 homologue that binds to MARCO and regulates phagocytosis of bacteria or yeast by a macrophage, and a HIN-2 homologue that binds to MARCO and induces NFκB activation in a cell expressing MARCO. In another embodiment, the regulatory compound is an antibody, antigen binding fragment or binding partner that selectively binds to HIN-2. In another aspect, the regulatory compound is an antibody, an antigen binding fragment or a binding partner that selectively binds to a HIN-2 receptor (e.g., MARCO). In another embodiment, the regulatory compound is a HIN-2 receptor homologue that selectively binds to HEN-2, such as a soluble HIN-2 receptor, or a homologue of MARCO. In another embodiment, the regulatory compound is an isolated nucleic acid sequence that hybridizes to a nucleic acid sequence encoding at least 13 consecutive nucleotides of a gene encoding HIN-2. In another embodiment, the regulatory compound regulates the ability of HIN-2 to bind to a HIN-2 receptor. In yet another embodiment, the regulatory compound regulates the biological activity of HIN-2. In another embodiment, the regulatory compound regulates the expression of HIN-2. In another embodiment, the patient has or is at risk of developing an inflammatory condition in the lung. In another embodiment, the patient has or is at risk of developing an inflammatory condition in the joints. In another embodiment, the patient has or is at risk of developing an autoimmune disease. In another embodiment, the patient has or is at risk of developing atherosclerosis. In another embodiment, the patient has or is at risk of developing lung cancer.

Yet another embodiment of the present invention relates to a method to regulate inflammation, comprising administering to a patient a compound that regulates the expression or biological activity of HIN-2 or a gene encoding HIN-2 in the patient. In one aspect, the inflammation is in the lung of the patient. In another aspect, the inflammation is allergic inflammation. In another embodiment, the inflammation is associated with a microbial infection. In one aspect, the patient has a condition selected from: asthma, interstitial lung disease, cystic fibrosis, rheumatoid arthritis, reactive arthritis, lung cancer, bacterial infection, yeast infection and spondylarthropathy.

Another embodiment of the invention relates to a method to regulate the levels of high density lipoproteins or low density lipoproteins in a patient, comprising administering to a patient a regulatory compound that binds to HIN-2 and reduces binding of HIN-2 to MARCO or to apolipoprotein AI.

Yet another embodiment of the invention relates to a method to treat cancer in a patient, wherein the tumor cells of the patient express MARCO, the method comprising administering to a patient with cancer a regulatory agent comprising a portion of HIN-2 sufficient to bind to MARCO which is linked to a therapeutic compound which reduces tumor cell growth or eliminates the tumor cell. In one aspect, the cancer is a lung cancer and the tumor cells are lung tumor cells.

Another embodiment of the present invention relates to a method to deliver a therapeutic compound to a macrophage which expresses MARCO in a patient, comprising administering to the patient a regulatory agent comprising a portion of HIN-2 sufficient to bind to MARCO which is linked to the therapeutic compound.

Yet another embodiment of the invention relates to a genetically modified non-human animal comprising a genetic modification within at least one allele of its Hin-2 locus, wherein the genetic modification results in a reduction of HIN-2 biological activity in the animal. In one aspect, the animal comprises a genetic modification in both alleles of its Hin-2 locus, wherein the genetic modification results in an absence of HIN-2 biological activity in the animal.

Another embodiment of the invention relates to a method to detect a polymorphism or a loss of heterozygosity located in chromosome region 5q30-40, comprising contacting a nucleic acid molecule from a patient with an oligonucleotide comprising at least 13 consecutive nucleotides of SEQ ID NO:1.

### BRIEF DESCRIPTION OF THE DRAWINGS OF THE INVENTION

FIG. 1A is a digitized image of a Western blot showing that HIN-2 is an about 10 kD secreted protein.

FIG. 1B is a digitized image Western blot showing the purification of HIN-2 by affinity column.

FIG. 2 is a digitized image of a Northern blot showing the tissue distribution of HIN-2.

FIG. 3A is a digitized image of a Northern blot showing the tissue distribution of MARCO.

FIG. 3B is a digitized image of a Northern blot showing the expression of MARCO in monocytes.

FIG. 3C is a digitized image of a Northern blot showing the expression of MARCO in lung cancer cell lines.

FIG. 4A is a digitized image of a Western blot showing that HIN-2 binds to viable and non-viable, Gram positive and Gram negative bacteria, and to viable and non-viable yeast.

FIG. 4B digitized image of a Western blot showing that HIN-2 binds to LPS and that LPS inhibits the binding of HIN-2 to bacteria.

FIG. 5 is a bar graph showing that overexpression of MARCO activates NFκB.

### DETAILED DESCRIPTION OF THE INVENTION

The present invention generally relates to the discovery of a novel protein, referred to herein as HIN-2; to nucleic acid molecules encoding such proteins and homologues; to antibodies that selectively bind to such proteins; to compositions comprising such proteins, nucleic acid molecules, and antibodies; and to methods of making and using such protein, nucleic acid molecules, and antibodies. Both human and mouse HIN-2 are disclosed herein. Also encompassed by the present invention are homologues of HIN-2 and of nucleic acid sequences encoding HIN-2 (e.g., including agonists or antagonists of HIN-2 and HIN-2 receptors), as well as peptide or synthetic mimetics of HIN-2, its receptor, or nucleic acid sequences encoding HIN-2. Also encompassed by the present invention are receptors that selectively bind to HIN-2, as well as fragments and homologues of such receptors, agonists and antagonists of such receptors, and methods of using such receptors to regulate the biological activity mediated by HIN-2.

The present inventors have identified and cloned a protein (represented by SEQ ID NO:2) that they initially determined was approximately 43% identical (84% homologous) to the amino acid sequence of another recently identified human protein called HIN-1 (high in normal-1) (GenBank Accession No. AAK82942.1) (Krop et al., 2001, Proc. Natl. Acad. Sci. USA 98(17):9796-9801). HIN-1 is a putative cytokine that is expressed in normal mammary epithelial cells, and which is also highly expressed in the lung. HIN-1 is also found at low levels in the stomach, heart, small intestine, and uterine tissue (Porter et al., 2002, Mech. Dev. 114:201). HIN-1 been described as a candidate tumor suppressor gene because it is significantly downregulated in 94% of human breast carcinomas and in 95% of preinvasive lesions (Krop et al., 2001, supra). This inactivation in the earliest stages of breast tumorigenesis) accompanied by hypermethylation of its promoter in the majority of breast cancer cell lines and primary tumors, and reintroduction of HIN-1 into breast cancer cells inhibits cell growth (Krop et al., 2001, supra). Hin-1 is located on chromosome 5 (5q35-tel). HIN-1 has also been subsequently referred to as UGRP2 (Niimi et al., November 2001, Mol. Endocrinol. 15(11):2021-2036).

The protein identified by the present inventors, like HIN-1, is encoded by a gene that is located on chromosome 5 (5q30-40 region), and was shown by the inventors to be primarily expressed in the lung tissue (See Examples). The present inventors have also shown that HIN-2 is a secreted protein (a putative cytokine) and that HIN-2 binds to a receptor (e.g., a HIN-2 receptor) on at least two human lung cancer cell lines (See Examples) (see also the priority document U.S. Provisional Application Ser. No. 60/343,121, which is incorporated herein by this reference in its entirety). In addition, as discussed in detail below, the present inventors have discovered the identity of a receptor for HIN-2, as well as other binding ligands for HIN-2, all of which provide information regarding the function of HIN-2. In view of the sequence identity and other characteristics shared between the identified protein and HIN-1 (and therefore the possibility that the proteins might have at least some related functions), the present inventors named the newly discovered protein HIN-2 (encoded by a gene referred to herein as Hin-2). The present inventors have additionally proposed that HIN-2 plays a particular role in the lung and has an effect in lung conditions (e.g., asthma, interstitial lung disease, etc.). The present inventors have in fact found that expression of HIN-2 is upregulated in the lung tissue of patients with cystic fibrosis as compared to normal patient controls (See Examples). Moreover, given the homology of HIN-2 to HIN-1 and binding of HIN-2 to a receptor on at least two human lung cancer cell lines, without being bound by theory, the present inventors have also suggested the possibility that HIN-2 may also have an effect in lung cancer (e.g., as a tumor suppressor or other tumor regulator).

The sequence identified by the present inventor was previously presented as part of several different expressed sequence tag clones from human sequences (e.g., Accession Nos. BG545709, AI734244, BG537963). None of these EST clone submissions disclosed a coding sequence within the clone for a putative protein, nor did any of these submissions propose any function for any protein that might be located within the clone. Therefore, the EST clone submissions did not identify a HIN-2 protein or even the specific coding sequence therefor.

The 93-amino acid, human HIN-2 sequence described herein (represented by SEQ ID NO:2) is approximately 43% identical to the human HIN-1 amino acid sequence (GenBank Accession No. AAK82942.1) over the full length of the human HIN-2 sequence. HIN-2 was also described in the priority document for this application as being approximately 44% identical to a protein in the public database called “human hypothetical protein XP_{—}044113” over about 83 amino acids of human HIN-2. The XP_{—}044113 protein is now a retracted entry and appears to be an incomplete version of HIN-1. The cDNA sequence containing the human Hin-2 coding region is represented herein as SEQ ID NO:1. Nucleotides 95-373 of SEQ ID NO:1 (not including the stop codon) encode the complete amino acid sequence for human HIN-2 preprotein (SEQ ID NO:2). The mature human HIN-2 protein consists of amino acids 22-93 of SEQ ID NO:2 (amino acids 1-21 are a signal sequence present on the preprotein). Nucleotides 158-373 (not including the stop codon) of SEQ ID NO:1 encode the mature human HIN-2 protein (amino acids 22-93 of SEQ ID NO:2).

The mouse HIN-2 amino acid sequence described herein (represented by SEQ ID NO:4) is approximately 79% identical to human HIN-2 (SEQ ID NO:2), and is also approximately 35% identical to the human HIN-1 amino acid sequence over the full length of the mouse HIN-2 sequence. Mouse HIN-2 was also described in the priority document to be approximately 36% identical to the human hypothetical protein XP_{—}044113 over about 81 amino acids of mouse HIN-2 (XP_{—}044113 is discussed above). The cDNA sequence containing the mouse Hin-2 coding region is represented herein as SEQ ID NO:3. Nucleotides 84-356 of SEQ ID NO:3 (not including the stop codon) encode the complete amino acid sequence for mouse HIN-2 preprotein (SEQ ID NO:4). The mature mouse HIN-2 protein consists of amino acids 22-91 of SEQ ID NO:4 (amino acids 1-21 are a signal sequence present in the preprotein). Nucleotides 147-356 of SEQ ID NO:3 encode the mature mouse HIN-2 protein (amino acids 22-91 of SEQ ID NO:4).

Subsequent to the filing of the priority document for this application, a publication by Niimi et al. described a gene called Ugrp1 (uteroglobin-related protein 1) which was identified by subtractive hybridization experiments using lung mRNA from T/ebp/Nkx2.1-null mouse embryos and wild-type mouse embryos, and which was located on chromosome 5q31-34 (Niimi et al., November 2001, Mol. Endocrinol. 15(11):2021-2036). The expressed product of the mouse Ugrp1 gene was called UGRP1. Niimi et al. used the mouse Ugrp1 to identify ESTs in the human database and then clone the human Ugrp1, the product of which (human UGRP1) has an amino acid sequence that is identical to the human HIN-2 sequence identified by the present inventors. The mouse UGRP1 of Niimi et al. also has an amino acid sequence that is identical to the mouse HIN-2 amino acid sequence described herein. Therefore, the UGRP1 protein described by Niimi et al. is HIN-2. While the protein described in the present invention is referred to herein primarily as HIN-2 (encoded by Hin-2), the protein can also be referred to as UGRP1 (encoded by Ugrp1).

UGRP1 was also identified as having an amino acid sequence that is similar to the proteins of the uteroglobulin/clara cell secretory protein (CCSP) family (mouse UGRP1 is reported to be 25% identical to mouse uteroglobin/CCSP), and mRNA encoding UGRP1 was found to be predominantly expressed in the lung Clara cells (Niimi et al., 2001, ibid.). CCSP has been proposed to function as a regulator of inflammation in the lung (anti-inflammatory), such as by inhibiting PLA₂. Niimi et al. showed that, in a mouse model of allergic inflammation of the lung (dominated by a Th2 type immune response), the expression level of UGRP1 decreased as compared to controls, leading Niimi et al. to propose a role for UGRP1 in asthma and allergic lung inflammation (Niimi et al., 2001, ibid.). Subsequent to this study, Niimi et al. additionally showed that, in a case study using asthma patients and healthy control individuals, a polymorphism (−112A allele) in the human UGRP1 gene promoter reduced the transcriptional activity of the promoter and was linked to an increased risk of asthma, leading Niimi et al. to conclude that reduced UGRP1 expression might lead to a predisposition to asthmatic inflammation (Niimi et al., 2002, Am. J. Hum. Genet. 70:718-725). However, the exact role of UGRP1 in the asthmatic lung was not entirely clear from these studies.

The present inventors have made significant discoveries that reveal important information about the biological activity of HIN-2. Specifically, the present inventors have also identified a receptor of HIN-2, as well as binding ligands of HIN-2. Using expression cloning techniques, the present inventors have discovered that the receptor referred to in the art as MARCO (macrophage receptor with a collagenous structure) is a receptor of HIN-2 (see Examples). The nucleic acid sequence for human MARCO can be found in GenBank Accession No. NM_{—}006770 and is represented herein by SEQ ID NO:5. SEQ ID NO:5 encodes an amino acid sequence for human MARCO represented herein by SEQ ID NO:6. The nucleic acid sequence for mouse MARCO can be found in GenBank Accession No. NM_{—}010766 and is represented herein by SEQ ID NO:7. SEQ ID NO:7 encodes an amino acid sequence for mouse MARCO represented herein by SEQ ID NO:8. MARCO is a member of the scavenger receptor (SR) family, and specifically belongs to a class of SR known as SR-A (e.g., for a review see Peiser et al., 2002, Curr. Opin. Immunol. 14:123-128, incorporated herein by reference in its entirety). SR encompass a broad range of molecules involved in receptor-mediated endocytosis of selected polyanionic ligands, including modified low density lipoproteins (LDL). Several of these receptors are involved in phagocytosis of apoptotic cells and bacteria, as well as in cell adhesion, and therefore, SR are an important receptor in innate immunity. MARCO is a distinct type-A SR which has been shown to have a role in host antibacterial defense (e.g., see, Peiser et al., 2002, ibid.; Sankala et al., 2002, J. Biol. Chem. 277:33378-33385; van der Laan et al., 1999, J. Immunol. 162:939-947; Elshourbagy et al., 2000, Eur. J. Biochem. 267:919-926; Seta et al., 2001, Arthritis Rheum. 44:931-939; Sakaguchi et al., 1998, Lab Invest. 78:423-434; Su et al., 2001, J. Leuk. Biol. 69:75-80; each of these references is incorporated by reference in its entirety). Cumulatively, these references have shown that MARCO is a primarily expressed on macrophages and monocytes and binds to viable and non-viable, gram positive and gram negative bacteria, as well as lipopolysaccharide (LPS). MARCO has been shown to be functionally involved in the binding and phagocytosis of bacteria, is upregulated in response to inflammatory conditions (e.g., in the presence of bacterial infections and bacterial components and the inflammation associated therewith), and may also have a role in the presentation of antigen to accessory immune cells. Therefore, MARCO has a role in the innate immune response. In mice, MARCO is normally expressed by a subpopulation of macrophages in the spleen marginal zone and freshly harvested peritoneal populations, but is rapidly induced in most tissue macrophages (e.g., including in lung and liver) by BCG infection, bacterial sepsis, or treatment with bacteria or LPS in vitro. In humans, MARCO is normally (i.e., under normal physiological conditions) expressed in the lung, liver and lymph nodes (see also Examples). MARCO has also been shown to be upregulated in mice and/or humans by noninfectious stimuli, such as particulates, severe exercise, joint inflammation and atherosclerotic plaques. MARCO has been shown to be susceptible to modulation in peripheral blood mononuclear cells from patients with reactive arthritis (ReA), Salmonella infection, other types of spondylarthropathy, and rheumatoid arthritis. In contrast to mouse MARCO, human MARCO does not bind to acetylated LDLs and therefore, the role of human MARCO in atherosclerosis is not presently clear.

The present inventors have confirmed that human MARCO is normally expressed in lung and liver and have additionally demonstrated that MARCO is expressed on some lung tumor cell lines (see Examples). In addition, although the signal transduction pathway of MARCO has not been well characterized, the present inventors demonstrate herein that MARCO activates NFκB through a pathway that is conserved with the IL-1 receptor (IL-1R) and the Toll-like receptor family (see Examples). Toll-like receptors are also important in innate immunity, including in microbial recognition and induction of antimicrobial genes, and in control of adaptive immune responses (including dendritic cell maturation and T helper cell responses) (see, e.g., Medzhitov, 2001, Nature. Rev. 1:135-145).

Finally, the present inventors have discovered that HIN-2, in addition to binding to MARCO, also binds to some of MARCO's other ligands, including gram positive and gram negative bacteria (viable or non-viable) and to LPS. HIN-2 has also been found to bind to non-viable and viable yeast cells. The inventors have also discovered that LPS competes with HIN-2 for binding to MARCO. LPS is a bacterial product and is a proinflammatory mediator (e.g., it induces the production of proinflammatory cytokines such as interleukin-l1 (IL-1,5), interleukin-6 (IL-6), interleukin-8 (IL-8), and tumor necrosis factor-α (INF-α)).

Additionally, the present inventors have discovered that HIN-2 binds to apolipoprotein AI (ApoA-I). ApoA-I is the major apolipoprotein of human high density lipoprotein (HDL) and plays, a significant role in cholesterol transport and atherosclerosis protective). ApoA-I has anti-inflammatory properties, it can bind to and inhibit LPS, it has been shown to bind to macrophages (Burgess et al., 2002, J Biol. Chem. 277:31318-31326), and it has been shown to inhibit the contact-mediated activation of monocytes which bind to stimulated T cells, thus inhibiting TNF-α and IL-1β and inhibits monocyte inflammatory functions in peripheral blood mononuclear cells (Hyka et al., 2001, Blood 97(8):2381-9). Autoantibodies to ApoA-I and HDL have been found in patients with systemic lupus erythematosus (Dinu et al., 1998, Lupus 7(5):355-60; Abe et al., 2001, J. Rheum. 28:2369). It is also noted that ApoA-I has been shown to bind to scavenger receptor, class B, type I (SR-BI) (Williams et al., 2000, J. Biol. Chem. 275:18897-18904). SR-BI has been indicated to play a role in the selective uptake of high density lipoprotein-cholesteryl esters (HDL-CE).

The role of innate immunity in inflammatory conditions associated with infectious disease is of great importance for clearing microbial infections and providing a control and bridge to the adaptive immune response. In addition, it is known that microbial infection, which is most typically associated with the proinflammatory Th1-type responses, can sometimes worsen the Th2-type response that is characteristic of allergic inflammation. The present inventors' data, including the finding that HIN-2 is expressed in the lung, can be upregulated during proinflammatory conditions such as occur in cystic fibrosis, binds to the scavenger receptor MARCO, and binds to bacteria, LPS and apolipoprotein A-I (notably at least two of which are also bound by MARCO), provides evidence that HIN-2 plays a role in innate immunity and inflammation. Taken together, the present inventors' data regarding the receptors and other ligands for HIN-2 provide evidence that HIN-2 has a role in inflammatory processes and microbial infection (e.g., bacterial infection, yeast infection, etc.), leading the present inventors to propose the use of HIN-2 agonists and antagonists in methods to regulate these processes. Therefore, modulation of HIN-2 expression and biological activity, including its interaction with MARCO and other ligands will be useful for targeting and regulating the innate immune response and various types of inflammation, including inflammation due to bacterial infection, inflammation due to yeast infection, apoptosis, non-infectious inflammation (e.g., including autoimmune disease), and allergic inflammation. Additionally, the data described herein suggest that HIN-2 may also have some role as a cancer marker or at a minimum, can be used to target lung tumor cells.

Accordingly, one embodiment of the present invention relates to an isolated protein comprising a HIN-2 polypeptide or homologue thereof. The HIN-2 protein has an amino acid sequence selected from: (a) an amino acid sequence selected from SEQ ID NO:2, amino acids 22-93 of SEQ ID NO:2, SEQ ID NO:4, or amino acids 22-91 of SEQ ID NO:4; (b) an amino acid sequence that is at least about 50% identical to SEQ ID NO:2, amino acids 22-93 of SEQ ID NO:2, SEQ ID NO:4, or amino acids 22-91 of SEQ ID NO:4, wherein the protein has HIN-2 biological activity; and, (c) an amino acid sequence consisting of a fragment of SEQ ID NO:2, amino acids 22-93 of SEQ ID NO:2, SEQ ID NO:4, or amino acids 22-91 of SEQ ID NO:4, and in one embodiment, the fragment has HIN-2 biological activity.

Various embodiments of the present invention are described below initially with regard to an isolated HIN-2 protein of the present invention. It is to be understood, however, that the general definitions of terms and methods and the concept of providing homologues are intended to apply to the discussion of an isolated HIN-2 receptor, also discussed below, unless otherwise modified within the specific discussion of the HIN-2 receptor.

An isolated protein, such as a HIN-2 protein, according to the present invention, is a protein (including a polypeptide or peptide) that has been removed from its natural milieu (i.e., that has been subject to human manipulation) and can include purified proteins, partially purified proteins, recombinantly produced proteins, and synthetically produced proteins, for example. As such, “isolated” does not reflect the extent to which the protein has been purified. Preferably, an isolated HIN-2 protein of the present invention is produced recombinantly. In addition, and by way of example, a “human HIN-2 protein” refers to a HIN-2 protein (generally including a homologue of a naturally occurring HIN-2 protein) from a human (Homo sapiens) or to a HIN-2 protein that has been otherwise produced from the knowledge of the structure (e.g., sequence) and perhaps the function of a naturally occurring HIN-2 protein from Homo sapiens. In other words, a human HIN-2 protein includes any HIN-2 protein that has substantially similar structure and function of a naturally occurring HIN-2 protein from Homo sapiens or that is a biologically active (i.e., has biological activity) homologue of a naturally occurring HIN-2 protein from Homo sapiens as described in detail herein. As such, a human HIN-2 protein can include purified, partially purified, recombinant, mutated/modified and synthetic proteins. According to the present invention, the terms “modification” and “mutation” can be used interchangeably, particularly with regard to the modifications/mutations to the amino acid sequences of HIN-2 or HIN-2 receptors (or nucleic acid sequences) described herein. An isolated protein useful as an antagonist or agonist according to the present invention can be isolated from its natural source, produced recombinantly or produced synthetically.

As used herein, the term “homologue” is used to refer to a protein or peptide which differs from a naturally occurring protein or peptide (i.e., the “prototype” or “wild-type” protein) by minor modifications to the naturally occurring protein or peptide, but which maintains the basic protein and side chain structure of the naturally occurring form. Such changes include, but are not limited to: changes in one or a few amino acid side chains; changes one or a few amino acids, including deletions (e.g., a truncated version of the protein or peptide) insertions and/or substitutions; changes in stereochemistry of one or a few atoms; and/or minor derivatizations, including but not limited to: methylation, glycosylation, phosphorylation, acetylation, myristoylation, prenylation, palmitation, amidation and/or addition of glycosylphosphatidyl inositol. A homologue can have either enhanced, decreased, or substantially similar properties as compared to the naturally occurring protein or peptide. A homologue can include an agonist of a protein or an antagonist of a protein.

Homologues can be the result of natural allelic variation or natural mutation. A naturally occurring allelic variant of a nucleic acid encoding a protein is a gene that occurs at essentially the same locus (or loci) in the genome as the gene which encodes such protein, but which, due to natural variations caused by, for example, mutation or recombination, has a similar but not identical sequence. Allelic variants typically encode proteins having similar activity to that of the protein encoded by the gene to which they are being compared. One class of allelic variants can encode the same protein but have different nucleic acid sequences due to the degeneracy of the genetic code. Allelic variants can also comprise alterations in the 5′ or 3′ untranslated regions of the gene (e.g., in regulatory control regions). Allelic, variants are well known to those skilled in the art.

Homologues can be produced using techniques known in the art for the production of proteins including, but not limited to, direct modifications to the isolated, naturally occurring protein, direct protein synthesis, or modifications to the nucleic acid sequence encoding the protein using, for example, classic or recombinant DNA techniques to effect random or targeted mutagenesis.

Modifications in HIN-2 homologues (or HIN-2 receptor homologues), as compared to the wild-type protein, either agonize, antagonize, or do not substantially change, the basic biological activity of the HIN-2 homologue as compared to the naturally occurring protein, HIN-2. In general, the biological activity or biological action of a protein refers to any function(s) exhibited or performed by the protein that is ascribed to the naturally occurring form of the protein as measured or observed in vivo (i.e., in the natural physiological environment of the protein) or in vitro (i.e., under laboratory conditions). Modifications of a protein, such as in a homologue or mimetic (discussed below), may result in proteins having the same biological activity as the naturally occurring protein, or in proteins having decreased or increased biological activity as compared to the naturally occurring protein. Modifications which result in a decrease in protein expression or a decrease in the activity of the protein, can be referred to as inactivation (complete or partial), down-regulation, or decreased action of a protein. Similarly, modifications which result in an increase in protein expression or an increase in the activity of the protein, can be referred to as amplification, overproduction, activation, enhancement, up-regulation or increased action of a protein.

According to the present invention, an isolated HIN-2 protein, including a biologically active homologue or fragment thereof, has at least one characteristic of biological activity of activity a wild-type, or naturally occurring HIN-2 protein (which can vary depending on whether the homologue or fragment is an agonist, antagonist, or mimic of HIN-2). The biological activity of HIN-2 can include, but is not limited to, binding to at least one HIN-2 receptor; binding to at least one natural binding ligand of HIN-2 (e.g., LPS, bacteria, yeast, ApoA-I); regulation of the ability of a cell expressing a HIN-2 receptor (e.g., MARCO) to bind to and/or phagocytose bacteria; regulation of the ability of a cell expressing a HIN-2 receptor to bind to inflammatory mediators; regulation of the production of inflammatory mediators by a cell expressing the HIN-2 receptor or by other cells at the site of inflammation; regulation of a response to yeast infection; regulation of inflammation and/or the innate immune response at a site where the HIN-2 is expressed or administered; and a marker of inflammation or an innate immune response. Methods of detecting and measuring HIN-2 biological activity (and measuring HIN-2 agonist or antagonist activity) include, but are not limited to, measurement of transcription of HIN-2, measurement of translation of HIN-2, measurement of secretion of HIN-2, measurement of binding of HIN-2 to a HIN-2 receptor, measurement of binding of HIN-2 to a HIN-2 binding ligand (e.g., a bacterium, yeast, LPS, ApoA-I), measurement of an increase or decrease in any of the above-described characteristics of HIN-2 or its receptor MARCO in innate immunity and inflammation (e.g., phagocytosis, microbial clearance, production of inflammatory mediators, identification of type of inflammatory mediators produced at a site). It is noted that an isolated HIN-2 protein of the present invention (including homologues) is not required to have HIN-2 activity. A HIN-2 protein can be a truncated, mutated or inactive protein, for example. Such proteins are useful in diagnostic assays or some screening assays, for example, or for other purposes such as antibody production.

Methods to measure protein expression levels of selected genes of this invention, include, but are not limited to: western blotting, immunocytochemistry, flow cytometry or other immunologic-based assays; assays based on a property of the protein including but not limited to DNA binding, ligand binding, or interaction with other protein partners. Binding assays are also well known in the art. For example, a BIAcore machine can be used to determine the binding constant of a complex between two proteins. The dissociation constant for the complex can be determined by monitoring changes in the refractive index with respect to time as buffer is passed over the chip (O'Shannessy et al. Anal. Biochem. 212:457-468 (1993); Schuster et al., Nature 365:343-347 (1993)). Other suitable assays for measuring the binding of one protein to another include, for example, immunoassays such as enzyme linked immunoabsorbent assays (ELISA) and radioimmunoassays (RIA), or determination of binding by monitoring the change in the spectroscopic or optical properties of the proteins through fluorescence, UV absorption, circular dichrosim, or nuclear magnetic resonance (NMR).

To measure inflammation, standard assays include, but are not limited to, measurement of cytokine production by cells at a site of inflammation or a site of interest, measurement of upregulation or downregulation of cellular markers of activation, measurement of cell type infiltrating a site, measurement of immediate type hypersensitivity reactions, measurement of delayed type hypersensitivity reactions, measurement of airway hyperresponsiveness (for allergic inflammation, see, e.g., U.S. Pat. No. 6,248,723, incorporated by reference in its entirety). All of these parameters can be measured using techniques standard in the art, including biological assays, in situ assays, flow cytometry, immunoassays, in vivo assays in animal models, etc.

As used herein, the phrase “HIN-2 agonist” refers to any compound that is characterized by the ability to agonize (e.g., stimulate, induce, increase, enhance, or mimic) the biological activity of a naturally occurring HIN-2 as described herein (e.g., by interaction/binding with and/or activation of a HIN-2 receptor). Similarly, the phrase “HIN-2 receptor agonist compound” or “HIN-2 receptor agonist” (or “MARCO agonist”) refers to any compound that is characterized by its ability to agonize (e.g., stimulate, induce, increase, enhance) the biological activity of a naturally occurring HIN-2 receptor as described herein (e.g., by interaction/binding with ligands of the HIN-2 receptor and mimicking or enhancing the biological activity of the HIN-2 receptor). More particularly, a HIN-2 agonist can include, but is not limited to, a protein, peptide, or nucleic acid that selectively binds to and activates or increases the activation of a HIN-2 receptor or other is HIN-2 binding ligand, or otherwise mimics or enhances the activity of the natural ligand, HIN-2, and includes any HIN-2 homologue, binding protein (e.g., an antibody), agent that interacts with HIN-2, or any suitable product of drug/compound/peptide design or selection which is characterized by its ability to agonize (e.g., stimulate, induce, increase, enhance) the biological activity of a naturally occurring HIN-2 protein in a manner similar to the natural agonist, HIN-2 (e.g., by interaction/binding with and/or direct or indirect activation of a HIN-2 receptor). Agonists of HIN-2 of the present invention can be useful in methods for regulating the innate immune response and inflammation, and particularly, in methods for regulating binding and phagocytosis of bacteria, regulating yeast infection, regulating inflammatory conditions, and may also be useful for targeting antigen to immune cells and/or regulating the presentation of antigen to immune accessory cells. The use of HIN-2 agonists typically produce at least one result, as compared to in the absence of the agonist which includes, but is not limited to: (1) binding to a HIN-2 receptor; (2) regulation of the ability of a cell expressing a HIN-2 receptor to bind to and/or phagocytose bacteria or yeast; (3) regulation of the ability of a cell expressing a HIN-2 receptor to bind to inflammatory mediators, such as LPS; (4) regulation of the production of inflammatory mediators by a cell expressing the HIN-2 receptor or by other cells at the site of inflammation; (5) regulation of inflammation and/or the innate immune response at a site where the agonist is expressed or administered. A HIN-2 receptor agonist can include, but is not limited to, a protein, peptide, nucleic acid, or any suitable product of drug/compound/peptide design or selection that mimics or enhances the activity of the natural HIN-2 receptor. Agonists of HIN-2 receptors of the present invention can also be useful in methods for regulating the innate immune response and inflammation, and particularly, in methods for regulating binding and phagocytosis of bacteria or yeast, regulating inflammatory conditions, and may also be useful for targeting antigen to immune cells and/or regulating the presentation of antigen to immune accessory cells.

The phrase, “HIN-2 antagonist” or “HIN-2 receptor antagonist” refers to any compound which inhibits (e.g., antagonizes, reduces, decreases, blocks, reverses, or alters) the effect of a HIN-2 agonist or a HIN-2 receptor agonist, respectively, as described above. More particularly, a HIN-2 antagonist is capable of associating with a HIN-2 receptor or other HIN-2 binding partner, or otherwise acts in a manner relative to HIN-2 activity, such that the biological activity of the receptor or binding partner, or of the natural agonist HIN-2, is decreased in a manner that is antagonistic (e.g., against, a reversal of, contrary to) to the natural action of HIN-2 or the HIN-2 receptor upon binding to HIN-2 (such as by competitive inhibition of the interaction between HIN-2 and its receptor, by blocking the HIN-2 receptor from HIN-2 binding, or by inducing a different effect on the receptor as compared to the effect induced by HIN-2). Similarly, a HIN-2 receptor antagonist is capable of mimicking the structure of a natural HIN-2 receptor, and/or associating with HIN-2 or other binding ligands of the HIN-2 receptor in a manner that is antagonistic (e.g., against, a reversal of, contrary to) to the natural action of the HIN-2 receptor or the HIN-2 receptor upon binding to HIN-2 or another HIN-2 receptor ligand. Such antagonists can include, but are not limited to, steroidal or non-steroidal compounds; a protein, peptide, or nucleic acid (including ribozymes and antisense) or product of drug/compound/peptide design or selection that provides the antagonistic effect. Contact of a HIN-2 receptor, or a HIN-2 ligand, with a HIN-2 antagonist typically produces at least one result, as compared to in the absence of the antagonist, which includes, but is not limited to: (1) inhibited or decreased binding of the natural HIN-2 receptor ligand (e.g., HIN-2 or other ligands) to the HIN-2 receptor; (2) regulation of the ability of a cell expressing a HIN-2 receptor to bind to and/or phagocytose bacteria or yeast; (3) regulation of the ability of a cell expressing a HIN-2 receptor to bind to inflammatory mediators, such as LPS; (4) regulation of the production of inflammatory mediators by a cell expressing the HIN-2 receptor or by other cells at the site of inflammation; (5) regulation of inflammation and/or the innate immune response at a site where the agonist is expressed or administered.

According to the present invention, a ribozyme typically contains stretches of complementary RNA bases that can base-pair with a target RNA ligand, including the RNA molecule itself, giving rise to an active site of defined structure that can cleave the bound RNA molecule (See Maulik et al., 1997, supra). Therefore, a ribozyme can serve as a targeting delivery vehicle for a nucleic acid molecule, or alternatively, the ribozyme can target and bind to RNA encoding HIN-2 or a HIN-2 receptor, for example, and thereby effectively inhibit the translation of HIN-2 or the HIN-2 receptor.

As used herein, an anti-sense nucleic acid molecule is defined as an isolated nucleic acid molecule that reduces expression of a HIN-2 protein or a HIN-2 receptor by hybridizing under high stringency conditions to a gene encoding the HIN-2 protein or receptor, respectively. Such a nucleic acid molecule is sufficiently similar to the nucleic acid sequence encoding the HIN-2 protein or receptor that the molecule is capable of hybridizing under high stringency conditions to the coding strand of the gene or RNA encoding the natural HIN-2 protein or receptor. In a particularly preferred embodiment, an anti-sense nucleic acid molecule of the present invention is the exact complement of the coding region of a HIN-2 protein or receptor. It is noted that the anti-sense of the coding region does not necessarily include the anti-sense of the stop codon.

Homologues of HIN-2, including peptide and non-peptide agonists and antagonists of HIN-2, can be products of drug design or selection and can be produced using various methods known in the art. Such homologues can be referred to as mimetics. A mimetic refers to any peptide or non-peptide compound that is able to mimic the biological action of a naturally occurring peptide, often because the mimetic has a basic structure that mimics the basic structure of the naturally occurring peptide and/or has the salient biological properties of the naturally occurring peptide. Mimetics can include, but are not limited to: peptides that have substantial modifications from the prototype such as no side chain similarity with the naturally occurring peptide (such modifications, for example, may decrease its susceptibility to degradation); anti-idiotypic and/or catalytic antibodies, or fragments thereof; non-proteinaceous portions of an isolated protein (e.g., carbohydrate structures); or synthetic or natural organic molecules, including nucleic acids and drugs identified through combinatorial chemistry, for example. Such mimetics can be designed, selected and/or otherwise identified using a variety of methods known in the art. Various methods of drug design, useful to design or select mimetics or other therapeutic compounds useful in the present invention are disclosed in Maulik et al., 1997, Molecular Biotechnology: Therapeutic Applications and Strategies, Wiley-Liss, Inc., which is incorporated herein by reference in its entirety.

A mimetic can be obtained, for example, from molecular diversity strategies (a combination of related strategies allowing the rapid construction of large, chemically diverse molecule libraries), libraries of natural or synthetic compounds, in particular from chemical or combinatorial libraries (i.e., libraries of compounds that differ in sequence or size but that have the similar building blocks) or by rational, directed or random drug design. See for example, Maulik et al., supra.

In a molecular diversity strategy, large compound libraries are synthesized, for example, from peptides, oligonucleotides, carbohydrates and/or synthetic organic molecules, using biological, enzymatic and/or chemical approaches. The critical parameters in developing a molecular diversity strategy include subunit diversity, molecular size, and library diversity. The general goal of screening such libraries is to utilize sequential application of combinatorial selection to obtain high-affinity ligands for a desired target, and then to optimize the lead molecules by either random or directed design strategies. Methods of molecular diversity are described in detail in Maulik, et al., ibid.

Maulik et al. also disclose, for example, methods of directed design, in which the user directs the process of creating novel molecules from a fragment library of appropriately selected fragments; random design, in which the user uses a genetic or other algorithm to randomly mutate fragments and their combinations while simultaneously applying a selection criterion to evaluate the fitness of candidate ligands; and a grid-based approach in which the user calculates the interaction energy between three dimensional receptor structures and small fragment probes, followed by linking together of favorable probe sites.

In one embodiment of the present invention, a HIN-2 protein has an amino acid sequence that comprises, consists essentially of, or consists of, SEQ ID NO:2, amino acids 22-93 of SEQ ID NO:2, SEQ ID NO:4, or amino acids 22-91 of SEQ ID NO:4. SEQ ID NO:2 represents a human HIN-2 preprotein (encoded by nucleic acid sequence SEQ ID NO:1); amino acids 22-93 of SEQ ID NO:2 represent the mature human HIN-2 protein; SEQ ID NO:4 represents a mouse HIN-2 preprotein of the present invention (encoded by nucleic acid sequence SEQ ID NO:3); and amino acids 22-91 of SEQ ID NO:4 represent the mature mouse HIN-2 protein. The present invention also includes homologues of SEQ ID NO:2, amino acids 22-93 of SEQ ID NO:2, SEQ ID NO:4, or amino acids 22-91 of SEQ ID NO:4, or fragments of SEQ ID NO:2, amino acids 22-93 of SEQ ID NO:2, SEQ ID NO:4, or amino acids 22-91 of SEQ ID NO:4, wherein the homologue or fragment has HIN-2 biological activity (including agonist or antagonist activity), as described previously herein.

In one embodiment, a HIN-2 protein of the present invention, including a HIN-2 homologue, has an amino acid sequence that is at least about 50% identical to an amino acid sequence of selected from SEQ ID NO:2, amino acids 22-93 of SEQ ID NO:2, SEQ ID NO:4, or amino acids 22-91 of SEQ ID NO:4, over the full length of any of such sequences, wherein the protein has HIN-2 biological activity (which can include agonist or antagonist activity). In another embodiment, a HIN-2 protein of the present invention has an amino acid sequence that is at least about 55% identical, or at least about 60% identical, or at least about 65% identical, or at least about 70% identical, or at least about 75% identical, or at least about 80% identical, or at least about 85% identical, or at least about 90% identical, or at least about 95% identical, or at least about 96% identical, or at least about 97% identical, or at least about 98% identical, or at least about 99% identical to SEQ ID NO:2, amino acids 22-93 of SEQ ID NO:2, SEQ IDNO:4, or amino acids 22-91 of SEQ ID NO:4, over the full length of any of such sequences.

In another embodiment, a HIN-2 protein of the present invention, including a HIN-2 homologue, has an amino acid sequence that is at least about 50% identical to SEQ ID NO:2, amino acids 22-93 of SEQ ID NO:2, SEQ ID NO:4, or amino acids 22-91 of SEQ ID NO:4, over at least 25 amino acids of any of such sequences. In one embodiment, the HIN-2 protein of the present invention has an amino acid sequence that is at least about 55% identical, or at least about 60% identical, or at least about 65% identical, or at least about 70% identical, or at least about 75% identical, or at least about 80% identical, or at least about 85% identical, or at least about 90% identical, or at least about 95% identical, or at least about 96% identical, or at least about 97% identical, or at least about 98% identical, or at least about 99% identical to SEQ ID NO:2, amino acids 22-93 of SEQ ID NO:2, SEQ ID NO:4, or amino acids 22-91 of SEQ ID NO:4, over at least 35 amino acids, or over at least 40 amino acids, or over at least 45 amino acids, or over at least 50 amino acids, or over at least 55 amino acids, or over at least 60 amino acids, or over at least 65 amino acids, or over at least 70 amino acids, or over at least 75 amino acids, or over at least 80 amino acids, or over at least 85 amino acids, or over at least 90 amino acids, of any of the above-identified sequences. In a preferred embodiment, such a protein has at least one biological activity of HIN-2, and can be an agonist or an antagonist of HIN-2.

In one embodiment ofthe present invention, a HIN-2 homologue according to the present invention has an amino acid sequence that is less than about 100% identical to SEQ ID NO:2, amino acids 22-93 of SEQ ID NO:2, SEQ ID NO:4, or amino acids 22-91 of SEQ ID NO:4. In another aspect of the invention, a HIN-2 homologue according to the present invention has an amino acid sequence that is less than about 99% identical to any of the above-identified amino acid sequences, and in another embodiment, is less than is less than 98% identical to any of the above-identified amino acid sequences, and in another embodiment, is less than 97% identical to any of the above-identified amino acid sequences, and in another embodiment, is less than 96% identical to any of the above-identified amino acid sequences, and in another embodiment, is less than 95% identical to any of the above-identified amino acid sequences, and in another embodiment, is less than 94% identical to any of the above-identified amino acid sequences, and in another embodiment, is less than 93% identical to any of the above-identified amino acid sequences, and in another embodiment, is less than 92% identical to any of the above-identified amino acid sequences, and in another embodiment, is less than 91% identical to any of the above-identified amino acid sequences, and in another embodiment, is less than 90% identical to any of the above-identified amino acid sequences, and so on, in increments of whole integers.

As used herein, unless otherwise specified, reference to a percent (%) identity refers to an evaluation of homology which is performed using: (1) a BLAST 2.0 Basic BLAST homology search using blastp for amino acid searches, blastn for nucleic acid searches, and blastX for nucleic acid searches and searches of translated amino acids in all 6 open reading frames, all with standard default parameters, wherein the query sequence is filtered for low complexity regions by default (described in Altschul, S. F., Madden, T. L., Schääffer, A. A., Zhang, J., Zhang, Z., Miller, W. & Lipman, D. J. (1997) “Gapped BLAST and PSI-BLAST: a new generation of protein database search programs.” Nucleic Acids Res. 25:3389-3402, incorporated herein by reference in its entirety); (2) a BLAST 2 alignment (using the parameters described below); (3) and/or PSI-BLAST with the standard default parameters (Position-Specific Iterated BLAST). It is noted that due to some differences in the standard parameters between BLAST 2.0 Basic BLAST and BLAST 2, two specific sequences might be recognized as having significant homology using the BLAST 2 program, whereas a search performed in BLAST 2.0 Basic BLAST using one of the sequences as the query sequence may not identify the second sequence in the top matches. In addition, PSI-BLAST provides an automated, easy-to-use version of a “profile” search, which is a sensitive way to look for sequence homologues. The program first performs a gapped BLAST database search. The PSI-BLAST program uses the information from any significant alignments returned to construct a position-specific score matrix, which replaces the query sequence for the next round of database searching. Therefore, it is to be understood that percent identity can be determined by using any one of these programs.

Two specific sequences can be aligned to one another using BLAST 2 sequence as described in Tatusova and Madden, (1999), “Blast 2 sequences—a new tool for comparing protein and nucleotide sequences”, FEMS Microbiol Lett. 174:247-250, incorporated herein by reference in its entirety. BLAST 2 sequence alignment is performed in blastp or blastn using the BLAST 2.0 algorithm to perform a Gapped BLAST search (BLAST 2.0) between the two sequences allowing for the introduction of gaps (deletions and insertions) in the resulting alignment. For purposes of clarity herein, a BLAST 2 sequence alignment is performed using the standard default parameters as follows.

For blastn, using 0 BLOSUM62 matrix:
Reward for match=1
Penalty for mismatch=−2
Open gap (5) and extension gap (2) penalties
gap x_dropoff (50) expect (10) word size (11) filter (on)

For blastp, using 0 BLOSUM62 matrix:
Open gap (11) and extension gap (1) penalties
gap x_dropoff(50) expect (10) word size (3) filter (on).

A HIN-2 protein of the present invention, including a HIN-2 homologue, can also include proteins having an amino acid sequence comprising at least 25 contiguous amino acid residues of SEQ ID NO:2, amino acids 22-93 of SEQ ID NO:2, SEQ ID NO:4, or amino acids 22-91 of SEQ ID NO:4 (i.e., 25 contiguous amino acid residues having 100% identity with 25 contiguous amino acids of any of the above-identified sequences). In one embodiment, a HIN-2 homologue of the present invention includes proteins having amino acid sequences comprising at least about 30, or at least about 40, or at least about 45, or at least about 50, or at least about 55, or at least about 60, or at least about 65, or at least about 70, or at least about 75, or at least about 80, or at least about 85, or at least about 90, contiguous amino acid residues of SEQ ID NO:2, amino acids 22-93 of SEQ ID NO:2, SEQ ID NO:4, or amino acids 22-91 of SEQ ID NO:4. In one embodiment, such a protein has HIN-2 biological activity (including agonist or antagonist activity).

According to the present invention, the term “contiguous” or “consecutive”, with regard to nucleic acid or amino acid sequences described herein, means to be connected in an unbroken sequence. For example, for a first sequence to comprise 30 contiguous (or consecutive) amino acids of a second sequence, means that the first sequence includes an unbroken sequence of 30 amino acid residues that is 100% identical to an unbroken sequence of 30 amino acid residues in the second sequence. Similarly, for a first sequence to have “100% identity” with a second sequence means that the first sequence exactly matches the second sequence with no gaps between nucleotides or amino acids.

In another embodiment, a HIN-2 homologue includes a protein having an amino acid sequence that is sufficiently similar to a naturally occurring HIN-2 amino acid sequence that a nucleic acid sequence encoding the homologue is capable of hybridizing under moderate, high, or very high stringency conditions (described below) to (i.e., with) a nucleic acid molecule encoding the naturally occurring HIN-2 protein (i.e., to the complement of the nucleic acid strand encoding the naturally occurring HIN-2 amino acid sequence). Preferably, a HIN-2 protein, including a HIN-2 homologue, is encoded by a nucleic acid sequence that hybridizes under moderate, high or very high stringency conditions to the complement of a nucleic acid sequence that encodes a protein comprising an amino acid sequence represented by SEQ ID NO:2, amino acids 22-93 of SEQ ID NO:2, SEQ ID NO:4, or amino acids 22-91 of SEQ ID NO:4. Even more preferably, a HIN-2 protein of the present invention, including a HIN-2 homologue, is encoded by a nucleic acid sequence that hybridizes under moderate, high or very high stringency conditions to the complement of the coding region of a nucleic acid sequence selected from SEQ ID NO:1, to nucleotides 158-373 of SEQ ID NO:1, SEQ ID NO:3, or nucleotides 147-356 of SEQ ID NO:3. Such hybridization conditions are described in detail below. A nucleic acid sequence complement of nucleic acid sequence encoding a HIN-2 protein of the present invention refers to the nucleic acid sequence of the nucleic acid strand that is complementary to the strand which encodes the HIN-2 protein. It will be appreciated that a double stranded DNA which encodes a given amino acid sequence comprises a single strand DNA and its complementary strand having a sequence that is a complement to the single strand DNA. As such, nucleic acid molecules of the present invention can be either double-stranded or single-stranded, and include those nucleic acid molecules that form stable hybrids under stringent hybridization conditions with a nucleic acid sequence that encodes an amino acid sequence of HIN-2, and/or with the complement of the nucleic acid sequence that encodes any of such amino acid sequences. Methods to deduce a complementary sequence are known to those skilled in the art. It should be noted that since amino acid sequencing and nucleic acid sequencing technologies are not entirely error-free, the sequences presented herein, at best, represent apparent sequences of HIN-2 proteins of the present invention.

As used herein, reference to hybridization conditions refers to standard hybridization conditions under which nucleic acid molecules are used to identify similar nucleic acid molecules. Such standard conditions are disclosed, for example, in Sambrook et al., Molecular Cloning: A Laboratory Manual, Cold Spring Harbor Labs Press, 1989. Sambrook et al., ibid., is incorporated by reference herein in its entirety (see specifically, pages 9.31-9.62). In addition, formulae to calculate the appropriate hybridization and wash conditions to achieve hybridization permitting varying degrees of mismatch of nucleotides are disclosed, for example, in Meinkoth et al., 1984, Anal. Biochem. 138, 267-284; Meinkoth et al., ibid., is incorporated by reference herein in its entirety.

More particularly, moderate stringency hybridization and washing conditions, as referred to herein, refer to conditions which permit isolation of nucleic acid molecules having at least about 70% nucleic acid sequence identity with the nucleic acid molecule being used to probe in the hybridization reaction (i.e., conditions permitting about 30% or less mismatch of nucleotides). High stringency hybridization and washing conditions, as referred to herein, refer to conditions which permit isolation of nucleic acid molecules having at least about 80% nucleic acid sequence identity with the nucleic acid molecule being used to probe in the hybridization reaction (i.e., conditions permitting about 20% or less mismatch of nucleotides). Very high stringency hybridization and washing conditions, as referred to herein, refer to conditions which permit isolation of nucleic acid molecules having at least about 90% nucleic acid sequence identity with the nucleic acid molecule being used to probe in the hybridization reaction (i.e., conditions permitting about 10% or less mismatch of nucleotides). As discussed above, one of skill in the art can use the formulae in Meinkoth et al., ibid. to calculate the appropriate hybridization and wash conditions to achieve these particular levels of nucleotide mismatch. Such conditions will vary, depending on whether DNA:RNA or DNA:DNA hybrids are being formed. Calculated melting temperatures for DNA:DNA hybrids are 10° C. less than for DNA:RNA hybrids. In particular embodiments, stringent hybridization conditions for DNA:DNA hybrids include hybridization at an ionic strength of 6×SSC (0.9 M Na⁺) at a temperature of between about 20° C. and about 35° C. (lower stringency), more preferably, between about 28° C. and about 40° C. (more stringent), and even more preferably, between about 35° C. and about 45° C. (even more stringent), with appropriate wash conditions. In particular embodiments, stringent hybridization conditions for DNA:RNA hybrids include hybridization at an ionic strength of 6×SSC (0.9 M Na⁺) at a temperature of between about 30° C. and about 45° C., more preferably, between about 38° C. and about 50° C., and even more preferably, between about 45° C. and about 55° C., with similarly stringent wash conditions. These values are based on calculations of a melting temperature for molecules larger than about 100 nucleotides, 0% formamide and a G+C content of about 40%. Alternatively, Tₘ can be calculated empirically as set forth in Sambrook et al., supra, pages 9.31 to 9.62. In general, the wash conditions should be as stringent as possible, and should be appropriate for the chosen hybridization conditions. For example, hybridization conditions can include a combination of salt and temperature conditions that are approximately 20-25° C. below the calculated Tₘ of a particular hybrid, and wash conditions typically include a combination of salt and temperature conditions that are approximately 12-20° C. below the calculated Tₘ of the particular hybrid. One example of hybridization conditions suitable for use with DNA:DNA hybrids includes a 2-24 hour hybridization in 6×SSC (50% formamide) at about 42° C., followed by washing steps that include one or more washes at room temperature in about 2×SSC, followed by additional washes at higher temperatures and lower ionic strength (e.g., at least one wash as about 37° C. in about 0.1×-0.5×SSC, followed by at least one wash at about 68° C. in about 0.1×-0.5×SSC).

HIN-2 proteins of the present invention also include expression products of gene fusions (for example, used to overexpress soluble, active forms of the recombinant protein), of mutagenized genes (such as genes having codon modifications to enhance gene transcription and translation), and of truncated genes (such as genes having signal sequences removed which are poorly tolerated in a particular recombinant host).

The minimum size of a HIN-2 protein and/or homologue of the present invention is, in one aspect, a size sufficient to have HIN-2 biological activity, including agonist or antagonist activity. In another embodiment, a protein of the present invention is at least 13 amino acids in length, or at least about 15 amino acids in length, or at least about 20 amino acids in length, or at least about 25 amino acids in length, or at least about 30 amino acids in length, or at least about 35 amino acids in length, or at least about 40 amino acids in length, or at least about 45 amino acids in length, or at least about 50 amino acids in length, or at least about 55 amino acids in length, or at least about 60 amino acids in length, or at least about 65 amino acids in length, or at least about 70 amino acids in length, or at least about 75 amino acids in length, or at least about 80 amino acids in length, or at least about 85 amino acids in length, or at least about 90 amino acids in length. There is no limit, other than a practical limit, on the maximum size of such a protein in that the protein can include a portion of a HIN-2 protein or a full-length HIN-2 protein, plus additional sequence (e.g., a fusion protein sequence), if desired.

The present invention also includes a fusion protein that includes a HIN-2-containing domain (i.e., an amino acid sequence for a HIN-2 protein according to the present invention) attached to one or more fusion segments. Suitable fusion segments for use with the present invention include, but are not limited to, segments that can: enhance a protein's stability; provide other desirable biological activity (e.g., a therapeutic protein/peptide to be delivered to a site); and/or assist with the purification of a HIN-2 protein (e.g., by affinity chromatography). A suitable fusion segment can be a domain of any size that has the desired function (e.g., imparts increased stability, solubility, biological activity; and/or simplifies purification of a protein). Fusion segments can be joined to amino and/or carboxyl termini of the HIN-2-containing domain of the protein and can be susceptible to cleavage in order to enable straight-forward recovery of HIN-2. Fusion proteins are preferably produced by culturing a recombinant cell transfected with a fusion nucleic acid molecule that encodes a protein including the fusion segment attached to either the carboxyl and/or amino terminal end of a HIN-2-containing domain.

In one embodiment of the present invention, any of the amino acid sequences described herein can be produced with from at least one, and up to about 20, additional heterologous amino acids flanking each of the C— and/or N-terminal ends of the specified amino acid sequence. The resulting protein or polypeptide can be referred to as “consisting essentially of” the specified amino acid sequence. According to the present invention, the heterologous amino acids are a sequence of amino acids that are not naturally found (i.e., not found in nature, in vivo) flanking the specified amino acid sequence, or that are not related to the function of the specified amino acid sequence, or that would not be encoded by the nucleotides that flank the naturally occurring nucleic acid sequence encoding the specified amino acid sequence as it occurs in the gene, if such nucleotides in the naturally occurring sequence were translated using standard codon usage for the organism from which the given amino acid sequence is derived. Similarly, the phrase “consisting essentially of”, when used with reference to a nucleic acid sequence herein, refers to a nucleic acid sequence encoding a specified amino acid sequence that can be flanked by from at least one, and up to as many as about 60, additional heterologous nucleotides at each of the 5′ and/or the 3′ end of the nucleic acid sequence encoding the specified amino acid sequence. The heterologous nucleotides are not naturally found (i.e., not found in nature, in vivo) flanking the nucleic acid sequence encoding the specified amino acid sequence as it occurs in the natural gene or do not encode a protein that imparts any additional function to the protein or changes the function of the protein having the specified amino acid sequence.

Another embodiment of the present invention relates to a composition comprising at least about 500 ng, and preferably at least about 1 μg, and more preferably at least about 5 μg, and more preferably at least about 10 μg, and more preferably at least about 25 μg, and more preferably at least about 50 μg, and more preferably at least about 75 μg, and more preferably at least about 100 μg, and more preferably at least about 250 μg, and more preferably at least about 500 μg, and more preferably at least about 750 μg, and more preferably at least about 1 mg, and more preferably at least about 5 mg, of an isolated HIN-2 protein comprising any of the HIN-2 proteins or homologues thereof discussed herein. Such a composition of the present invention can include any carrier with which the protein is associated by virtue of the protein preparation method, a protein purification method, or a preparation of the protein for use in an in vitro, ex vivo, or in vivo method according to the present invention. For example, such a carrier can include any suitable excipient, buffer and/or delivery vehicle, such as a pharmaceutically acceptable carrier (discussed below), which is suitable for combining with the HIN-2 protein of the present invention so that the protein can be used in vitro, ex vivo or in vivo according to the present invention.

Further embodiments of the present invention include nucleic acid molecules that encode any of the above-identified HIN-2 proteins, including a homologue or fragment thereof. In a preferred embodiment, nucleic acid molecules encoding HIN-2 include the nucleic acid sequence represented by nucleotides 95-373 of SEQ ID NO:1, by nucleotides 158-373 of SEQ ID NO:1, by nucleotides 84-356 of SEQ ID NO:3, by nucleotides 147-356 of SEQ ID NO:3, or homologues thereof as described herein. In accordance with the present invention, an isolated polynucleotide, or an isolated nucleic acid molecule, is a nucleic acid molecule that has been removed from its natural milieu (i.e., that has been subject to human manipulation), its natural milieu being the genome or chromosome in which the nucleic acid molecule is found in nature. As such, “isolated” does not necessarily reflect the extent to which the nucleic acid molecule has been purified, but indicates that the molecule does not include an entire genome or an entire chromosome in which the nucleic acid molecule is found in nature. An isolated nucleic acid molecule can include a gene or a portion of a gene (e.g., the regulatory region or promoter). An isolated nucleic acid molecule that includes a gene is not a fragment of a chromosome that includes such gene, but rather includes the coding region and regulatory regions associated with the gene, but no additional genes naturally found on the same chromosome. An isolated nucleic acid molecule can also include a specified nucleic acid sequence flanked by (i.e., at the 5′ and/or the 3′ end of the sequence) additional nucleic acids that do not normally flank the specified nucleic acid sequence in nature (i.e., heterologous sequences). Isolated nucleic acid molecule can include DNA, RNA (e.g., mRNA), or derivatives of either DNA or RNA (e.g., cDNA). Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a protein. Preferably, an isolated nucleic acid molecule of the present invention is produced using recombinant DNA technology (e.g., polymerase chain reaction (PCR) amplification, cloning) or chemical synthesis. If the polynucleotide is an oligonucleotide, such as a probe or primer, the oligonucleotide preferably ranges from about 5 to about 50 or about 500 nucleotides, more preferably from about 10 to about 40 nucleotides, and most preferably from about 15 to about 40 nucleotides in length.

In one embodiment, an oligonucleotide of the present invention, a plurality of oligonucleotides of the present invention, or other nucleic acids of the invention, are immobilized on a substrate, such as for use in a screening assay. In general, an array, an oligonucleotide, a cDNA, or genomic DNA, that is a portion of a gene (e.g., Hin2) occupies a known location on a substrate (e.g., is immobilized on a substrate). A nucleic acid target sample is hybridized with the immobilized nucleic acid and then the amount of target nucleic acids hybridized to each probe in the array/assay is quantified. One preferred quantifying method is to use confocal microscope and fluorescent labels. The Affymetrix GeneChip™ Array system (Affymetrix, Santa Clara, Calif.) and the Atlas™ Human cDNA Expression Array system are particularly suitable for quantifying the hybridization; however, it will be apparent to those of skill in the art that any similar systems or other effectively equivalent detection methods can also be used. In a particularly preferred embodiment, one can use the knowledge of the genes described herein to design novel arrays of polynucleotides, cDNAs or genomic DNAs for screening methods described herein. Such novel pluralities of polynucleotides are contemplated to be a part of the present invention.

In one embodiment, the polynucleotide probes are conjugated to detectable markers. Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads. Preferably, the polynucleotide probes are immobilized on a substrate such as: artificial membranes, organic supports, biopolymer supports and inorganic supports.

Suitable nucleic acid samples for screening on an array contain transcripts of interest or nucleic acids derived from the transcripts of interest (i.e., transcripts derived from the PR-regulated genes of the present invention). As used herein, a nucleic acid derived from a transcript refers to a nucleic acid for whose synthesis the mRNA transcript or a subsequence thereof has ultimately served as a template. Thus, a cDNA reverse transcribed from a transcript, an RNA transcribed from that cDNA, a DNA amplified from the cDNA, an RNA transcribed from the amplified DNA, etc., are all derived from the transcript and detection of such derived products is indicative of the presence and/or abundance of the original transcript in a sample. Thus, suitable samples include, but are not limited to, transcripts of the gene or genes, cDNA reverse transcribed from the transcript, cRNA transcribed from the cDNA, DNA amplified from the genes, RNA transcribed from amplified DNA, and the like.

Isolated nucleic acid molecules can include, for example, Hin-2 genes; natural allelic variants of such genes; Hin-2 coding regions, regulatory regions (e.g. promoter regions), or portions thereof; and Hin-2 coding and/or regulatory regions modified by nucleotide insertions, deletions, substitutions, and/or inversions in a manner such that the modifications do not substantially interfere with the nucleic acid molecule's ability to encode a HIN-2 protein or homologue of the present invention or to form stable hybrids under stringent conditions with natural gene isolates. An isolated Hin-2 nucleic acid molecule can include degeneracies. As used herein, nucleotide degeneracies refers to the phenomenon that one amino acid can be encoded by different nucleotide codons. Thus, the nucleic acid sequence of a nucleic acid molecule that encodes a HIN-2 protein of the present invention can vary due to degeneracies. It is noted that an isolated Hin-2 nucleic acid molecule of the present invention is not required to encode a protein having HIN-2 activity. A Hin-2 nucleic acid molecule can encode a truncated, mutated or inactive protein, for example. Such nucleic acid molecules and the proteins encoded by such nucleic acid molecules are useful in diagnostic assays, for example, or for other purposes such as antibody production. As discussed below, antibodies against HIN-2 are useful in a composition and method of the present invention.

Preferably, an isolated nucleic acid molecule of the present invention is produced using recombinant DNA technology (e.g., polymerase chain reaction (PCR) amplification, cloning) or chemical synthesis. Isolated nucleic acid molecules include natural nucleic acid molecules and homologues thereof, including, but not limited to, natural allelic variants and modified nucleic acid molecules in which nucleotides have been inserted, deleted, substituted, and/or inverted in such a manner that such modifications provide the desired effect on protein biological activity. Allelic variants and protein homologues (e.g., proteins encoded by nucleic acid homologues) have been discussed in detail above.

A nucleic acid molecule homologue can be produced using a number of methods known to those skilled in the art (see, for example, Sambrook et al., ibid.). For example, nucleic acid molecules can be modified using a variety of techniques including, but not limited to, classical mutagenesis techniques and recombinant DNA techniques, such as site-directed mutagenesis, chemical treatment of a nucleic acid molecule to induce mutations, restriction enzyme cleavage of a nucleic acid fragment, ligation of nucleic acid fragments, PCR amplification and/or mutagenesis of selected regions of a nucleic acid sequence, synthesis of oligonucleotide mixtures and ligation of mixture groups to “build” a mixture of nucleic acid molecules and combinations thereof. Nucleic acid molecule homologues can be selected from a mixture of modified nucleic acids by screening for the function of the protein encoded by the nucleic acid and/or by hybridization with a wild-type gene.

The minimum size of a nucleic acid molecule of the present invention is a size sufficient to encode a protein having the desired biological activity, or sufficient to form a probe or oligonucleotide primer that is capable of forming a stable hybrid with the complementary sequence of a nucleic acid molecule encoding the natural protein (e.g., under moderate, high or very high stringency conditions, and preferably under very high stringency conditions). As such, the size of a nucleic acid molecule of the present invention can be dependent on nucleic acid composition and percent homology or identity between the nucleic acid molecule and complementary sequence as well as upon hybridization conditions per se (e.g., temperature, salt concentration, and formamide concentration). The minimal size of a nucleic acid molecule that is used as an oligonucleotide primer or as a probe is typically at least about 12 to about 15 nucleotides in length if the nucleic acid molecules are GC-rich and at least about 15 to about 18 bases in length if they are AT-rich. There is no limit, other than a practical limit, on the maximal size of a nucleic acid molecule of the present invention, in that the nucleic acid molecule can include a portion of a protein-encoding sequence (e.g., a HIN-2-encoding sequence) or a nucleic acid sequence encoding a full-length protein, including any length fragment between about 12 and 279 nucleotides, in whole integers (e.g., 12, 13, 14, 15, 16 . . . 278, 279).

One embodiment of the present invention relates to a recombinant nucleic acid molecule which comprises the isolated nucleic acid molecule described above which is operatively linked to at least one transcription control sequence. More particularly, according to the present invention, a recombinant nucleic acid molecule typically comprises a recombinant vector and the isolated nucleic acid molecule as described herein. According to the present invention, a recombinant vector is an engineered (i.e., artificially produced) nucleic acid molecule that is used as a tool for manipulating a nucleic acid sequence of choice and/or for introducing such a nucleic acid sequence into a host cell. The recombinant vector is therefore suitable for use in cloning, sequencing, and/or otherwise manipulating the nucleic acid sequence of choice, such as by expressing and/or delivering the nucleic acid sequence of choice into a host cell to form a recombinant cell. Such a vector typically contains heterologous nucleic acid sequences, that is, nucleic acid sequences that are not naturally found adjacent to nucleic acid sequence to be cloned or delivered, although the vector can also contain regulatory nucleic acid sequences (e.g., promoters, untranslated regions) which are naturally found adjacent to nucleic acid sequences of the present invention or which are useful for expression of the nucleic acid molecules of the present invention (discussed in detail below). The vector can be either RNA or DNA, either prokaryotic or eukaryotic, and typically is a plasmid. The vector can be maintained as an extrachromosomal element (e.g., a plasmid) or it can be integrated into the chromosome of a recombinant host cell, although it is preferred if the vector remain separate from the genome for most applications of the invention. The entire vector can remain in place within a host cell, or under certain conditions, the plasmid DNA can be deleted, leaving behind the nucleic acid molecule of the present invention. An integrated nucleic acid molecule can be under chromosomal promoter control, under native or plasmid promoter control, or under a combination of several promoter controls. Single or multiple copies of the nucleic acid molecule can be integrated into the chromosome. A recombinant vector of the present invention can contain at least one selectable marker.

In one embodiment, a recombinant vector used in a recombinant nucleic acid molecule of the present invention is an expression vector. As used herein, the phrase “expression vector” is used to refer to a vector that is suitable for production of an encoded product (e.g., a protein of interest). In this embodiment, a nucleic acid sequence encoding the product to be produced (e.g., the HIN-2 protein or homologue thereof) is inserted into the recombinant vector to produce a recombinant nucleic acid molecule. The nucleic acid sequence encoding the protein to be produced is inserted into the vector in a manner that operatively links the nucleic acid sequence to regulatory sequences in the vector which enable the transcription and translation of the nucleic acid sequence within the recombinant host cell.

In another embodiment of the invention, the recombinant nucleic acid molecule comprises a viral vector. A viral vector includes an isolated nucleic acid molecule of the present invention integrated into a viral genome or portion thereof, in which the nucleic acid molecule is packaged in a viral coat that allows entrance of DNA into a cell. A number of viral vectors can be used, including, but not limited to, those based on alphaviruses, poxviruses, adenoviruses, herpesviruses, lentiviruses, adeno-associated viruses and retroviruses.

Typically, a recombinant nucleic acid molecule includes at least one nucleic acid molecule of the present invention operatively linked to one or more transcription control sequences. As used herein, the phrase “recombinant molecule” or “recombinant nucleic acid molecule” primarily refers to a nucleic acid molecule or nucleic acid sequence operatively linked to a transcription control sequence, but can be used interchangeably with the phrase “nucleic acid molecule”, when such nucleic acid molecule is a recombinant molecule as discussed herein. According to the present invention, the phrase “operatively linked” refers to linking a nucleic acid molecule to a transcription control sequence in a manner such that the molecule is able to be expressed when transfected (i.e., transformed, transduced, transfected, conjugated or conduced) into a host cell. Transcription control sequences are sequences which control the initiation, elongation, or termination of transcription. Particularly important transcription control sequences are those which control transcription initiation, such as promoter, enhancer, operator and repressor sequences. Suitable transcription control sequences include any transcription control sequence that can function in a host cell or organism into which the recombinant nucleic acid molecule is to be introduced.

Recombinant nucleic acid molecules of the present invention can also contain additional regulatory sequences, such as translation regulatory sequences, origins of replication, and other regulatory sequences that are compatible with the recombinant cell. In one embodiment, a recombinant molecule of the present invention, including those which are integrated into the host cell chromosome, also contains secretory signals (i.e., signal segment nucleic acid sequences) to enable an expressed protein to be secreted from the cell that produces the protein. Suitable signal segments include a signal segment that is naturally associated with the protein to be expressed or any heterologous signal segment capable of directing the secretion of the protein according to the present invention. In another embodiment, a recombinant molecule of the present invention comprises a leader sequence to enable an expressed protein to be delivered to and inserted into the membrane of a host cell. Suitable leader sequences include a leader sequence that is naturally associated with the protein, or any heterologous leader sequence capable of directing the delivery and insertion of the protein to the membrane of a cell.

According to the present invention, the term “transfection” is used to refer to any method by which an exogenous nucleic acid molecule (i.e., a recombinant nucleic acid molecule) can be inserted into a cell. The term “transformation” can be used interchangeably with the term “transfection” when such term is used to refer to the introduction of nucleic acid molecules into microbial cells or plants. In microbial systems, the term “transformation” is used to describe an inherited change due to the acquisition of exogenous nucleic acids by the microorganism and is essentially synonymous with the term “transfection.” However, in animal cells, transformation has acquired a second meaning which can refer to changes in the growth properties of cells in culture (described above) after they become cancerous, for example. Therefore, to avoid confusion, the term “transfection” is preferably used with regard to the introduction of exogenous nucleic acids into animal cells, and is used herein to generally encompass transfection of animal cells and transformation of plant cells and microbial cells, to the extent that the terms pertain to the introduction of exogenous nucleic acids into a cell. Therefore, transfection techniques include, but are not limited to, transformation, particle bombardment, electroporation, microinjection, lipofection, adsorption, infection and protoplast fusion.

One or more recombinant molecules of the present invention can be used to produce an encoded product (e.g., a HIN-2 protein, including homologues and a fusion or chimeric protein) of the present invention. In one embodiment, an encoded product is produced by expressing a nucleic acid molecule as described herein under conditions effective to produce the protein. A preferred method to produce an encoded protein is by transfecting a host cell with one or more recombinant molecules to form a recombinant cell. Suitable host cells to transfect include, but are not limited to, any bacterial, fungal (e.g., yeast), insect, plant or animal cell that can be transfected. Host cells can be either untransfected cells or cells that are already transfected with at least one other recombinant nucleic acid molecule.

In one embodiment, one or more protein(s) expressed by an isolated nucleic acid molecule of the present invention are produced by culturing a cell that expresses the protein (i.e., a recombinant cell or recombinant host cell) under conditions effective to produce the protein. In some instances, the protein may be recovered, and in others, the cell may be harvested in whole (e.g., for ex vivo administration), either of which can be used in a composition. A preferred cell to culture is any suitable host cell as described above. Effective culture conditions include, but are not limited to, effective media, bioreactor, temperature, pH and oxygen conditions that permit protein production and/or recombination. An effective medium refers to any medium in which a given host cell is typically cultured. Such medium typically comprises an aqueous medium having assimilable carbon, nitrogen and phosphate sources, and appropriate salts, minerals, metals and other nutrients, such as vitamins. Cells can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

Depending on the vector and host system used for production, resultant proteins of the present invention may either remain within the recombinant cell; be secreted into the culture medium; be secreted into a space between two cellular membranes; or be retained on the outer surface of a cell membrane. The phrase “recovering the protein” refers to collecting the whole culture medium containing the protein and need not imply additional steps of separation or purification. Proteins produced according to the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

Proteins of the present invention are preferably retrieved, obtained, and/or used in “substantially pure” form. As used herein, “substantially pure” refers to a purity that allows for the effective use of the protein in vitro, ex vivo or in vivo according to the present invention. For a protein to be useful in an in vitro, ex vivo or in vivo method according to the present invention, it is substantially free of contaminants, other proteins and/or chemicals that might interfere or that would interfere with its use in a method disclosed by the present invention, or that at least would be undesirable for inclusion with a HIN-2 protein (including homologues) when it is used in a method disclosed by the present invention. Such methods include antibody production, agonist/antagonist identification assays, preparation of therapeutic compositions, administration in a therapeutic composition, and all other methods disclosed herein. Preferably, a “substantially pure” protein, as referenced herein, is a protein that can be produced by any method (i.e., by direct purification from a natural source, recombinantly, or synthetically), and that has been purified from other protein components such that the protein comprises at least about 80% weight/weight of the total protein in a given composition (e.g., the HIN-2 protein is about 80% of the protein in a solution/composition/buffer), and more preferably, at least about 85%, and more preferably at least about 90%, and more preferably at least about 91%, and more preferably at least about 92%, and more preferably at least about 93%, and more preferably at least about 94%, and more preferably at least about 95%, and more preferably at least about 96%, and more preferably at least about 97%, and more preferably at least about 98%, and more preferably at least about 99%, weight/weight of the total protein in a given composition.

It will be appreciated by one skilled in the art that use of recombinant DNA technologies can improve control of expression of transfected nucleic acid molecules by manipulating, for example, the number of copies of the nucleic acid molecules within the host cell, the efficiency with which those nucleic acid molecules are transcribed, the efficiency with which the resultant transcripts are translated, and the efficiency of post-translational modifications. Additionally, the promoter sequence might be genetically engineered to improve the level of expression as compared to the native promoter. Recombinant techniques useful for controlling the expression of nucleic acid molecules include, but are not limited to, integration of the nucleic acid molecules into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgarno sequences), modification of nucleic acid molecules to correspond to the codon usage of the host cell, and deletion of sequences that destabilize transcripts.

As discussed above, embodiments of the invention relate to modified HIN-2 receptors (e.g., HIN-2 receptor homologues). Knowing the identity of a receptor for HIN-2 allows one of skill in the art to modify the receptor in a manner that the ability of the receptor to interact with HIN-2 and be activated by HIN-2 or other ligands can be regulated. Therefore, yet another embodiment of the present invention relates to an isolated HIN-2 receptor homologue, and preferably, an isolated MARCO homologue. The homologue comprises an amino acid sequence that is at least about 50% identical to SEQ ID NO:6 and is less than 100% identical to SEQ ID NO:6 or SEQ ID NO:8. Preferably, the receptor homologue is at least about 55% identical, or at least about 60% identical, or at least about 65% identical, or at least about 70% identical, or at least about 75% identical, or at least about 80% identical, or at least about 85% identical, or at least about 90% identical, or at least about 95% identical, or at least about 96% identical, or at least about 97% identical, or at least about 98% identical, or at least about 99% identical to SEQ ID NO:6, over the full length of any of such sequences. In another embodiment, the receptor homologue is less than about 99% identical to the above-identified amino acid sequences, and in another embodiment, is less than is less than 98% identical to any of the above-identified amino acid sequences, and in another embodiment, is less than 97% identical to any of the above-identified amino acid sequences, and in another embodiment, is less than 96% identical to any of the above-identified amino acid sequences, and in another embodiment, is less than 95% identical to any of the above-identified amino acid sequences, and in another embodiment, is less than 94% identical to any of the above-identified amino acid sequences, and in another embodiment, is less than 93% identical to any of the above-identified amino acid sequences, and in another embodiment, is less than 92% identical to any of the above-identified amino acid sequences, and in another embodiment, is less than 91% identical to any of the above-identified amino acid sequences, and in another embodiment, is less than 90% identical to any of the above-identified amino acid sequences, and so on, in increments of whole integers.

Preferably, an isolated MARCO homologue ofthe present invention has MARCO biological activity. MARCO biological activity has been described in detail previously herein, and can include any of the functions of a MARCO receptor, and also MARCO transcription, MARCO translation, MARCO phosphorylation, and NFκB activation.

In one embodiment, a MARCO homologue is a soluble MARCO receptor. In another embodiment, a MARCO homologue binds to HIN-2, and in one aspect of this embodiment, such a MARCO homologue is not activated by the binding of HIN-2 as would be a wild-type MARCO receptor. In another embodiment, a MARCO homologue does not bind to HIN-2, or binds with less affinity to HIN-2 as compared to the wild-type receptor. In yet another embodiment, the MARCO homologue activates NF-κB in a cell expressing the homologue through the IL-1R pathway.

The present invention also includes a fusion protein that includes a MARCO—containing domain (including a homologue of MARCO) attached to one or more fusion segments (e.g., an immunoglobulin constant region), fusion segments having been described above.

The present invention also includes a mimetic of a MARCO receptor. The term “mimetic” has been defined above.

Yet another embodiment of the present invention relates to an isolated nucleic acid molecule comprising a nucleic acid sequence encoding any of the above-identified MARCO homologues. Recombinant nucleic acid molecules, viruses, and cells have been described in detail above with regard to HIN-2 proteins and that discussion is applied here to recombinant nucleic acid molecules, viruses and cells comprising nucleic acid molecules encoding MARCO homologues.

Another embodiment of the present invention relates to an isolated binding agent selected from an antibody, an antigen binding fragment, or a binding partner. The binding agent selectively binds to an amino acid sequence selected from SEQ ID NO:2, amino acids 22-93 of SEQ ID NO:2, SEQ ID NO:4, or amino acids 22-91 of SEQ ID NO:4 or to a receptor for HIN-2, such as MARCO (SEQ ID NO:6 or SEQ ID NO:8). Such an binding agent can selectively bind to any HIN-2 protein or receptor therefor, including fragments of such receptors. According to the present invention, the phrase “selectively binds to” refers to the ability of an antibody, antigen binding fragment or binding partner of the present invention to preferentially bind to specified proteins. More specifically, the phrase “selectively binds” refers to the specific binding of one protein to another (e.g., an antibody, fragment thereof, or binding partner to an antigen), wherein the level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen binding fragment thereof in the absence of the antigen is considered to be background. Binding can be measured using a variety of methods standard in the art including enzyme immunoassays (e.g., ELISA), immunoblot assays, etc.).

Antibodies are characterized in that they comprise immunoglobulin domains and as such, they are members of the immunoglobulin superfamily of proteins. An antibody of the invention includes polyclonal and monoclonal antibodies, divalent and monovalent antibodies, bi- or multi-specific antibodies, serum containing such antibodies, antibodies that have been purified to varying degrees, and any functional equivalents of whole antibodies. Isolated antibodies of the present invention can include serum containing such antibodies, or antibodies that have been purified to varying degrees. Whole antibodies of the present invention can be polyclonal or monoclonal. Alternatively, functional equivalents of whole antibodies, such as antigen binding fragments in which one or more antibody domains are truncated or absent (e.g., Fv, Fab, Fab′, or F(ab)₂ fragments), as well as genetically-engineered antibodies or antigen binding fragments thereof, including single chain antibodies or antibodies that can bind to more than one epitope (e.g., bi-specific antibodies), or antibodies that can bind to one or more different antigens (e.g., bi- or multi-specific antibodies), may also be employed in the invention.

Genetically engineered antibodies of the invention include those produced by standard recombinant DNA techniques involving the manipulation and re-expression of DNA encoding antibody variable and/or constant regions. Particular examples include, chimeric antibodies, where the V_{H} and/or V_{L} domains of the antibody come from a different source to the remainder of the antibody, and CDR grafted antibodies (and antigen binding fragments thereof), in which at least one CDR sequence and optionally at least one variable region framework amino acid is (are) derived from one source and the remaining portions of the variable and the constant regions (as appropriate) are derived from a different source. Construction of chimeric and CDR-grafted antibodies are described, for example, in European Patent Applications: EP-A 0194276, EP-A 0239400, EP-A 0451216 and EP-A 0460617.

Generally, in the production of an antibody, a suitable experimental animal, such as, for example, but not limited to, a rabbit, a sheep, a hamster, a guinea pig, a mouse, a rat, or a chicken, is exposed to an antigen against which an antibody is desired. Typically, an animal is immunized with an effective amount of antigen that is injected into the animal. An effective amount of antigen refers to an amount needed to induce antibody production by the animal. The animal's immune system is then allowed to respond over a pre-determined period of time. The immunization process can be repeated until the immune system is found to be producing antibodies to the antigen. In order to obtain polyclonal antibodies specific for the antigen, serum is collected from the animal that contains the desired antibodies (or in the case of a chicken, antibody can be collected from the eggs). Such serum is useful as a reagent. Polyclonal antibodies can be further purified from the serum (or eggs) by, for example, treating the serum with ammonium sulfate.

Monoclonal antibodies may be produced according to the methodology of Kohler and Milstein (Nature 256:495-497, 1975). For example, B lymphocytes are recovered from the spleen (or any suitable tissue) of an immunized animal and then fused with myeloma cells to obtain a population of hybridoma cells capable of continual growth in suitable culture medium. Hybridomas producing the desired antibody are selected by testing the ability of the antibody produced by the hybridoma to bind to the desired antigen.

The invention also extends to non-antibody polypeptides, sometimes referred to as binding partners, that have been designed to bind specifically to, and either activate or inhibit as appropriate, a HIN-2 protein or receptor thereof of the invention. Examples of the design of such polypeptides, which possess a prescribed ligand specificity are given in Beste et al. (Proc. Natl. Acad. Sci. 96:1898-1903, 1999), incorporated herein by reference in its entirety.

In one embodiment, a binding agent of the invention is immobilized on a substrate such as: artificial membranes, organic supports, biopolymer supports and inorganic supports such as for use in a screening assay.

Having described HIN-2 proteins and HIN-2 receptors in detail, various aspects of the present invention related to the use of HIN-2 and its receptor will be described. In one embodiment, the present invention also includes methods which use nucleic acid sequences encoding HIN-2, HIN-2 proteins (including HIN-2 homologues), HIN-2 antibodies, HIN-2 receptors (including HIN-2 receptor homologues) and HIN-2 receptor antibodies as therapeutic reagents, screening tools and/or diagnostic tools. As such, one embodiment of the present invention relates to a method to identify regulators of HIN-2 or a HIN-2 receptor by identifying putative regulatory compounds which increase, decrease or mimic the expression and/or biological activity of HIN-2 proteins or nucleic acid molecules and/or HIN-2 receptor proteins or nucleic acid molecules, including downstream events associated with the activity of HIN-2 and/or HIN-2 receptors.

Such methods can be cell-free or a cell-based assays. In one embodiment, a method to identify a compound that regulates HIN-2 expression or biological activity is provided. The method includes the steps of: (a) contacting a HIN-2 protein, a biologically active fragment thereof, or a receptor-binding fragment thereof, with a putative regulatory compound; and (b) detecting whether the putative regulatory compound binds to or regulates the expression or activity of the HIN-2 protein, biologically active fragment or receptor-binding fragment as compared to prior to contact with the compound. A compound that binds to the protein and increases or decreases the expression or activity of the protein, as compared to the protein in the absence of the compound, indicates that the putative regulatory compound is a regulator of HIN-2.

In one aspect, the step of detecting comprises detecting whether HIN-2 expression or activity is regulated by contacting cells or tissue with the putative regulatory compound and measuring HIN-2 expression in the cells or tissue. In another aspect, the step of detecting comprises detecting whether HIN-2 expression or activity is regulated during allergic inflammation conditions in the presence of the putative regulatory compound as compared to in the absence of the putative regulatory compound. In another aspect, the step of detecting comprises detecting whether HIN-2 expression or activity is regulated during proinflammatory conditions in the presence of the putative regulatory compound as compared to in the absence of the putative regulatory compound. In another aspect, the step of detecting comprises detecting whether the putative regulatory compound inhibits, enhances, or competes with the binding of HIN-2 to a HIN-2 receptor (e.g., MARCO). In yet another aspect, the step of detecting comprises detecting whether the putative regulatory compound inhibits, enhances, or competes with the binding of HIN-2 to a ligand selected from the group consisting of lipopolysaccharide (LPS), apolipoprotein AI, a bacterium, and/or a yeast. In addition, one can detect whether the putative regulatory compound regulates inflammation in a cell, tissue, or non-human animal, including, but not limited to, in the lung cells or lung tissue of an animal. One can also further detect whether the putative regulatory compound regulates the level of high density lipoproteins or low density lipoproteins in a mammal. Various aspects of this method are described in more detail below.

In another related embodiment, the invention provides a method to identify a HIN-2 homologue that regulates HIN-2 biological activity. In this embodiment, the HIN-2 homologue is effectively the putative regulatory compound to be evaluated for activity. This method comprising detecting whether a putative HIN-2 homologue has at least one biological activity selected from the group consisting of: (a) binds to a HIN-2 receptor (e.g., MARCO) or to a HIN-2-binding portion of a HIN-2 receptor; (b) increases the activity of a HIN-2 receptor; (c) binds to a bacterial cell; (d) binds to a yeast; (e) binds to a lipopolysaccharide (LPS); (f) binds to apolipoprotein AI; (g) regulates phagocytosis of a bacterium by a macrophage as compared to in the absence of the putative HIN-2 homologue; (h) regulates inflammation as compared to in the absence of the putative HIN-2 homologue; (i) regulates airway hyperresponsiveness as compared to in the absence of the putative HIN-2 homologue; (j) regulates innate immune responses in a site in a mammal as compared to in the absence of the putative HIN-2 homologue. HIN-2 homologues according to the invention have been described in detail above.

In one aspect of these methods, the methods can include a step of contacting a HIN-2 receptor with a putative regulatory compound (including a putative HIN-2 homologue agonist or antagonist); (b) contacting the HIN-2 receptor with a HIN-2 protein; and (c) detecting whether the HIN-2 protein is capable of binding to the receptor in the presence of the putative regulatory compound as compared to in the absence of the putative regulatory compound. Alternatively, the method can include the steps of: (a) contacting a HIN-2 protein with a putative regulatory compound; (b) contacting the HIN-2 protein with a HIN-2 receptor; and, (c) detecting whether the HIN-2 protein is capable of binding to the receptor in the presence of the putative regulatory compound as compared to in the absence of the putative regulatory compound. Preferably, step (a) is performed prior to step (b) in each of these embodiments. In one aspect, an additional step can be performed prior to step (b) to determine whether the putative compound regulates (binds to and/or activates) HIN-2 or HIN-2 receptor activity directly. In these embodiments, the HIN-2 protein can be provided as a substantially purified protein or in a cell-free extract (e.g., the membrane form separate from cell membranes). The HIN-2 receptor (e.g., MARCO) can be provided as a soluble protein or otherwise cell-free (e.g., in the absence of cell membranes); expressed by a cell, such as a monocyte, macrophage, tumor cell, or other cell capable of expressing HIN-2 receptor; or in a cell lysate. In other aspects where binding of HIN-2 or a homologue thereof is detected, instead of using a HIN-2 receptor, one can use another ligand of HIN-2 in the assay, such as a bacterium, a portion of a bacterium (e.g., lysate, cell wall, etc.), a yeast, a portion of a yeast, LPS, or Apolipoprotein A-I.

In these embodiments, reduced binding of HIN-2 to its receptor or another ligand in the presence of the putative regulatory compound as compared to in the absence of the compound indicates that the putative regulatory compound is an inhibitor of HIN-2 and HIN-2 receptor/other ligand interaction. The compound can be further tested (or alternatively tested), if desired, in a cell-based assay to determine whether the compound binds to HIN-2 or to HIN-2 receptor/other ligand and also whether the compound inhibits or enhances the biological activity of the HIN-2 receptor, as determined by a reduction biological activity associated with the receptor as previously described herein. Such further steps will help detect the mode of action of the compound and whether it might be an agonist or antagonist of HIN-2 or its receptor. Similarly, in this embodiment, increased binding of HIN-2 to its receptor or other ligand in the presence of the putative regulatory compound as compared to in the absence of the compound indicates that the putative regulator is an enhancer of HIN-2 and HIN-2 receptor/other ligand interaction. If no change in the binding of HIN-2 to its receptor or other ligand is detected in the presence of the putative regulatory compound as compared to in the absence of the compound, one can conclude that the compound does not appear to affect the binding of HIN-2 to its receptor or other ligand, although biological activity impact can still be evaluated. In this instance, a cell-based assay can be used to detect whether the putative regulatory compound increases or decreases the biological activity of HIN-2 or a HIN-2 receptor.

In yet another aspect, if a putative HIN-2 protein homologue is to be evaluated (e.g., the homologue is the putative regulatory compound to be tested as being an agonist or antagonist of HIN-2), the HIN-2 homologue may tested for one of the above-identified activities (e.g., binding or biological activities), without the need for the presence of HIN-2, except as a positive or negative control.

As used herein, the term “putative” refers to compounds having an unknown or previously unappreciated regulatory activity in a particular process. As such, the term “identify” is intended to include all compounds, the usefulness of which as a regulatory compound of the interaction between HIN-2 and HIN-2 receptor or other ligand for the purposes of regulating processes associated with innate immunity or inflammation is determined by a method of the present invention.

The methods of the present invention include contacting components chosen from: HIN-2 protein, a HIN-2 homologue, a HIN-2 receptor and/or a putative regulatory compound with one another to detect binding of one component to another or to detect the effect of the contact on expression and/or biological activity of one or more of the components. The step of contacting can be performed by any suitable method, depending on how the HIN-2, HIN-2 receptor, HIN-2 homologue, and/or putative regulatory compound are provided. For example, cells expressing HIN-2 receptor can be grown in liquid culture medium or grown on solid medium in which the liquid medium or the solid medium contains the compound to be tested in the presence or absence of HIN-2. In addition, as described above, the liquid or solid medium contains components necessary for cell growth, such as assimilable carbon, nitrogen and micro-nutrients. Cell lysates can be combined with other cell lysates and/or the compound to be tested in any suitable medium. In another embodiment, the HIN-2 protein, HIN-2 homologue, and/or the HIN-2 receptor and/or cell lysates containing such proteins can be immobilized on a substrate such as: artificial membranes, organic supports, biopolymer supports and inorganic supports. The protein can be immobilized on the solid support by a variety of methods including adsorption, cross-linking (including covalent bonding), and entrapment. Adsorption can be through van del Waal's forces, hydrogen bonding, ionic bonding, or hydrophobic binding. Exemplary solid supports for adsorption immobilization include polymeric adsorbents and ion-exchange resins. Solid supports can be in any suitable form, including in a bead form, plate form, or well form. The putative regulatory compound can be contacted with the immobilized protein by any suitable method, such as by flowing a liquid containing the compound over the immobilized protein.

The present methods involve contacting cells with the compound being tested for a sufficient time to allow for interaction with, activation of or inhibition of the HIN-2 protein or HIN-2 receptor by the compound. The period of contact with the compound being tested can be varied depending on the result being measured, and can be determined by one of skill in the art. For example, for binding assays, a shorter time of contact with the compound being tested is typically suitable, than when activation is assessed. As used herein, the term “contact period” refers to the time period during which the proteins are in contact with the compound being tested and/or the time period during which the HIN-2 protein or the HIN-2 receptor are in contact (or in a condition where contact is possible) with each other. The term “incubation period” refers to the entire time during which, for example, cells are allowed to grow prior to evaluation, and can be inclusive of the contact period. Thus, the incubation period includes all of the contact period and may include a further time period during which the compound being tested is not present but during which growth is continuing (in the case of a cell based assay) prior to scoring. The incubation time for growth of cells can vary but is sufficient to allow for the binding of the HIN-2 receptor, activation of the receptor, and/or inhibition of the receptor. It will be recognized that shorter incubation times are preferable because compounds can be more rapidly screened. A preferred incubation time is between about 1 minute to about 48 hours.

The conditions under which the cell or cell lysate of the present invention is contacted with a putative regulatory compound and/or with other cells or cell lysates, such as by mixing, are any suitable culture or assay conditions and includes an effective medium in which the cell can be cultured or in which the cell lysate can be evaluated in the presence and absence of a putative regulatory compound. Similarly, the conditions under which HIN-2 and/or HIN-2 receptors are contacted with a putative regulatory compound and/or with each other are any suitable assay conditions, such as by immobilization of the ligand or receptor on a substrate in conditions under which the ligand and/or receptor can contact the putative regulatory compound prior to, simultaneously with, or after contact of the ligand and receptor with each other.

Cells of the present invention can be cultured in a variety of containers including, but not limited to, tissue culture flasks, test tubes, microtiter dishes, and petri plates. Culturing is carried out at a temperature, pH and carbon dioxide content appropriate for the cell. Such culturing conditions are also within the skill in the art. Acceptable protocols to contact a cell with a putative regulatory compound in an effective manner include the number of cells per container contacted, the concentration of putative regulatory compound(s) administered to a cell, the incubation time of the putative regulatory compound with the cell, and the concentration of compound administered to a cell. Determination of such protocols can be accomplished by those skilled in the art based on variables such as the size of the container, the volume of liquid in the container, the type of cell being tested and the chemical composition of the putative regulatory compound (i.e., size, charge etc.) being tested. A preferred amount of putative regulatory compound(s) comprises between about 1 nM to about 10 mM of putative regulatory compound(s) per well of a 96-well plate.

Suitable cells for use with the present invention include any cell that endogenously expresses a HIN-2 receptor or a HIN-2 protein as disclosed herein (such as a lung cell for HIN-2), or which has been transfected with and expresses recombinant HIN-2 receptor or HIN-2 protein as disclosed herein (such as 293 cells, COS cells, CHO cells, fibroblasts, etc., genetically engineered to express HIN-2 or HIN-2 receptor). In one embodiment, host cells genetically engineered to express a functional receptor that responds to activation by HIN-2 protein can be used as an endpoint in the assay; e.g., as measured by a chemical, physiological, biological, or phenotypic change, induction of a host cell gene or a reporter gene, change in cAMP levels, adenylyl cyclase activity, host cell G protein activity, host cell kinase activity, proliferation, differentiation, etc. Cells for use with the present invention include mammalian, invertebrate, plant, insect, fungal, yeast and bacterial cells. Preferred cells include mammalian cells. Preferred mammalian cells include primate, non-human primate, mouse and rat, with human cells being preferred. Preferably, the test cell (host cell) should express a functional HIN-2 receptor that gives a significant response to interaction with a HIN-2 protein that is known to bind to and activate the HIN-2 receptor, preferably greater than 2, 5, or 10-fold induction over background.

The HIN-2 protein can be contacted with the HIN-2 receptor or other ligand (or the cell expressing such receptor) prior to, simultaneous with, or after contact of the putative regulatory compound with the cell, depending on how the assay is to be evaluated, and depending on whether activation or inhibition of the receptor and/or cell expressing the receptor is to be evaluated. In one embodiment, the HIN-2 protein is contacted with the HIN-2 receptor or other ligand after the cell is contacted with the putative regulatory compound so that the test compound can be evaluated for its ability to inhibit activation of the receptor by the HIN-2 protein. In another embodiment, when binding is to be evaluated, the HIN-2 protein can be contacted with the HIN-2 receptor or other ligand at the same time as the test compound. Preferably, the HIN-2 protein is contacted with the cell/HIN-2 receptor or other ligand in the presence and absence of the test compound for a controlled assay. In some embodiments, a HIN-2 homologue is contacted with a HIN-2 receptor or other ligand in the absence of HIN-2 or other compounds.

As discussed above, the step of detecting whether a putative regulatory compound binds to, activates and/or inhibits the interaction between HIN-2 and its receptor can be performed by any suitable method, including, but not limited to measurement of HIN-2 receptor transcription; measurement of HIN-2 receptor translation; measurement of phosphorylation of the HIN-2 receptor; measurement of HIN-2 receptor or other ligand binding to HIN-2, to a HIN-2 homologue or to a putative regulatory compound; measurement of HIN-2 receptor translocation within a cell; measurement of NFκB activation by HIN-2 receptor; measuring binding to bacteria or phagocytosis of bacteria by macrophages; measuring binding to yeast or phagocytosis of yeast, by measuring chemotaxis of macrophages to a site of inflammation; measuring markers of inflammation (e.g., cytokine production and types, cellular infiltration into a site, production of other inflammatory mediators); measurement of airway hyperresponsiveness; or measuring markers of activation of the innate immune responses in a site (e.g., chemotaxis, influx of cell types, production of is monocyte/macrophage/neutrophil activation markers or activation products). Such methods are known in the art, and include a variety of binding assays, western blotting, immunocytochemistry, flow cytometry, other immunological based assays, phosphorylation assays, kinase assays, immunofluorescence microscopy, RNA assays, immunoprecipitation, cytokine assays, evaluation of cell morphology, in situ hybridization, and other biological assays. Binding assays include BIAcore machine assays, immunoassays such as enzyme linked immunoabsorbent assays (ELISA) and radioimmunoassays (RIA), or determination of binding by monitoring the change in the spectroscopic or optical properties of the proteins through fluorescence, UV absorption, circular dichrosim, or nuclear magnetic resonance (NMR). Assays for evaluating the biological activity of MARCO, for example, are described in the publication discussed above and incorporated by reference.

As disclosed above, the present methods also make use of non-cell based assay systems to identify compounds that can regulate the interaction between HIN-2 and HIN-2 receptor or the activity of one or the other component. For example, isolated membranes may be used to identify compounds that interact with the HIN-2 receptor being tested. Membranes can be harvested from cells expressing HIN-2 receptors by standard techniques and used in an in vitro binding assay. ¹²⁵I-labeled ligand (e.g., 125I-labeled HIN-2) is bound to the membranes and assayed for specific activity; specific binding is determined by comparison with binding assays performed in the presence of excess unlabeled ligand. Membranes are typically incubated with labeled ligand in the presence or absence of test compound (i.e., a putative regulatory compound). Compounds that bind to the receptor and compete with labeled ligand for binding to the membranes reduced the signal compared to the vehicle control samples.

Alternatively, soluble HIN-2 receptors or HIN-2 proteins may be recombinantly expressed and utilized in non-cell based assays to identify compounds that bind to HIN-2 receptors or HIN-2 proteins, respectively. Recombinantly expressed HIN-2 receptor or HIN-2 polypeptides or fusion proteins containing one or more extracellular domains of HIN-2 receptor or HIN-2 can be used in the non-cell based screening assays. Alternatively, peptides corresponding to a cytoplasmic domain of the HIN-2 receptor or fusion proteins containing a cytoplasmic domain of the HIN-2 receptor can be used in non-cell based assay systems to identify compounds that bind to the cytoplasmic portion of the HIN-2 receptor; such compounds may be useful to modulate the signal transduction pathway of the HIN-2 receptor. In non-cell based assays the recombinantly expressed HIN-2 receptor is attached to a solid substrate such as a test tube, microtiter well or a column, by means well known to those in the art. The test compounds are then assayed for their ability to bind to the HIN-2 receptor.

As discussed above, in vitro cell based assays may be designed to screen for compounds that regulate HIN-2 expression or HIN-2 receptor expression at either the transcriptional or translational level. For example, one embodiment of the invention relates to a method to identify a compound that regulates the expression of Hin-2, comprising: (a) contacting a putative regulatory compound with a recombinant host cell that expresses a recombinant nucleic acid, molecule encoding HIN-2 or a recombinant host cell that has been transfected with a nucleic acid sequence comprising a Hin-2 regulatory region operatively linked to a reporter nucleic acid sequence; and (b) detecting whether the putative regulatory compound regulates expression of the recombinant nucleic acid molecule encoding HIN-2 or the reporter nucleic acid sequence as compared to prior to contact with the compound. Compounds that regulate expression of the recombinant nucleic acid molecule encoding HIN-2 or the reporter nucleic acid sequence, as compared to in the absence of the compound, indicates that the putative regulatory compound is a regulator of Hin-2 expression. More particularly, in one embodiment, a nucleic acid sequence encoding a reporter molecule can be linked to a regulatory element of Hin2 or the HIN-2 receptor gene and used in appropriate intact cells, cell extracts or lysates to identify compounds that modulate HIN-2 expression or HIN-2 receptor gene expression. respectively. Appropriate cells or cell extracts can be prepared, if desired, from any cell type that normally expresses HIN-2 or a HIN-2 receptor gene, thereby ensuring that the cell extracts contain the transcription factors required for in vitro or in vivo transcription. The screen can be used to identify compounds that modulate the expression of the reporter construct. In such screens, the level of reporter gene expression is determined in the presence of the test compound and compared to the level of expression in the absence of the test compound.

According to the present invention, the method can include the step of detecting the expression of at least one, and preferably more than one, of the downstream genes that are regulated by the interaction between HIN-2 and a HIN-2 receptor, such as MARCO. As used herein, the term “expression”, when used in connection with detecting the expression of a downstream gene of the present invention, can refer to detecting transcription of the gene and/or to detecting translation of the gene. To detect expression of a downstream gene refers to the act of actively determining whether a gene is expressed or not. This can include determining whether the gene expression is upregulated as compared to a control, downregulated as compared to a control, or unchanged as compared to a control. Therefore, the step of detecting expression does not require that expression of the gene actually is upregulated or downregulated, but rather, can also include detecting that the expression of the gene has not changed (i.e., detecting no expression of the gene or no change in expression of the gene). Expression of transcripts and/or proteins is measured by any of a variety of known methods in the art. For RNA expression, methods include but are not limited to: extraction of cellular mRNA and northern blotting using labeled probes that hybridize to transcripts encoding all or part of one or more of the genes of this invention; amplification of mRNA expressed from one or more of the genes of this invention using gene-specific primers and reverse transcriptase—polymerase chain reaction, followed by quantitative detection of the product by any of a variety of means; extraction of total RNA from the cells, which is then labeled and used to probe cDNAs or oligonucleotides encoding all or part of the genes of this invention, arrayed on any of a variety of surfaces. The term “quantifying” or “quantitating” when used in the context of quantifying transcription levels of a gene can refer to absolute or to relative quantification. Absolute quantification may be accomplished by inclusion of known concentration(s) of one or more target nucleic acids and referencing the hybridization intensity of unknowns with the known target nucleic acids (e.g. through generation of a standard curve). Alternatively, relative quantification can be accomplished by comparison of hybridization signals between two or more genes, or between two or more treatments to quantify the changes in hybridization intensity and, by implication, transcription level.

In any of the above-described methods of the present invention, the HIN-2 protein can be any HIN-2 protein described herein, including homologues that are capable of binding to and/or activating the receptor in the absence of the putative regulatory compound. Similarly, the HIN-2 receptor can be any HIN-2 receptor described herein, including homologues that are capable of binding to HIN-2 and/or being activated by a HIN-2 protein in the absence of a putative regulatory compound.

Another embodiment of the present invention relates to a method to diagnose a disorder associated with HIN-2 expression or biological activity. The method includes the steps of detecting expression or biological activity of HIN-2 or a gene encoding HIN-2 in a tissue of a patient suspected of having the disorder, and comparing the expression or biological activity to a control, wherein a difference in the expression or biological activity of HIN-2 or a gene encoding HIN-2 in a tissue of the patient as compared to the control indicates a positive diagnosis of a disorder associated with HIN-2. The disorder can be any disorder that is associated with inflammation (Th1- or Th2-based) or an innate immune response, and includes, but is not limited to, a lung disorder, a disorder associated with allergic inflammation, a disorder is associated with microbial infection, an autoimnmune disease, a cancer, or any non-infectious inflammation. In one embodiment, the disorder includes, but is not limited to, asthma, interstitial lung disease, cystic fibrosis, rheumatoid arthritis, reactive arthritis, lung cancer, bacterial infection (e.g., Salmonella infection), yeast infection, or spondylarthropathy.

The terms “diagnose”, “diagnosis”, “diagnosing” and variants thereof refer to the identification of a disease or condition on the basis of its signs and symptoms. As used herein, a “positive diagnosis” indicates that the disease or condition, or a potential for developing the disease or condition, has been identified. In contrast, a “negative diagnosis” indicates that the disease or condition, or a potential for developing the disease or condition, has not been identified.

According to the present invention, the term “cell sample” can be used generally to refer to a sample of any type which contains cells to be evaluated by the present method, including but not limited to, a sample of isolated cells, a tissue sample and/or a bodily fluid sample. According to the present invention, a sample of isolated cells is a specimen of cells, typically in suspension or separated from connective tissue which may have connected the cells within a tissue in vivo, which have been collected from an organ, tissue or fluid by any suitable method which results in the collection of a suitable number of cells for evaluation by the method of the present invention. The cells in the cell sample are not necessarily of the same type, although purification methods can be used to enrich for the type of cells which are preferably evaluated. Cells can be obtained, for example, by scraping of a tissue, processing of a tissue sample to release individual cells, or isolation from a bodily fluid. A tissue sample, although similar to a sample of isolated cells, is defined herein as a section of an organ or tissue of the body which typically includes several cell types and/or cytoskeletal structure which holds the cells together. One of skill in the art will appreciate that the term “tissue sample” may be used, in some instances, interchangeably with a “cell sample”, although it is preferably used to designate a more complex structure than a cell sample. A tissue sample can be obtained by a biopsy, for example, including by cutting, slicing, or a punch. A bodily fluid sample, like the tissue sample, contains the cells to be evaluated for HIN-2 or Hin-2 expression or biological activity, and is a fluid obtained by any method suitable for the particular bodily fluid to be sampled. Bodily fluids suitable for sampling include, but are not limited to, blood, mucous, seminal fluid, saliva, breast milk, bile and urine.

Methods suitable for detecting Hin-2 transcription or HIN-2 translation have been described elsewhere herein and include any suitable method for detecting and/or measuring mRNA levels or protein levels from a cell, cell extract or tissue. Methods for detecting transcription include, but are not limited to: polymerase chain reaction (PCR), reverse transcriptase PCR (RT-PCR), in situ hybridization, Northern blot, sequence analysis, and detection of a reporter gene. Such methods for detection of transcription levels are well known in the art, and many of such methods are described in Sambrook et al., Molecular Cloning: A Laboratory Manual, Cold Spring Harbor Labs Press, 1989 and/or in Glick et al., Molecular Biotechnology: Principles and Applications of Recombinant DNA, ASM Press, 1998; Sambrook et al., ibid., and Glick et al., ibid. are incorporated by reference herein in their entireties. Methods suitable for the detection of HIN-2 protein include, but are not limited to, immunoblot (e.g., Western blot), enzyme-linked immunosorbant assay (ELISA), radioimmunoassay (RIA), immunoprecipitation, immunohistochemistry and immunofluorescence. Such methods are well known in the art.

The method of the present invention includes a step of comparing the level of HIN-2 or Hin-2 expression or biological activity detected in step (a) to a baseline level of HIN-2 or Hin-2 expression or biological activity. According to the present invention, a “baseline level” is a control level, and in some embodiments, a normal level of HIN-2 or Hin-2 expression or activity against which a test level of HIN-2 or Hin-2 expression or biological activity (i.e., in the patient sample) can be compared. The method for establishing a baseline level of HIN-2 or Hin-2 expression or activity is selected based on the sample type, the tissue or organ from which the sample is obtained, the status of the patient to be evaluated, and the focus or goal of the assay (e.g., diagnosis, staging, monitoring). Preferably, the method is the same method that will be used to evaluate the sample in the patient. Baseline levels can be established using an autologous control sample obtained from the patient, an autologous level in a previous sample from the same patient, or using control samples that were obtained from a population of matched individuals.

Some embodiments of the present invention include a composition comprising HIN-2 or a regulator thereof for diagnostic, screening or therapeutic purposes. Therefore, another embodiment of the invention relates to a composition comprising a compound selected from: (i) an isolated HIN-2 protein, fragment or homologue thereof; (ii) a HIN-2 agonist or antagonist compound other than a protein HIN-2 homologue; (iii) an isolated antibody that specifically binds to a HIN-2 protein of (i); (iv) an isolated HIN-2 receptor, fragment, or homologue thereof; (v) a HIN-2 receptor agonist or antagonist other than a protein HIN-2 receptor homologue; or, (vi) an isolated antibody that specifically binds to a HIN-2 receptor of (iv). The composition typically also includes a pharmaceutically acceptable carrier. In this aspect of the present invention, an isolated HIN-2 protein can be any of the HIN-2 proteins previously described herein, including, but not limited to, a wild-type HIN-2 protein, a HIN-2 protein homologue, a soluble HIN-2 protein, and/or a HIN-2 fusion protein. Similarly, an isolated HIN-2 receptor can be any of the HIN-2 receptor proteins previously described herein, including, but not limited to, a wild-type HIN-2 receptor, a HIN-2 receptor homologue, a soluble HIN-2 receptor, and/or a HIN-2 receptor fusion protein. An isolated antibody that selectively binds to a HIN-2 protein or a HIN-2 receptor has also been described above. Agonists and antagonists of HIN-2 and HIN-2 receptors have also been described above. In one embodiment, a composition of the present invention can include nucleic acid molecules encoding HIN-2 and/or a HIN-2 receptor, and/or a mimetic of HIN-2 or a HIN-2 receptor. In one embodiment, a composition of the present invention includes a combination of at least two of any of the above-identified compounds.

Compositions of the present invention are useful for regulating biological processes and particularly, processes associated with innate immunity, inflammation, and in one embodiment, cancer. Such processes typically involve cells of the innate immune system, including, but not limited to, monocytes, macrophages, and neutrophils. In particular, compositions of the present invention are useful for regulating the interaction between HIN-2 and its receptor (HIN-2 receptor, such as MARCO) or between HIN-2 and its other binding partners (e.g., bacteria, yeast, LPS, ApoA-I). In some embodiments, such compositions are useful for increasing (e.g., costimulating, enhancing, upregulating) the interaction between HIN-2 and its receptor or other binding partners. In some embodiments, such compositions are useful for decreasing (e.g., inhibiting, reducing, downregulating) the interaction between HIN-2 and its receptor or other binding partners.

According to the present invention, a “pharmaceutically acceptable carrier” includes pharmaceutically acceptable excipients and/or pharmaceutically acceptable delivery vehicles, which are suitable for use in administration of the composition to a suitable in vitro, ex vivo or in vivo site. A suitable in vitro, in vivo or ex vivo site is preferably any site where an innate immune system response is occurring or might occur, and/or where inflammation is occurring or may occur. In one embodiment, a suitable site is a lung tissue, a liver tissue, inflamed tissue, a site of a bacterial infection, a yeast infection, a site of a tumor, or a site where a monocyte or macrophage resides or is mobilized. Preferred pharmaceutically acceptable carriers are capable of maintaining a protein, compound, or recombinant nucleic acid molecule of the present invention in a form that, upon arrival of the protein, compound, or recombinant nucleic acid molecule at the cell target in a culture or in patient, the protein, compound or recombinant nucleic acid molecule is capable of interacting with its target (e.g., a naturally occurring HIN-2 protein, a Hin2 gene, a HIN-2 receptor, a gene encoding a HIN-2 receptor, a downstream gene or protein from HIN-2 receptor, or another ligand for HIN-2).

Suitable excipients of the present invention include excipients or formularies that transport or help transport, but do not specifically target a composition to a cell (also referred to herein as non-targeting carriers). Examples of pharmaceutically acceptable excipients include, but are not limited to water, phosphate buffered saline, Ringer's solution, dextrose solution, serum-containing solutions, Hank's solution, other aqueous physiologically balanced solutions, oils, esters and glycols. Aqueous carriers can contain suitable auxiliary substances required to approximate the physiological conditions of the recipient, for example, by enhancing chemical stability and isotonicity. Compositions of the present invention can be sterilized by conventional methods and/or lyophilized.

One type of pharmaceutically acceptable carrier includes a controlled release formulation that is capable of slowly releasing a composition of the present invention into a patient or culture. As used herein, a controlled release formulation comprises a compound of the present invention (e.g., a protein (including homologues), an antibody, a nucleic acid molecule, or a mimetic) in a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. Other carriers of the present invention include liquids that, upon administration to a patient, form a solid or a gel in situ. Preferred carriers are also biodegradable (i.e., bioerodible). When the compound is a recombinant nucleic acid molecule, suitable carriers include, but are not limited to liposomes, viral vectors or other carriers, including ribozymes, gold particles, poly-L-lysine/DNA-molecular conjugates, and artificial chromosomes. Natural lipid-containing carriers include cells and cellular membranes. Artificial lipid-containing carriers include liposomes and micelles.

A carrier of the present invention can be modified to target to a particular site in a patient, thereby targeting and making use of a compound of the present invention at that site. A pharmaceutically acceptable carrier which is capable of targeting can also be referred to herein as a “delivery vehicle” or “targeting carrier”. Suitable modifications include manipulating the chemical formula of the lipid portion of the delivery vehicle and/or introducing into the vehicle a targeting agent capable of specifically targeting a delivery vehicle to a preferred site or target site, for example, a preferred cell type. A “target site” refers to a site in a patient to which one desires to deliver a composition. Suitable targeting compounds include ligands capable of selectively (i.e., specifically) binding another molecule at a particular site. Examples of such ligands include antibodies, antigens, receptors and receptor ligands. Manipulating the chemical formula of the lipid portion of the delivery vehicle can modulate the extracellular or intracellular targeting of the delivery vehicle. For example, a chemical can be added to the lipid formula of a liposome that alters the charge of the lipid bilayer of the liposome so that the liposome fuses with particular cells having particular charge characteristics.

In one embodiment, HIN-2 can be used as a targeting molecule to deliver another protein or compound to a cell that expresses a HIN-2 receptor, such as MARCO. For example, HIN-2 can be fused or otherwise linked to a therapeutic protein, antigen or other desired compound and used to deliver such compound to a macrophage or tumor cell, for example.

One preferred delivery vehicle of the present invention is a liposome. A liposome is capable of remaining stable in an animal for a sufficient amount of time to deliver a nucleic acid molecule described in the present invention to a preferred site in the animal. A liposome, according to the present invention, comprises a lipid composition that is capable of delivering a nucleic acid molecule described in the present invention to a particular, or selected, site in a patient. A liposome according to the present invention comprises a lipid composition that is capable of fusing with the plasma membrane of the targeted cell to deliver a nucleic acid molecule into a cell. Suitable liposomes for use with the present invention include any liposome. Preferred liposomes of the present invention include those liposomes commonly used in, for example, gene delivery methods known to those of skill in the art. More preferred liposomes comprise liposomes having a polycationic lipid composition and/or liposomes having a cholesterol backbone conjugated to polyethylene glycol. Complexing a liposome with a nucleic acid molecule of the present invention can be achieved using methods standard in the art.

Another preferred delivery vehicle comprises a viral vector. A viral vector includes an isolated nucleic acid molecule useful in the present invention, in which the nucleic acid molecules are packaged in a viral coat that allows entrance of DNA into a cell. A number of viral vectors can be used, including, but not limited to, those based on alphaviruses, poxviruses, adenoviruses, herpesviruses, lentiviruses, adeno-associated viruses and retroviruses.

Another embodiment of the present invention relates to a method to regulate biological processes, including innate immune processes and inflammation, by regulating the activity of HIN-2 and/or its receptors. This embodiment can generally include the use (e.g., administration) of therapeutic compositions comprising one or more of HIN-2 proteins, nucleic acid molecules comprising nucleic acid sequence encoding HIN-2, antibodies that specifically bind to HIN-2, agonists or antagonists of HIN-2 proteins, HIN-2 receptor proteins, nucleic acid molecules comprising nucleic acid sequence encoding HIN-2 receptor, antibodies that specifically bind to HIN-2 receptor, and/or agonists or antagonists of HIN-2 receptors, that are useful in a method of regulating biological processes, including immune processes, that are mediated by or associated with the expression and biological activity of HIN-2 and its receptors.

Accordingly, one aspect of the invention relates to a method to regulate HIN-2 expression or activity, comprising administering to a patient a regulatory compound that regulates the biological activity of HIN-2. The regulatory compound can include any of the heretofore described regulators of HIN-2 expression or activity, including, but not limited to, HIN-2 itself or a biologically active fragment thereof, a HIN-2 homologue (including agonists or antagonists), HIN-2 or HIN-2 receptor binding agents (antibodies, antigen binding fragments, binding partners), HIN-2 receptors (e.g., MARCO) or homologues thereof (including soluble receptors, receptor agonists or receptor antagonists), synthetic and peptide mimetics, and/or nucleic acid molecules (encoding HIN-2 or a HIN-2 receptor, antisense, ribozymes).

In one aspect, the regulatory compound binds to a HIN-2 receptor (e.g., MARCO) or another ligand bound by HIN-2 (e.g., bacteria, yeast, LPS, ApoA-I). In one embodiment, the regulatory compound regulates phagocytosis of bacteria by a macrophage. In another embodiment, the regulatory compound regulates the phagocytosis of yeast by a host cell. In one embodiment, the regulatory compound binds to MARCO and induces NFκB activation in a cell expressing MARCO. In one embodiment, the regulatory compound the regulates the ability of HIN-2 to bind to a HIN-2 receptor or its other ligands. In one embodiment, the regulatory compound regulates the expression of HIN-2. In another embodiment, the regulatory compound regulates inflammation in a patient (e.g., Th1-type or Th-2/allergic inflammation). In another embodiment, the regulatory compound regulates the innate immune response in a patient. In another embodiment, the regulatory compound regulates the levels of high density lipoproteins or low density lipoproteins in a patient. In yet another embodiment, the regulatory compound ameliorates at least one symptom of cancer in a patient.

Accordingly, in one embodiment, the method of the present invention preferably regulates an immune response in a patient, and particularly an innate immune response and/or inflammation, such that the patient is protected from a disease that is amenable to regulation of an immune response, such as infectious disease, autoimmune disease, allergic inflammation, and cancer. As used herein, the phrase “protected from a disease” refers to reducing the symptoms of the disease; reducing the occurrence of the disease, and/or reducing the severity of the disease. Protecting a patient can refer to the ability of a therapeutic composition of the present invention, when administered to a patient, to prevent a disease from occurring and/or to cure or to treat the disease by alleviating disease symptoms, signs or causes. As such, to protect a patient from a disease includes both preventing disease occurrence (prophylactic treatment) and treating a patient that has a disease or that is experiencing initial symptoms or later stage symptoms of a disease (therapeutic treatment). In particular, protecting a patient from a disease can be accomplished by regulating an immune response in the patient by inducing a beneficial or protective immune response which may, in some instances, additionally suppress (e.g., reduce, inhibit or block) an overactive or harmful immune response (e.g., upregulation of proinflammatory cytokines or Th1 cytokines can act to suppress a harmful Th2 cytokine response, and vice versa). The term, “disease” refers to any deviation from the normal health of a patient and includes a state when disease symptoms are present, as well as conditions in which a deviation (e.g., infection, gene mutation, genetic defect, etc.) has occurred, but symptoms are not yet manifested (e.g., a precancerous condition).

More specifically, a therapeutic composition as described herein, when administered to a patient by the method of the present invention, preferably produces a result which can include alleviation of the disease (e.g., reduction of at least one symptom or clinical manifestation of the disease), elimination of the disease, reduction in inflammation associated with the disease, increased clearance of infectious organisms associated with the disease, reduction of a tumor or lesion associated with the disease, elimination of a tumor or lesion associated with the disease, prevention or alleviation of a secondary disease resulting from the occurrence of a primary disease, prevention of the disease, and initial control or induction of effector cell immunity and/or humoral immunity (i.e., adaptive immunity) against the disease.

According to the present invention, an effective administration protocol (i.e., administering a therapeutic composition in an effective manner) comprises suitable dose parameters and modes of administration that result in the desired effect in the patient (e.g., reduction of inflammation, enhancement or suppression of innate immun responses), preferably so that the patient is protected from the disease (e.g., by disease prevention or by alleviating one or more symptoms of ongoing disease). Effective dose parameters can be determined using methods standard in the art for a particular disease. Such methods include, for example, determination of survival rates, side effects (i.e., toxicity) and progression or regression of disease.

In accordance with the present invention, a suitable single dose size is a dose that results in regulation of inflammation and/or an aspect of the innate immune response or other immune response in a patient, or in the amelioration of at least one symptom of a condition in the patient, when administered one or more times over a suitable time period. Doses can vary depending upon the disease being treated. For example, in the treatment of cancer, a suitable single dose can be dependent upon whether the cancer being treated is a primary tumor or a metastatic form of cancer. One of skill in the art can readily determine appropriate single dose sizes for a given patient based on the size of a patient and the route of administration. One of skill in the art can monitor the effectiveness of the treatment by measuring, for example, up- or downregulation of immune activation markers on cells, cytokine responses, immediate hypersensitivity responses, production of non-cytokine inflammatory mediators, microbial load in a given cell or tissue, or other indicators of inflammation (e.g., swelling, vasodilation, airway hyperresponsiveness), and symptoms associated with a specific disease or condition.

In one aspect of the invention, a suitable single dose of a therapeutic composition of the present invention is an amount that, when administered by any route of administration, regulates at least one parameter of HIN-2 biological activity in the patient as described above, as compared to a patient which has not been administered with the therapeutic composition of the present invention (i.e., a control patient), as compared to the patient prior to administration of the vaccine or composition, or as compared to a standard established for the particular disease, patient type and composition.

A suitable single dose of a therapeutic composition to regulate a cancer or tumor is an amount that is sufficient to reduce, stop the growth of, and preferably eliminate, the tumor following administration of the composition into the tissue of the patient that has cancer. A single dose of a therapeutic composition useful to regulate inflammation and/or an aspect of an innate immune response in a patient with an infectious disease and/or against a lesion associated with such a disease is substantially similar to those doses used to treat a tumor, wherein the amount is sufficient to reduce, eliminate, or prevent at least one symptom of an infectious disease or lesion associated with such disease. Similarly, a single dose of a therapeutic composition useful to regulate inflammation and/or an aspect of an innate immune response in a patient with an allergic inflammatory condition is substantially similar to those doses used to treat a tumor, wherein the amount is sufficient to reduce, eliminate or prevent at least one symptom of allergic inflammation.

As discussed above, a therapeutic composition of the present invention is administered to a patient in a manner effective to deliver the composition to a cell, a tissue, and/or systemically to the patient, whereby the desired result (e.g., clearance of a bacterial infection, clearance of yeast infection, regulation of inflammation, regulation of other aspects of innate immunity) is achieved as a result of the administration of the composition. Suitable administration protocols include any in vivo or ex vivo administration protocol. The preferred routes of administration will be apparent to those of skill in the art, depending on the type of condition to be prevented or treated; whether the composition is nucleic acid based, protein based, or cell based; and/or the target cell/tissue. For proteins or nucleic acid molecules, preferred methods of in vivo administration include, but are not limited to, intravenous administration, intraperitoneal administration, intramuscular administration, intranodal administration, intracoronary administration, intraarterial administration (e.g., into a carotid artery), subcutaneous administration, transdermal delivery, intratracheal administration, subcutaneous administration, intraarticular administration, intraventricular administration, inhalation (e.g., aerosol), intracranial, intraspinal, intraocular, intranasal, oral, bronchial, rectal, topical, vaginal, urethral, pulmonary administration, impregnation of a catheter, and direct injection into a tissue. In one embodiment, routes of delivery which are useful for delivery to the lung tissue are preferred, In another embodiment, routes of delivery which are useful for delivery to an inflamed joint or other site of inflammation are preferred. Routes useful for deliver to mucosal tissues include, bronchial, intradermal, intramuscular, intranasal, other inhalatory, rectal, subcutaneous, topical, transdermal, vaginal and urethral routes. Combinations of routes of delivery can be used and in some instances, may enhance the therapeutic effects of the composition.

Ex vivo administration refers to performing part of the regulatory step outside of the patient, such as administering a composition (nucleic acid or protein) of the present invention to a population of cells removed from a patient under conditions such that the composition contacts and/or enters the cell, and returning the cells to the patient. Ex vivo methods are particularly suitable when the target cell type can easily be removed from and returned to the patient.

Many of the above-described routes of administration, including intravenous, intraperitoneal, intradermal, and intramuscular administrations can be performed using methods standard in the art. Aerosol (inhalation) delivery can also be performed using methods standard in the art (see, for example, Stribling et al., Proc. Natl. Acad. Sci. USA 189:11277-11281, 1992, which is incorporated herein by reference in its entirety). Oral delivery can be performed by complexing a therapeutic composition of the present invention to a carrier capable of withstanding degradation by digestive enzymes in the gut of an animal. Examples of such carriers, include plastic capsules or tablets, such as those known in the art.

One method of local administration is by direct injection. Direct injection techniques are particularly useful for administering a composition to a cell or tissue that is accessible by surgery, and particularly, on or near the surface of the body. Administration of a composition locally within the area of a target cell refers to injecting the composition centimeters and preferably, millimeters from the target cell or tissue.

Various methods of administration and delivery vehicles disclosed herein have been shown to be effective for delivery of a nucleic acid molecule to a target cell, whereby the nucleic acid molecule transfected the cell and was expressed. In many studies, successful delivery and expression of a heterologous gene was achieved in preferred cell types and/or using preferred delivery vehicles and routes of administration of the present invention. All of the publications discussed below and elsewhere herein with regard to gene delivery and delivery vehicles are incorporated herein by reference in their entirety.

For example, using liposome delivery, U.S. Pat. No.5,705,151, issued Jan. 6, 1998, to Dow et al. demonstrated the successful in vivo intravenous delivery of a nucleic acid molecule encoding a superantigen and a nucleic acid molecule encoding a cytokine in a cationic liposome delivery vehicle, whereby the encoded proteins were expressed in tissues of the animal, and particularly in pulmonary tissues. In addition, Liu et al., Nature Biotechnology 15:167, 1997, demonstrated that intravenous delivery of cholesterol-containing cationic liposomes containing genes preferentially targets pulmonary tissues and effectively mediates transfer and expression of the genes in vivo. Several publications by Dzau and collaborators demonstrate the successful in vivo delivery and expression of a gene into cells of the heart, including cardiac myocytes and fibroblasts and vascular smooth muscle cells using both naked DNA and Hemagglutinating virus of Japan-liposome delivery, administered by both incubation within the pericardium and infusion into a coronary artery (intracoronary delivery) (See, for example, Aoki et al., 1997, J. Mol. Cell, Cardiol. 29:949-959; Kaneda et al., 1997, Ann N.Y. Acad Sci. 811:299-308; and von der Leyen et al., 1995, Proc Natl Acad Sci USA 92:1137-1141).

Delivery of numerous nucleic acid sequences has been accomplished by administration of viral vectors encoding the nucleic acid sequences. Using such vectors, successful delivery and expression has been achieved using ex vivo delivery (See, of many examples, retroviral vector; Blaese et al., 1995, Science 270:475-480; Bordignon et al., 1995, Science 270:470-475), nasal administration (CFTR-adenovirus-associated vector), intracoronary administration (adenoviral vector and Hemagglutinating virus of Japan, see above), intravenous administration (adeno-associated viral vector; Koeberl et al., 1997, Proc Natl Acad Sci USA 94:1426-1431). A publication by Maurice et al. (1999, J. Clin. Invest. 104:21-29) demonstrated that an adenoviral vector encoding a β2-adrenergic receptor, administered by intracoronary delivery, resulted in diffuse multichamber myocardial expression of the gene in vivo, and subsequent significant increases in hemodynamic function and other improved physiological parameters. Levine et al. describe in vitro, ex vivo and in vivo delivery and expression of a gene to human adipocytes and rabbit adipocytes using an adenoviral vector and direct injection of the constructs into adipose tissue (Levine et al., 1998, J. Nutr. Sci. Vitaminol. 44:569-572).

In the area of neuronal gene delivery, multiple successful in vivo gene transfers have been reported. Millecamps et al. reported the targeting of adenoviral vectors to neurons using neuron restrictive enhancer elements placed upstream of the promoter for the transgene (phosphoglycerate promoter). Such vectors were administered to mice and rats intramuscularly and intracerebrally, respectively, resulting in successful neuronal-specific transfection and expression of the transgene in vivo (Millecamps et al., 1999, Nat. Biotechnol. 17:865-869). As discussed above, Bennett et al. reported the use of adeno-associated viral vector to deliver and express a gene by subretinal injection in the neural retina in vivo for greater than 1 year (Bennett, 1999, ibid.).

Gene delivery to synovial lining cells and articular joints has had similar successes. Oligino and colleagues report the use of a herpes simplex viral vector which is deficient for the immediate early genes, ICP4, 22 and 27, to deliver and express two different receptors in synovial lining cells in vivo (Oligino et al., 1999, Gene Ther. 6:1713-1720). The herpes vectors were administered by intraarticular injection. Kuboki et al. used adenoviral vector-mediated gene transfer and intraarticular injection to successfully and specifically express a gene in the temporomandibular joints of guinea pigs in vivo (Kuboki et al., 1999, Arch. Oral. Biol. 44:701-709). Apparailly and colleagues systemically administered adenoviral vectors encoding IL-10 to mice and demonstrated successful expression of the gene product and profound therapeutic effects in the treatment of experimentally induced arthritis (Apparailly et al., 1998, J. Immunol. 160:5213-5220). In another study, murine leukemia virus-based retroviral vector was used to deliver (by intraarticular injection) and express a human growth hormone gene both ex vivo and in vivo (Ghivizzani et al., 1997, Gene Ther. 4:977-982). This study showed that expression by in vivo gene transfer was at least equivalent to that of the ex vivo gene transfer. As discussed above, Sawchuk et al. has reported successful in vivo adenoviral vector delivery of a gene by intraarticular injection, and prolonged expression of the gene in the synovium by pretreatment of the joint with anti-T cell receptor monoclonal antibody (Sawchuk et al., 1996, ibid. Finally, it is noted that ex vivo gene transfer of human interleukin-1 receptor antagonist using a retrovirus has produced high level intraarticular expression and therapeutic efficacy in treatment of arthritis, and is now entering FDA approved human gene therapy trials (Evans and Robbins, 1996, Curr. Opin. Rheumatol. 8:230-234). Therefore, the state of the art in gene therapy has led the FDA to consider human gene therapy an appropriate strategy for the treatment of at least arthritis. Taken together, all of the above studies in gene therapy indicate that delivery and expression of a recombinant nucleic acid molecule according to the present invention is feasible.

Another method of delivery of recombinant molecules is in a non-targeting carrier (e.g., as “naked” DNA molecules, such as is taught, for example in Wolff et al., 1990, Science 247, 1465-1468). Such recombinant nucleic acid molecules are typically injected by direct or intramuscular administration. Recombinant nucleic acid molecules to be administered by naked DNA administration include an isolated nucleic acid molecule of the present invention, and preferably includes a recombinant molecule of the present invention that preferably is replication, or otherwise amplification, competent. A naked nucleic acid reagent of the present invention can comprise one or more nucleic acid molecules of the present invention including a dicistronic recombinant molecule. Naked nucleic acid delivery can include intramuscular, subcutaneous, intradermal, transdermal, intranasal and oral routes of administration, with direct injection into the target tissue being most preferred. A preferred single dose of a naked nucleic acid vaccine ranges from about 1 nanogram (ng) to about 100 μg, depending on the route of administration and/or method of delivery, as can be determined by those skilled in the art. Suitable delivery methods include, for example, by injection, as drops, aerosolized and/or topically. In one embodiment, pure DNA constructs cover the surface of gold particles (1 to 3 μm in diameter) and are propelled into skin cells or muscle with a “gene gun.”

In the method of the present invention, vaccines and therapeutic compositions can be administered to any member of the Vertebrate class, Mammalia, including, without limitation, primates, rodents, livestock and domestic pets. Livestock include mammals to be consumed or that produce useful products (e.g., sheep for wool production). Preferred mammals to protect include humans, dogs, cats, mice, rats, sheep, cattle, horses and pigs, with humans and dogs being particularly preferred, and humans being most preferred.

Conditions to treat using methods of the present invention include any condition, disease in which it is useful to modulate the activity of HIN-2 or its receptor(s). Such conditions include, but are not limited to, any condition in which the innate immune response plays a role, microbial infections (especially bacterial or yeast, any condition in which inflammation occurs (Th1 or Th2-type), and cancer. In one embodiment, such a condition is a lung condition. Lung conditions include, but are not limited to, asthma, allergic bronchopulmonary aspergillosis, hypersensitivity pneumonia, eosinophilic pneumonia, emphysema, bronchitis, allergic bronchitis bronchiectasis, cystic fibrosis, tuberculosis, hypersensitivity pneumotitis, occupational asthma (i.e., asthma, wheezing, chest tightness and cough caused by a sensitizing agent, such as an allergen, irritant or hapten, in the work place), sarcoid, reactive airway disease syndrome (i.e., a single exposure to an agent that leads to asthma), interstitial lung disease, hyper-eosinophilic syndrome, rhinitis, sinusitis, or parasitic lung disease. Particularly preferred conditions to treat are asthma, interstitial lung disease, and cystic fibrosis, and any disease of the lung where inflammation and/or microbial infection is involved. In one embodiment, the condition is an arthritic disease, such as reactive arthritis, rheumatoid arthritis or spondylarthropathy. In one embodiment, the condition is an autoimmune disease, including, but not limited to, rheumatoid arthritis, systemic lupus erythematosus, insulin dependent diabetes mellitis, multiple sclerosis, myasthenia gravis, Grave's disease, autoimmune hemolytic anemia, autoimmune thrombocytopenia purpura, Goodpasture's syndrome, pemphigus vulgaris, acute rheumatic fever, post-streptococcal glomerulonephritis, or polyarteritis nodosa. In another embodiment, the disease is any bacterial infectious disease such as those caused by Cryptococcus, Bacillus anthracis and Yersenia pestis, Staphylococcus, Pseudomonas, Streptococcus, Enterococcus, Salmonella, Pasteurella, Fransicella and other gram negative and gram positive bacterial pathogens, as well as diseases caused by mycobacteria (e.g., M. tuberculosis). In another embodiment, the disease is any yeast infectious disease, such as those caused by pathogenic yeast. Diseases caused by other microbial infections, including viruses, parasites (including internal parasites), other fungi (including pathogenic fungi), endoparasites, ectoparasites, and prions (e.g., bovine spongiform encephalopathy; BSE) may also be treated using methods of the present invention. In another embodiment, the disease to be treated can include a cancer, especially a cancer wherein tumor cells express a HIN-2 receptor (e.g., some lung tumor cells express MARCO). Cancers include, but are not limited to, melanomas, squamous cell carcinoma, breast cancers, head and neck carcinomas, thyroid carcinomas, soft tissue sarcomas, bone sarcomas, testicular cancers, prostatic cancers, ovarian cancers, bladder cancers, skin cancers, brain cancers, angiosarcomas, hemangiosarcomas, mast cell tumors, primary hepatic cancers, lung cancers, pancreatic cancers, gastrointestinal cancers, renal cell carcinomas, hematopoietic neoplasias and metastatic cancers thereof. In yet another embodiment, the disease or condition that can be treated by modulation of the level of high density lipoproteins or low density lipoproteins in the patient (e.g., atherosclerosis).

Yet another embodiment of the invention relates to a genetically modified non-human animal comprising a genetic modification within at least one allele of its Hin-2 locus, wherein the genetic modification results in a reduction of HIN-2 biological activity in the animal. In one embodiment, the animal comprises a genetic modification in both alleles of its Hin-2 locus, wherein the genetic modification results in an absence of HIN-2 biological activity in the animal.

According to the present invention, a “genetically modified” animal has a genome which is modified (i.e., mutated or changed) from its normal (i.e., wild-type or naturally occurring) form such that the desired result is achieved (e.g., a reduction in the action of HIN-2). Genetic modification of an animal is typically accomplished using molecular genetic and cellular techniques, including manipulation of embryonic cells and DNA (e.g., DNA comprising the Hin-2 locus). Such techniques are generally disclosed for mice, for example, in “Manipulating the Mouse Embryo” (Hogan et al., Cold Spring Harbor Laboratory Press, 1994, incorporated herein by reference in its entirety).

A genetically modified non-human animal can include a non-human animal in which nucleic acid molecules have been modified (i.e., mutated; e.g., by insertion, deletion, substitution, and/or inversion of nucleotides), in such a manner that such modifications provide the desired effect within the animal (i.e., reduction in HIN-2 peptide action). As used herein, genetic modifications which result in a reduction in gene expression, in the function of the gene, or in the function of the gene product (i.e., the protein encoded by the gene) can be referred to as inactivation (complete or partial), deletion, interruption, blockage or down-regulation of a gene. For example, a genetic modification in a gene which results in a decrease in the function of the protein encoded by such gene, can be the result of: a partial or complete deletion of the gene or of an exon within the gene (i.e., the gene does not exist, and therefore the protein can not be produced); a mutation (e.g., a deletion, substitution, insertion and/or inversion) in the gene which results in incomplete or no translation of the protein (e.g., a mutation which causes a frame shift so that the correct protein is not expressed, a mutation in one or more exons of the gene so that the protein or at least a portion of the protein is not expressed, or a mutation in a regulatory region so that the protein is not expressed or has reduced expression); or a mutation in the gene which decreases or abolishes the natural function of the protein (e.g., a protein is expressed which has decreased or no biological activity or action).

As used herein, a non-human animal suitable for genetic modification according to the present invention is any non-human animal for which the Hin-2 locus can be manipulated, including non-human members of the Vertebrate class, Mammalia, such as non-human primates and rodents. Preferably, such a non-human animal is a rodent, and more preferably, a mouse.

Techniques for achieving targeted integration of an isolated nucleic acid molecule into a genome are well known in the art and are described, for example in “Manipulating the Mouse Embryo”, supra. For example, the isolated nucleic acid molecule can be engineered into a targeting vector which is designed to integrate into a host genome. According to the present invention, a targeting vector is defined as a nucleic acid molecule which has the following three features: (1) genomic sequence from the target locus in the host genome to stimulate homologous recombination at that locus; (2) a desired genetic modification within the genomic sequence from the target locus sufficient to obtain the desired phenotype; and (3) a selectable marker (e.g., an antibiotic resistance cassette, such as G418, neomycin, or hygromycin resistance cassettes). Such targeting vectors are well known in the art. Following introduction of the isolated nucleic acid molecule of the targeting vector into the ES cells, ES cells which homologously integrate the isolated nucleic acid molecule are injected into mouse blastocysts and chimeric mice are produced. These mice are then bred onto the desired mouse background to detect those which transmit the mutated gene through the germ line. Heterozygous offspring of germline transmitting lines can then be mated to produce homozygous progeny.

Non-human animals which carry one or more mutated Hin-2 alleles can be identified using any suitable method for evaluating DNA. For example, genotypes can be analyzed by PCR and confirmed by Southern blot analysis as described (Sambrook et al., 1988, Molecular Cloning: A Laboratory Manual, Cold Spring Harbor Press, Cold Spring Harbor Laboratory, Cold Spring Harbor, N.Y. or Current Protocols in Molecular Biology (1989) and supplements).

Another embodiment includes a method to detect a polymorphism or a loss of heterozygosity located in chromosome region 5q30-40, comprising incubation of a sample with an oligonucleotide comprising at least 8 nucleotides of an isolated Hin-2 polynucleotide, homologue thereof, or a nucleic acid sequence fully complementary thereto. The effect of a polymorphism in Hin-2 gene sequence on the particular phenotype is determined by in vitro or in vivo assays. Generally, in vitro assays are useful in determining the direct effect of a particular polymorphism, while clinical studies will also detect a phenotype that is genetically linked to a polymorphism.

A number of methods are available for analyzing nucleic acids for the presence of a specific sequence. Where large amounts of DNA are available, genomic DNA is used directly. Alternatively, the region of interest is cloned into a suitable vector and grown in sufficient quantity for analysis. The nucleic acid may be amplified by conventional techniques, such as the polymerase chain reaction (PCR), to provide sufficient amounts for analysis. The use of the polymerase chain reaction is described in Saiki et al. (1985) Science 239:487, and a review of current techniques may be found in Sambrook et al. Molecular Cloning: A Laboratory Manual, CSH Press 1989, pp.14.2-14.33. Amplification may be used to determine whether a polymorphism is present, by using a primer that is specific for the polymorphism. Alternatively, various methods are known in the art that utilize oligonucleotide ligation as a means of detecting polymorphisms, for examples see Riley et al. (1990) N.A.R. 18:2887-2890; and Delahunty et al. (1996) Am. J. Hum. Genet.58:1239-1246.

A detectable label may be included in an amplification reaction. Suitable labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g. 32P, 35S, 3H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

The sample nucleic acid, e.g. amplified or cloned fragment, is analyzed by one of a number of methods known in the art. The nucleic acid may be sequenced by dideoxy or other methods. Hybridization with the variant sequence may also be used to determine its presence, by Southern blots, dot blots, etc. The hybridization pattern of a control and variant sequence to an array of oligonucleotide probes immobilized on a solid support, as described in U.S. Pat. No. 5,445,934, or in PCT Publication No. WO 95/35505 (each incorporated herein by reference in its entirety), may also be used as a means of detecting the presence of variant sequences. Single strand conformational polymorphism (SSCP) analysis, denaturing gradient gel electrophoresis (DGGE), mismatch cleavage detection, and heteroduplex analysis in gel matrices are used to detect conformational changes created by DNA sequence variation as alterations in electrophoretic mobility. Alternatively, where a polymorphism creates or destroys a recognition site for a restriction endonuclease (restriction fragment length polymorphism, RFLP), the sample is digested with that endonuclease, and the products size fractionated to determine whether the fragment was digested. Fractionation is performed by gel or capillary electrophoresis, particularly acrylamide or agarose gels.

Genotyping is performed by DNA or RNA sequence and/or hybridization analysis of any convenient sample from a patient, e.g. biopsy material, tissue sample, blood sample, scrapings from cheek, etc. A nucleic acid sample from an individual is analyzed for the presence of polymorphisms in Hin-2, particularly those that affect the activity or expression of Hin-2. Specific sequences of interest include any polymorphism that leads to changes in basal expression in one or more tissues, to changes in the modulation of Hin-2 expression by or alterations in Hin-2 activity.

Diagnostic screening may also be performed for polymorphisms that are genetically linked to a phenotypic variant in HIN-2 activity or expression, particularly through the use of microsatellite markers or single nucleotide polymorphisms (SNP). The microsatellite or SNP polymorphism itself may not phenotypically expressed, but is linked to sequences that result in altered activity or expression. Two polymorphic variants may be in linkage disequilibrium, i.e. where alleles show non-random associations between genes even though individual loci are in Hardy-Weinberg equilibrium. Linkage analysis may be performed alone, or in combination with direct detection of phenotypically evident polymorphisms. The use of microsatellite markers for genotyping is well documented. For examples, see Mansfield et al. (1994) Genomics 24:225-233; and Ziegle et al. (1992) Genomics 14:1026-1031. The use of SNPs for genotyping is illustrated in Golevleva et al. (1996) Am. J. Hum. Genet. 59:570-578; and in Underhill et al. (1996) P.N.A.S. 93:196-200; all incorporated by reference in their entirety.

The following examples are provided for the purpose of illustration and are not intended to limit the scope of the present invention.

### EXAMPLES

### Example 1

This example describes the cloning, expression and initial characterization of HIN-2.

The human HIN-2 sequence (SEQ ID NO:1) was cloned into a PRK-C-flag plasmid and transfected into a cell line called 293 GT. Specifically, to construct the mammalian expression plasmid for FLAG-HIN2, a full length cDNA fragment encoding amino acids 1-94 of human HIN2 (SEQ ID NO:2) was amplified from an EST clone by PCR. The PCR product was digested with EcoR1 and Xba I, and inserted into the EcoRI and Xba I sites of PRK-c-FLAG plasmid to make PRK-C-FLAG-HIN2. To purify FLAG-HIN2,1500 ml of conditioned medium from the 293 cell expressing FLAG-HIN2 was collected and supplemented with 10 mM Tris (pH 7.5) and 150 mM NaCl. 2 ml of anti-flag antibody affinity chromatography column (Sigma, St. Luis, Mo.) was pre-washed with three sequential 5 ml aliquots of 0.1 M glycine (pH 3.5) and followed by three sequential washes with 5 ml of TBS buffer (50 mM Tris, 150 mM NaCl, pH 7.4). The medium was passed through the column and the column was washed with 12 ml aliquots of TBS three times. The protein that was bound to the column was eluted with 100 μg/ml FLAG peptide (Sigma, St. Luis, Mo.).

To detect FLAG-HIN-2 expression, 293 cells (3×10⁶/100 mm dish) were transfected with 10 μg of PRK-C-FLAG-HIN-2 or the empty control vector by Ca³(PO₃)₂ precipitation. 24 hours after transfection, cell culture medium was collected and lyophilized. 500 μl of 1×SDS was used to dissolved the lyophilized powders and 15% SDS-PAGE and western blot analysis was performed with anti-flag antibody. To sequence the purified protein, 2 μg of FLAG-HIN-2 were fractionated on SDS-PAGE, and then electroblotted onto ProBlott™ (PDVF) in 1×CAPS (10 mM CAPS, pH 11, 10% MeOH), 50 v, 3-4 hours. The ProBlot™ was removed from the transblotting sandwich and stained with 0.1% Coomassie® Blue R-250 in 40% MeOH/1% acetic acid for 1 min. The ProBlott™ was removed from the staining solution and destained with 50% MeOH. The blot was then rinsed extensively with D.I. water and bands of interest were excised for sequencing using standard techniques.

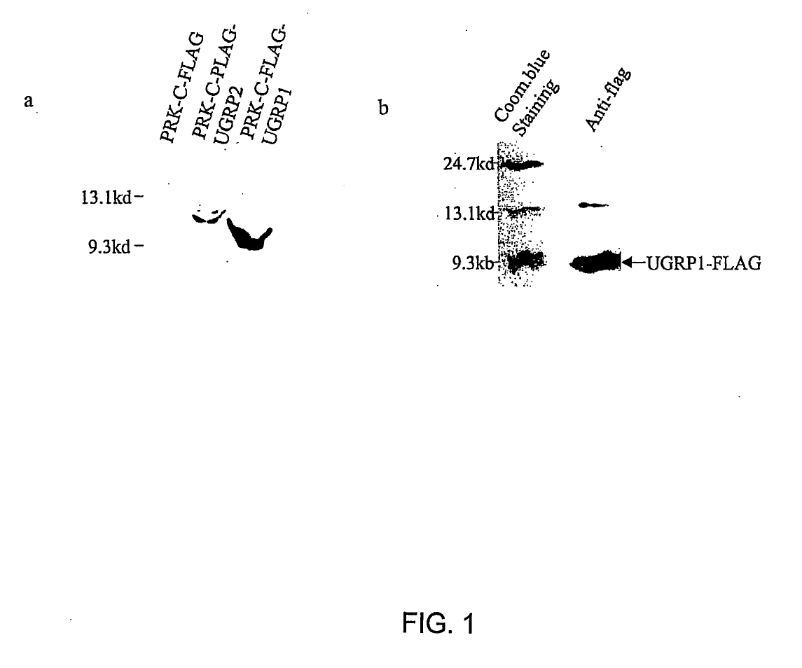

The sequencing revealed that the HIN-2 protein consisted of 93 amino acids. However, subsequent expression of the protein revealed that at the amino-terminal end there is a 21 amino acid signal peptide that is cleaved to release the remaining 72-amino acid sequence (amino acids 22-93 of SEQ ID NO:2) as a mature HIN-2 protein (86 amino acids with the Flag tag on the carboxy-terminus). The 293 GT cell line expressed the entire 93-AA sequence (SEQ ID NO:2) for the HIN-2 protein and when protein production was induced, the mature, 72 amino acid HIN-2 protein (plus the Flag tag sequence) was secreted into the medium. Expression of FLAG-tagged HIN-2 was confirmed by Western blot analysis using an anti-FLAG antibody, which showed the mature protein to be an about 10 kDa secreted protein (see FIG. 1A). The secreted FLAG-HIN2 protein was also purified using an anti-FLAG antibody affinity column (FIG. 1B). A protein sequence assay confirmed that the secreted protein was the mature protein, in which the N-terminal signal sequence had been spliced out (data not shown). This experiment also revealed that HIN-2 copurified with a protein that was identified as apolipoprotein A-I (ApoA-I), indicating that HIN-2 binds to ApoA-I (data not shown). These data indicate that the HIN-2 protein is a cytokine.

### Example 2

This example demonstrates the tissue distribution of HIN-2.

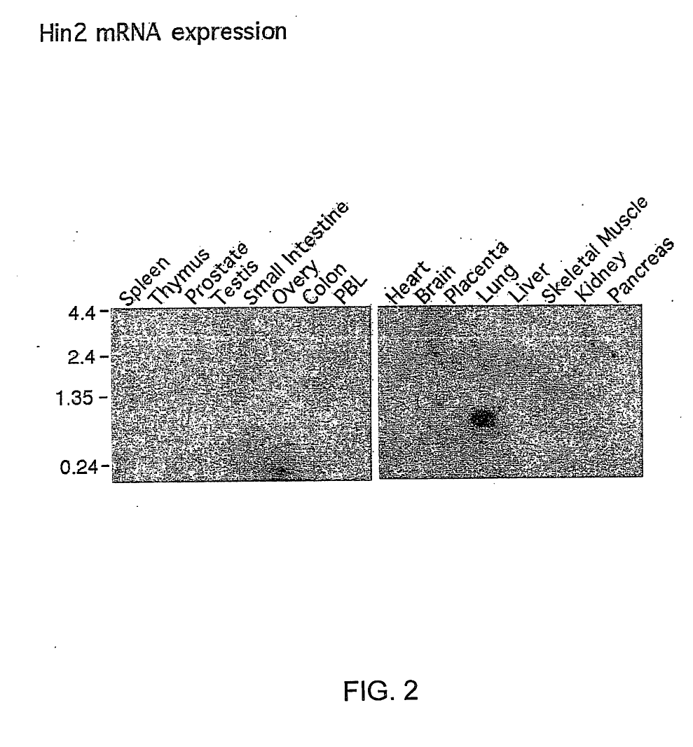

To determine the tissue distribution of Hin-2 expression, a Northern blot was performed. Briefly, a multiple tissue blotting kit was purchased from Clontech and full length Hin-2 cDNA was hybridized with the blots under stringent conditions. FIG. 2 is a Northern blot showing Hin-2 mRNA expression in a variety of tissue types. These results show that HIN-2 is expressed primarily in the lung.

### Example 3

This example demonstrates that HIN-2 binds to a receptor on lung cancer cell lines.

Since the Northern blot in Example 2 showed that HIN-2 is primarily expressed in the lung, the present inventors investigated several human lung cancer cell lines to assess the presence of a HIN-2 receptor and analyze binding of HIN-2 to such cells. For most experiments, cells (1×10⁶) were incubated with 100 ng FLAG-HIN-2 in staining buffer (D-PBS containing 2% fetal bovine serum) for 20 minutes, then sequentially incubated with anti-flag monoclonal antibody ( 1 μg/ml) and RPE-conjugated goat anti-mouse IgG (1:200 dilution), for 30 minutes each. Cells were washed twice with staining buffer following each incubation. The fluorescence exhibited by the stained cells was measured using a Becton Dickson FACScan flow cytometer.

Fluorescent cytometry results showed that the HIN-2 protein (FLAG-HIN-2) binds to a receptor on at least six different lung cancer cell lines, as detected by a shifting of the cell complex (data not shown).

### Example 4

This example demonstrates that MARCO is a receptor for HIN-2.

To identify a receptor for HIN-2, the inventors employed an expression cloning approach using U937 cells infected with a human lung retroviral library. Briefly, a human lung retroviral library was purchased from Strategene containing 1.8×10⁸ independent cDNA inserts. The library was transfected into the packaging cell line 293-10A1 using Ca₃(PO₄)₂ precipitation. Infectious retroviruses were harvested with 100 ml virus stock in the presence of polybrene (Sigma) (8 μg/ml). After two cycles of infection, FLAG-HIN-2 positive cells were enriched by Panning (FASC Inc.), which is briefly described below. Infected U937 cells were incubated with FLAG-HIN-2 (1 μg/ml) in staining buffer, then panned with magnetic beads conjugated to anti-mouse IgG in discarded buffer. The beads were washed twice with discarded buffer, and the positive cells were collected and grown in RPMI 1640 containing 10% FCS. After two rounds of enrichment for FLAG-HIN-2 positive cells, total RNA from these cells was prepared using Trizol (GiBco). To recover the integrated cDNA from the clones, reverse transcription was performed by using ScriptRTII (Invitrogen), and cDNA inserts were amplified by PCR with the pFB Vector primers (sense) antisense. The recovered clones showed as two separate bands on a gel (2.1 kb and 1.2 kb). The amplified cDNA fragments were subcloned into the TA vector (Promega) and sequenced. The clone corresponding to the 2.1 kb band was identified as encoding MARCO, a known scavenger receptor. The clone corresponding to the 1.2 kb band was identified as encoding SP-C, another receptor.

cDNA for both MARCO and SP-C were subcloned into the MSCV-IRES retroviral vector, which contains an IRES element followed by the gene encoding green fluorescent protein (GFP). Briefly, for MARCO, full length cDNA fragment encoding MARCO was amplified by PCR from the single MARCO expression cell clone cDNA with two primers from the human lung retroviral cDNA library (Strategene). The amplified cDNA fragment was digested with Sal I and Not I and cloned into the Xho I and Not I site of MSCV-GFP plasmid and PRK plasmids to make MSCV-MARCO and PRK-MARCO. SP-C cDNA was similarly prepared. Viruses were produced and used to infect U937 cells, and the infected cells were stained with FLAG-HIN-2. The FLAG-HIN-2 specifically stained the U937 cell line transduced with retroviruses encoding the MARCO, but no staining was detected on uninfected, MSCV-empty vector infected, or SP-C infected cells (data not shown). These data confirm that MARCO is a receptor for HIN-2, but SP-C appears to be a false positive clone.

### Example 5

This example demonstrates the tissue distribution of MARCO.

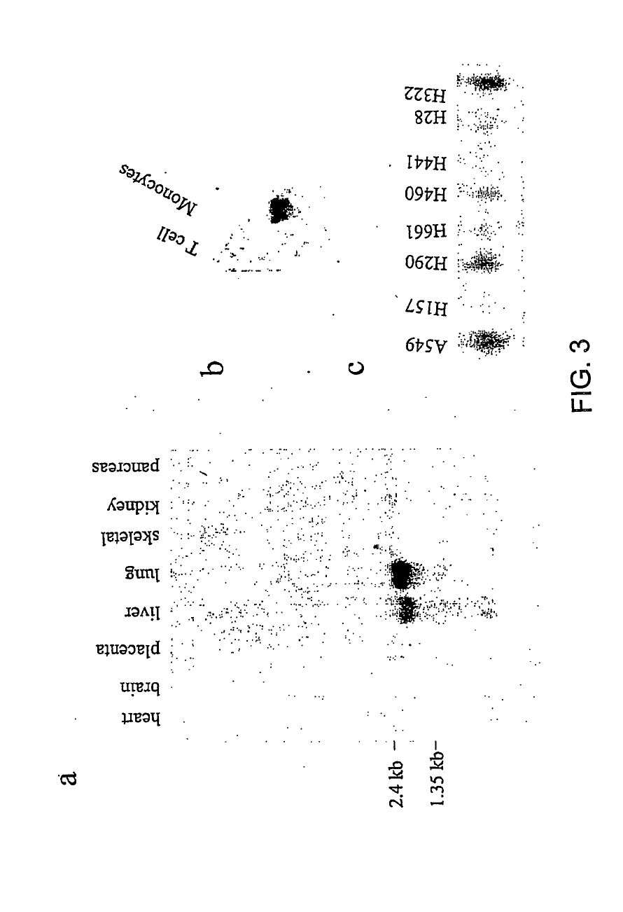

MARCO (macrophage receptor with a collagenous structure), belongs to the class A macrophage scavenger receptors, and can bind to bacteria and LPS. Prior studies have reported that murine MARCO is not found in the macrophages of lung or liver under normal conditions, but that human MARCO is strongly expressed in human lung, liver and peripheral monocytes. The present inventors performed Northern blots to detect the tissue distribution of MARCO. Briefly, total RNA from lung cancer cell lines, peripheral blood T cells and monocytes was prepared using TriZOL, was fractionated in 1.2% agarose gels, and then transferred to pure nitrocellulose membranes. Multiple tissue blotting membranes were purchased from Clontech. ³²P-labeled full-length cDNA encoding human MARCO was hybridized with blots under stringent conditions using standard techniques. The results demonstrated that human MARCO is strongly expressed in human lung, liver and peripheral monocytes, confirming studies by previous groups (See Northern blot, FIGS. 3A and 3B). Interestingly, the present inventors also found that MARCO was expressed in several lung cancer cell lines (FIG. 3C).

### Example 6

This example demonstrates that HIN-2 can bind to bacteria, yeast, and lipopolysaccharide (LPS).

The identification of MARCO as a receptor of HIN-2 provided the inventors with some information regarding HIN-2 function. The most important known function of MACRO is that it can bind to bacteria is then involved in the clearance of the microorganism via phagocytosis. Therefore, the present inventors, without being bound by theory, believe that HIN-2 may be involved in host defense. For example, HIN-2 may serve as chemokine that attracts macrophages to migrate to the location where the pathogen is attached. HIN-2 might also serve as a human antibacterial peptide, similar to β-defensin, etc., which is produced by airway epithelial cells and that can kill bacteria. Alternatively, BIN-2 might bind to bacteria and immobilize them, for example, to prevent the spread of infection.

To begin to test these hypotheses, the present inventors first used two lung infected bacteria strains, Listeria monocytogenes NP10 (gram positive) and Pseudomonas aeruginosa 01 (gram negative). HIN-2 protein was incubated with both alive bacteria and heat-killed bacteria from these two strains. Briefly, the Gram positive bacteria NP10 and Gram negative bacteria PA01 were grown in LB medium at the log-growth phase. Yeast were also grown for testing in this experiment. 500 μl bacteria (OD 600:0.5) were used for each sample. 0.2 μg HIN-2-FLAG was incubated with alive bacteria and heated-killed bacteria (80° C., 15 min) in 1×PBS (1 mg/ml BSA) or yeast and heat-killed yeast. The complexes were lysed in 20 μl 1×SDS buffer, loaded on 15% SDS-PAGE, and transferred to nitrocellulose membranes. Western blots were performed using anti-FLAG monoclonal antibody.

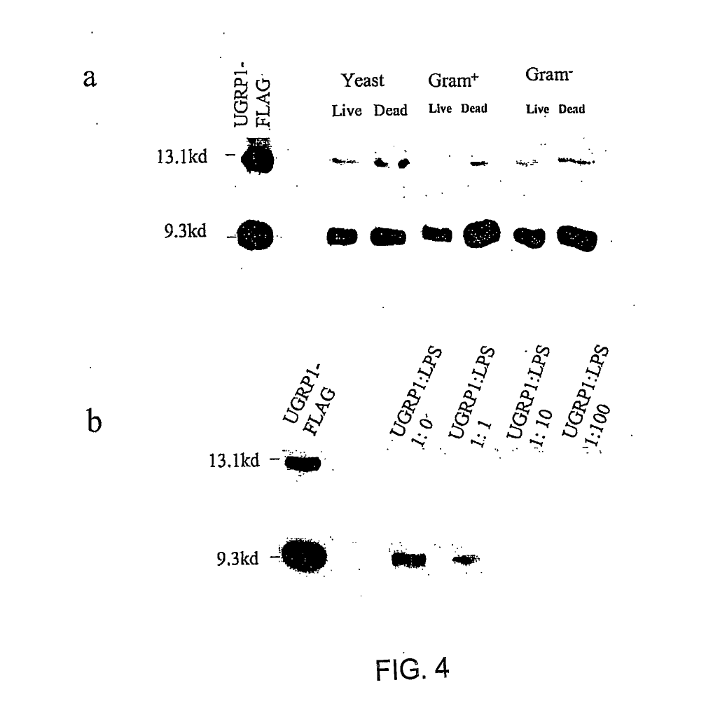

Surprisingly, the inventors found that HIN-2 can bind to both gram positive and gram negative bacteria, and both viable and non-viable bacteria, as well as to both viable and non-viable yeast (FIG. 4A). The control protein, FLAG-TALL-1 did not bind the bacteria or yeast (data not shown).

In another experiment, the inventors mixed LPS and HIN-2 in 1×PBS (including 1 mg/ml BSA) prior to incubating the composition with bacteria. Results showed that LPS inhibited the binding of HIN-2 to bacteria in a dose-dependent manner, which indicated that LPS also binds to HIN-2 (FIG. 4B). Therefore, the inventors have found that HIN-2 is capable of binding to both LPS and MARCO. In these experiments, neither chemotactic activity or anti-microbial activity by HIN-2 were detected.

In yet another experiment, flow cytometry showed that HIN-2 binds to MARCO-transfected U937 cells (the untransfected cell is negative for MARCO). Addition of LPS to the culture inhibited HIN-2 binding to the MARCO transfected U937 cells, indicating that LPS is a competitive inhibitor of HIN-2 for binding to MARCO.

### Example 7

This example shows that MARCO Activates NF-κB signaling pathway.

There is no report known to the inventors which describes the signal transduction pathway in MARCO when it is triggered by its ligand(s). MARCO has the same spectrum of known ligands (e.g., bacteria, LPS) with Toll-like receptor (TLR) family members, which play a very important role in host defense and innate immunity (e.g., see Medzhitov, 2001, Nature Rev. 1:135-145). The TLR family members will trigger the inflammatory response following NF-κB and AP1 activation. The TLR-activated signal transduction pathway proceeds from MyD88 to IRAK1 to TRAF6 to NF-κB/AP1. To determine whether MARCO has the same signal transduction pathway, the present inventors transfected PRK-MARCO into 293 GT cells with NF-κB luciferase reporter and AP1 luciferase reporter.

Briefly, each transfection was performed in triplicate in one experiment, and where necessary, enough of empty control plasmid was added to ensure that each transfection received the same amount of total DNA. To normalize for transfection efficiency and protein amount, 0.2 μg of RSV-B-GAL plasmid was added to all transfections. Luciferase activities were normalized on the basis of β-galactosidase expression levels. Data are averages and standard deviations of one representative experiment in which each transfection has been performed in triplicate.

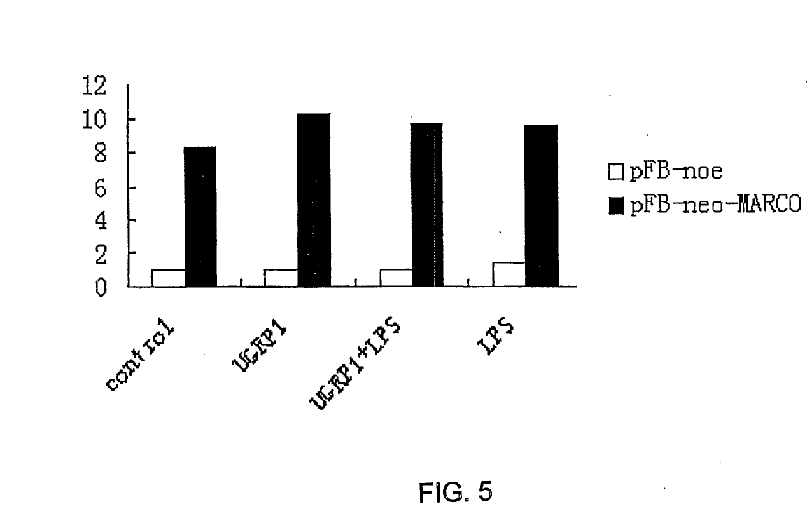

The results showed that over-expression of MARCO activated NF-κB (FIG. 5). This activation was blocked by dominate-negative TRAF6 (data not shown). This result suggested that MARCO shares the same signaling pathway with the TLR family. It is known that LPS and CD14 can potentiate the NF-κB activation of TLRs. However, the present inventors did not find the same phenomenon to exist in MARCO NF-κB signaling. Also, binding of HIN-2 to MARCO did not appear to activate NF-κB in this experiment.

### Example 8

This example shows that HIN-2 expression is upregulated in the lung tissue of cystic fibrosis patients.

In this experiment, the expression of HIN-2 in the lung tissue of cystic fibrosis patients was compared to normal controls. Briefly, lung tissue sections from normal or cystic fibrosis patients were hybridized with antisense HIN-2 cDNA in an in situ hybridization experiment using standard conditions. The results showed that HIN-2 expression is upregulated in cystic fibrosis patients, as compared to the normal controls (data not shown).

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following claims.

1. An isolated protein comprising an amino acid sequence selected from the group consisting of:
a) an amino acid sequence selected from the group consisting of SEQ ID NO:2, amino acids 22-93 of SEQ ID NO:2, SEQ ID NO:4, and amino acids 22-91 of SEQ ID NO:4;
b) an amino acid sequence that is at least about 50% identical to said amino acid sequence of (a), wherein said protein has HIN-2 biological activity; and,
c) an amino acid sequence consisting of a fragment of an amino acid sequence of (a) that has HIN-2 biological activity.

2. (Cancelled)

3. (Cancelled)

4. (Cancelled)

5. A fusion protein comprising the isolated protein of claim 1 linked to a heterologous amino acid sequence.

6. A composition comprising the isolated protein of claim 1.

7. A human HIN-2 homologue, wherein said human HIN-2 homologue comprises an amino acid sequence that is at least about 50% identical to SEQ ID NO:2 or amino acids 22-93 of SEQ ID NO:2, and less than 100% identical to an amino acid sequence selected from the group consisting of SEQ ID NO:2, amino acids 22-93 of SEQ ID NO:2, SEQ ID NO:4, and amino acids 22-91 of SEQ ID NO:4;
wherein said human HIN-2 homologue is an agonist or antagonist of HIN-2 biological activity.

8. The human HIN-2 homologue of claim 7, wherein said HIN-2 homologue comprises an amino acid sequence that is less than about 97% identical to an amino acid sequence selected from the group consisting of SEQ ID NO:2, amino acids 22-93 of SEQ ID NO:2, SEQ ID NO:4, and amino acids 22-91 of SEQ ID NO:4.

9. The human HIN-2 homologue of claim 7, wherein said HIN-2 homologue comprises an amino acid sequence that is less than about 95% identical to an amino acid sequence selected from the group consisting of SEQ ID NO:2, amino acids 22-93 of SEQ ID NO:2, SEQ ID NO:4, and amino acids 22-91 of SEQ ID NO:4.

10. The human HIN-2 homologue of claim 7, wherein said HIN-2 homologue comprises an amino acid sequence that is less than about 90% identical to an amino acid sequence selected from the group consisting of SEQ ID NO:2, amino acids 22-93 of SEQ ID NO:2, SEQ ID NO:4, and amino acids 22-91 of SEQ ID NO:4.

11. The human HIN-2 homologue of claim 7, wherein said HIN-2 homologue comprises an amino acid sequence that is at least about 75% identical to SEQ ID NO:2 or amino acids 22-93 of SEQ ID NO:2.

12. The human HIN-2 homologue of claim 7, wherein said HIN-2 homologue comprises an amino acid sequence that is at least about 85% identical to SEQ ID NO:2 or amino acids 22-93 of SEQ ID NO:2.

13. The human HIN-2 homologue of claim 7, wherein said HIN-2 homologue comprises an amino acid sequence that is at least about 95% identical to SEQ ID NO:2 or amino acids 22-93 of SEQ ID NO:2.

14. The human HIN-2 homologue of wherein said human HIN-2 homologue is an agonist of HIN-2 biological activity.

15. The human HIN-2 homologue of claim 7, wherein said human HIN-2 homologue is an antagonist of HIN-2 biological activity.

16. The human HIN-2 homologue of claim 7, wherein said human HIN-2 homologue binds to a HIN-2 receptor.

17. The human HIN-2 homologue of claim 16, wherein said HIN-2 receptor is MARCO.

18. The human HIN-2 homologue of claim 7, wherein said human HIN-2 homologue binds to a bacterium or to a yeast.

19. The human HIN-2 homologue of claim 7, wherein said human HIN-2 homologue binds to lipopolysaccharide (LPS).

20. The human HIN-2 homologue of claim 7, wherein said human HIN-2 homologue binds to apolipoprotein A1.

21. (Cancelled)

22. (Cancelled)

23. (Cancelled)

24. (Cancelled)

25. (Cancelled)

26. A fusion protein comprising the human HIN-2 homologue of claim 7 that is linked to a heterologous amino acid sequence.

27. A composition comprising the human HIN-2 homologue of claim 7.

28. An isolated nucleic acid molecule comprising a nucleic acid sequence encoding the isolated protein of claim 1.

29. (Cancelled)

30. A recombinant nucleic acid molecule comprising the isolated nucleic acid molecule of claim 28, operatively linked to a transcription control sequence.

31. A recombinant host cell that has been transfected with the recombinant nucleic acid molecule of claim 30.

32. (Cancelled)

33. (Cancelled)

34. (Cancelled)

35. An isolated nucleic acid molecule comprising a nucleic acid sequence encoding the human HIN-2 homologue of claim 7.

36. A recombinant nucleic acid molecule comprising the isolated nucleic acid molecule of claim 35, operatively linked to a transcription control sequence.

37. A recombinant host cell that has been transfected with the recombinant nucleic acid molecule of claim 36.

38. (Cancelled)

39. (Cancelled)

40. (Cancelled)

41. An oligonucleotide consisting of at least 13 consecutive nucleotides of a nucleic acid sequence selected from the group consisting of SEQ ID NO:1 and SEQ ID NO:3.

42. (Cancelled)

43. (Cancelled)

44. (Cancelled)

45. (Cancelled)

46. (Cancelled)

47. An isolated binding agent selected from the group consisting of an antibody, an antigen binding fragment and a binding partner, wherein said binding agent selectively binds to an amino acid sequence selected from the group consisting of SEQ ID NO:2, amino acids 22-93 of SEQ ID NO:2, SEQ ID NO:4, and amino acids 22-91 of SEQ ID NO:4.

48. (Cancelled)

49. (Cancelled)

50. A method to identify a compound that regulates HIN-2 expression or biological activity, comprising:
a) contacting a HIN-2 protein, a biologically active fragment thereof, or a receptor-binding fragment thereof, with a putative regulatory compound; and
b) detecting whether said putative regulatory compound binds to or regulates the activity of said HIN-2 protein, biologically active fragment or receptor-binding fragment as compared to prior to contact with said compound;
wherein a compound that binds to said protein and increases or decreases activity of the protein, as compared to the protein in the absence of said compound, indicates that said putative regulatory compound is a regulator of HIN-2.

51. The method of claim 50, wherein said step of detecting comprises detecting whether HIN-2 expression or activity is regulated by contacting lung cells or lung tissue with said putative regulatory compound and measuring HIN-2 expression in said lung cells or lung tissue.

52. The method of claim 50, wherein said step of detecting comprises detecting whether HIN-2 expression or activity is regulated during allergic inflammation conditions in the presence of the putative regulatory compound as compared to in the absence of the putative regulatory compound.

53. The method of claim 50, wherein said step of detecting comprises detecting whether HIN-2 expression or activity is regulated during proinflammatory conditions in the presence of the putative regulatory compound as compared to in the absence of the putative regulatory compound.

54. The method of claim 50, wherein said step of detecting comprises detecting whether the putative regulatory compound inhibits, enhances, or competes with the binding of HIN-2 to a HIN-2 receptor.

55. The method of claim 54, wherein said HIN-2 receptor is MARCO.

56. The method of claim 50, wherein said step of detecting comprises detecting whether the putative regulatory compound inhibits, enhances, or competes with the binding of HIN-2 to a ligand selected from the group consisting of lipopolysaccharide (LPS), apolipoprotein AI, a bacterium and a yeast.

57. The method of claim 50, wherein said method further comprises a step of detecting whether the putative regulatory compound regulates inflammation in a cell, tissue, or non-human animal.

58. The method of claim 57, wherein said cells are lung cells or said tissue is lung tissue.

59. The method of claim 50, wherein the method further comprises a step of detecting whether the putative regulatory compound regulates the level of high density lipoproteins or low density lipoproteins in a mammal.

60. A method to identify a HIN-2 homologue that regulates HIN-2 biological activity, comprising detecting whether a putative HIN-2 homologue has at least one biological activity selected from the group consisting of:
binds to a HIN-2 receptor or to a HIN-2-binding portion of a HIN-2 receptor;
increases the activity of a HIN-2 receptor;
binds to a bacterial cell;
binds to a yeast cell;
binds to a lipopolysaccharide (LPS);
binds to apolipoprotein A1;
regulates phagocytosis of a bacterium or yeast by a macrophage as compared to in the absence of said putative HIN-2 homologue;
regulates lung inflammation as compared to in the absence of said putative HIN-2 homologue;
regulates airway hyperresponsiveness as compared to in the absence of said putative HIN-2 homologue; or
regulates innate immune responses in lung tissue as compared to in the absence of said putative HIN-2 homologue.

61. (Cancelled)

62. (Cancelled)

63. (Cancelled)

64. (Cancelled)

65. (Cancelled)

66. The method of claim 60, comprising contacting a HIN-2 receptor or a HIN-2-binding portion of a HIN-2 receptor with said putative HIN-2 homologue, and detecting whether said putative HIN-2 homologue binds to said HIN-2 receptor or HIN-2-binding portion.

67. The method of claim 60, wherein said HIN-2 receptor is MARCO.

68. A method to identify a compound that regulates the expression of Hin-2, comprising:
a) contacting a putative regulatory compound with a recombinant host cell that expresses a recombinant nucleic acid molecule encoding HIN-2 or a recombinant host cell that has been transfected with a nucleic acid sequence comprising a Hin-2 regulatory region operatively linked to a reporter nucleic acid sequence; and
b) detecting whether said putative regulatory compound regulates expression of said recombinant nucleic acid molecule encoding HIN-2 or said reporter nucleic acid sequence as compared to prior to contact with said compound;
wherein compounds that regulate expression of said recombinant nucleic acid molecule encoding HIN-2 or said reporter nucleic acid sequence, as compared to in the absence of said compound, indicates that said putative regulatory compound is a regulator of Hin-2 expression.

69. A method to diagnose a disorder associated with HIN-2 expression or biological activity, comprising detecting expression or biological activity of HIN-2 or a gene encoding HIN-2 in a tissue of a patient suspected of having said disorder, and comparing said expression or biological activity to a control, wherein a difference in the expression or biological activity of HIN-2 or a gene encoding HIN-2 in a tissue of the patient as compared to the control indicates a positive diagnosis of a disorder associated with HIN-2.

70. The method of claim 69, wherein said disorder is selected from the group consisting of: a lung disorder: a disorder associated with allergic inflammation; a disorder associated with microbial infection, and a lung cancer.

71. (Cancelled)

72. (Cancelled)

73. The method of claim 69, wherein said disorder is selected from the group consisting of asthma, interstitial lung disease, cystic fibrosis, rheumatoid arthritis, reactive arthritis, bacterial infection, yeast infection, and spondylarthropathy.

74. (Cancelled)

75. A composition comprising a portion of MARCO sufficient to bind to HIN-2 that is formulated for administration to lung tissue.

76. The composition of claim 75, wherein said MARCO is a soluble-receptor.

77. A method to regulate HIN-2 expression or activity, comprising administering to a patient a regulatory compound that regulates the biological activity of HIN-2.

78. The method of claim 77, wherein the compound is HIN-2 or a biologically active fragment thereof.

79. The method of claim 77, wherein said compound is a HIN-2 homologue.

80. The method of claim 79, wherein the HIN-2 homologue binds to MARCO and regulates phagocytosis of bacteria or yeast by a macrophage.

81. The method of claim 79, wherein the HIN-2 homologue binds to MARCO and induces NFκB activation in a cell expressing MARCO.

82. The method of claim 77, wherein said regulatory compound is an antibody, antigen binding fragment or binding partner that selectively binds to HIN-2.

83. The method of claim 77, wherein said regulatory compound is an antibody, an antigen binding fragment or a binding partner that selectively binds to a HIN-2 receptor.

84. The method of claim 83, wherein said HIN-2 receptor is MARCO.

85. The method of claim 77, wherein said regulatory compound is a HIN-2 receptor homologue that selectively binds to HIN-2.

86. The method of claim 85, wherein said HIN-2 receptor homologue is a soluble HIN-2 receptor.

87. The method of claim 85, wherein said HIN-2 receptor homologue is a homologue of MARCO.

88. The method of claim 77, wherein said regulatory compound is an isolated nucleic acid sequence that hybridizes to a nucleic acid sequence encoding at least 13 consecutive nucleotides of a gene encoding HIN-2.

89. The method of claim 77, wherein the regulatory compound regulates the ability of HIN-2 to bind to a HIN-2 receptor.

90. The method of claim 77, wherein the regulatory compound regulates the biological activity of HIN-2.

91. The method of claim 77, wherein the regulatory compound regulates the expression of HIN-2.

92. (Cancelled)

93. (Cancelled)

94. (Cancelled)

95. (Cancelled)

96. (Cancelled)

97. A method to regulate inflammation, comprising administering to a patient a compound that regulates the expression or biological activity of HIN-2 or a gene encoding HIN-2 in said patient.

98. (Cancelled)

99. (Cancelled)

100. (Cancelled)

101. (Cancelled)

102. A method to regulate the levels of high density lipoproteins or low density lipoproteins in a patient, comprising administering to a patient a regulatory compound that binds to HIN-2 and reduces binding of HIN-2 to MARCO or to apolipoprotein AI.

103. A method to treat cancer in a patient, wherein the tumor cells of said patient express MARCO, said method comprising administering to a patient with cancer a regulatory agent comprising a portion of HIN-2 sufficient to bind to MARCO which is linked to a therapeutic compound which reduces tumor cell growth or eliminates the tumor cell.

104. (Cancelled)

105. A method to deliver a therapeutic compound to a macrophage which expresses MARCO in a patient, comprising administering to the patient a regulatory agent comprising a portion of HIN-2 sufficient to bind to MARCO which is linked to said therapeutic compound.

106. A genetically modified non-human animal comprising a genetic modification within at least one allele of its Hin-2 locus, wherein the genetic modification results in a reduction of HIN-2 biological activity in the animal.

107. (Cancelled)

108. A method to detect a polymorphism or a loss of heterozygosity located in chromosome region 5q30-40, comprising contacting a nucleic acid molecule from a patient with an oligonucleotide comprising at least 13 consecutive nucleotides of SEQ ID NO:1.


SEQUENCE LISTING (8 sequences)

SEQ ID NO: 1 - DNA, 528 bp, Homo sapiens
  CDS (95)..(373)
gacactttgtatggcaagtggaaccactggcttggtggattttgctagatttttctgatttttaaactcctgaaaaatatcccagataactgtcatgaagctggtaactatcttcctgctggtgaccatcagcctttgtagttactctgctactgccttcctcatcaacaaagtgccccttcctgttgacaagttggcacctttacctctggacaacattcttccctttatggatccattaaagcttcttctgaaaactctgggcatttctgttgagcaccttgtggaggggctaaggaagtgtgtaaatgagctgggaccagaggcttctgaagctgtgaagaaactgctggaggcgctatcacacttggtgtgacatcaagataaagagcggaggtggatggggatggaagatgatgctcctatcctccctgcctgaaacctgttctaccaattatagatcaaatgccctaaaatgtagtgacccgtgaaaaggacaaataaagcaatgaatacataaaaaaaaaa

SEQ ID NO: 2 - protein, 93 aa, Homo sapiens
MKLVTIFLLVTISLCSYSATAFLINKVPLPVDKLAPLPLDNILPFMDPLKLLLKTLGISVEHLVEGLRKCVNELGPEASEAVKKLLEALSHLV

SEQ ID NO: 3 - DNA, 489 bp, Mus musculus
  CDS (84)..(356)
gcgagcagaactattgacaccgtgattttgttgggctttctgactgcattccaaagtcccggaaaacatcacaggcaccagctatgaagctggtatctatctttctgctggtgaccattggtatttgtggttattctgccactgcccttctcatcaaccgtctccctgttgttgacaaattacctgtacctttggacgacattattccctcatttgatcccttgaagatgcttctgaaaaccctgggcatttctgtagaacatctggtgacaggactgaagaagtgtgtggacgagctgggaccagaggcttccgaggccgtgaagaagcttctggaggctctttcacacctggtataaaatcttcataaagagatttaaggaaggagtatgaaaagaaggatttgctcactctggctggctggatctctcattctatcatttgtaaactgaatgtcccagagtttaaggagtctagaaaagtatgaat

SEQ ID NO: 4 - protein, 91 aa, Mus musculus
MKLVSIFLLVTIGICGYSATALLINRLPVVDKLPVPLDDIIPSFDPLKMLLKTLGISVEHLVTGLKKCVDELGPEASEAVKKLLEALSHLV

SEQ ID NO: 5 - DNA, 1863 bp, Homo sapiens
  CDS (137)..(1699)
gggggccaaagggaagtgctgcgaggtttacaaccagctgcagtggttcgatgggaaggatctttctccaagtggttcctcttgaggggagcatttctgctggctccaggactttggccatctataaagcttggcaatgagaaataagaaaattctcaaggaggacgagctcttgagtgagacccaacaagctgcttttcaccaaattgcaatggagcctttcgaaatcaatgttccaaagcccaagaggagaaatggggtgaacttctccctagctgtggtggtcatctacctgatcctgctcaccgctggcgctgggctgctggtggtccaagttctgaatctgcaggcgcggctccgggtcctggagatgtatttcctcaatgacactctggcggctgaggacagcccgtccttctccttgctgcagtcagcacaccctggagaacacctggctcagggtgcatcgaggctgcaagtcctgcaggcccaactcacctgggtccgcgtcagccatgagcacttgctgcagcgggtagacaacttcactcagaacccagggatgttcagaatcaaaggtgaacaaggcgccccaggtcttcaaggccacaagggggccatgggcatgcctggtgcccctggcccgccgggaccacctgctgagaagggagccaagggggctatgggacgagatggagcaacaggcccctcgggaccccaaggcccaccgggagtcaagggagaggcgggcctccaaggaccccagggtgctccagggaagcaaggagccactggcaccccaggaccccaaggagagaagggcagcaaaggcgatgggggtctcattggcccaaaaggggaaactggaactaagggagagaaaggagacctgggtctcccaggaagcaaaggggacaggggcatgaaaggagatgcaggggtcatggggcctcctggagcccaggggagtaaaggtgacttcgggaggccaggcccaccaggtttggctggttttcctggagctaaaggagatcaaggacaacctggactgcagggtgttccgggccctcctggtgcagtgggacacccaggtgccaagggtgagcctggcagtgctggctcccctgggcgagcaggacttccagggagccccgggagtccaggagccacaggcctgaaaggaagcaaaggggacacaggacttcaaggacagcaaggaagaaaaggagaatcaggagttccaggccctgcaggtgtgaagggagaacaggggagcccagggctggcaggtcccaagggagcccctggacaagctggccagaagggagaccagggagtgaaaggatcttctggggagcaaggagtaaagggagaaaaaggtgaaagaggtgaaaactcagtgtccgtcaggattgtcggcagtagtaaccgaggccgggctgaagtttactacagtggtacctgggggacaatttgcgatgacgagtggcaaaattctgatgccattgtcttctgccgcatgctgggttactccaaaggaagggccctgtacaaagtgggagctggcactgggcagatctggctggataatgttcagtgtcggggcacggagagtaccctgtggagctgcaccaagaatagctggggccatcatgactgcagccacgaggaggacgcaggcgtggagtgcagcgtctgacccggaaaccctttcacttctctgctcccgaggtgtcctcgggctcatatgtgggaaggcagaggatctctgaggagttccctggggacaactgagcagcctctggagaggggccattaataaagctcaacatcaaaaaaaaaaaagaaaaaaaaaaaaaaaaa

SEQ ID NO: 6 - protein, 520 aa, Homo sapiens
MRNKKILKEDELLSETQQAAFHQIAMEPFEINVPKPKRRNGVNFSLAVVVIYLILLTAGAGLLVVQVLNLQARLRVLEMYFLNDTLAAEDSPSFSLLQSAHPGEHLAQGASRLQVLQAQLTWVRVSHEHLLQRVDNFTQNPGMFRIKGEQGAPGLQGHKGAMGMPGAPGPPGPPAEKGAKGAMGRDGATGPSGPQGPPGVKGEAGLQGPQGAPGKQGATGTPGPQGEKGSKGDGGLIGPKGETGTKGEKGDLGLPGSKGDRGMKGDAGVMGPPGAQGSKGDFGRPGPPGLAGFPGAKGDQGQPGLQGVPGPPGAVGHPGAKGEPGSAGSPGRAGLPGSPGSPGATGLKGSKGDTGLQGQQGRKGESGVPGPAGVKGEQGSPGLAGPKGAPGQAGQKGDQGVKGSSGEQGVKGEKGERGENSVSVRIVGSSNRGRAEVYYSGTWGTICDDEWQNSDAIVFCRMLGYSKGRALYKVGAGTGQIWLDNVQCRGTESTLWSCTKNSWGHHDCSHEEDAGVECSV

SEQ ID NO: 7 - DNA, 1984 bp, Mus musculus
  CDS (308)..(1864)
cagtgtcacctaagggcctggggacatggagggtctttcccaaacttcttcaattccccataacaggcagcttccccacacaacagtttcacatgtgaacagcctgatatttaagcaaaaatcagttctacactaaggagcagccgggtccccacagccaggaaacattgtgcaaattgaaaaatcattgccaaagggaagttgtatgcatctccagctagctgccgcagttaaatgggagccctgcttcctcctaggggagagtttctgctggctccagggctttggccacctataaagcttagcaatgggaagtaaagaactcctcaaagaggaagacttcttgggcagcacagaagacagagccgattttgaccaagctatgttccctgtgatggagaccttcgaaatcaatgatccagtgcccaagaagagaaatggggggaccttctgcatggcagtcatggccatccacctgatcctgctcacggcaggtactgcactgctgctgattcaagttctcaatctgcaggagcagctccagatgctagagatgtgctgtggcaatggatcactagctatcgaggacaagcccttcttctcgctgcagtgggcacccaaaacacacctggtacctagagcacaggggctgcaagccttgcaggcccagctcagctgggtccataccagccaggagcaactccgtcagcagttcaacaacctcactcaaaatccagagttgttccagattaaaggtgaacgaggctctccaggtccaaaaggggccccgggtgctcctggaatccccgggctgcctgggccagctgctgagaagggagaaaagggggctgcaggtcgtgatggaaccccaggtgtccaaggaccccagggcccaccaggcagcaagggagaggcaggcctccagggacttacgggtgcaccagggaagcaaggagcaactggtgctccaggacctcgaggagagaagggcagcaaaggtgacataggtctcactggccccaagggggaacatggcaccaagggagacaaaggggacctaggccttccaggaaacaaaggggacatgggcatgaagggagacacggggcccatggggtcccctggagctcagggaggtaaaggtgatgctggaaaaccaggcctaccaggtttggctggatctccaggagtcaaaggtgaccaaggaaaacctggagtgcagggtgttccaggccctcaaggtgcaccaggactttcaggtgccaagggtgagccaggacgcactggtcttcctgggccagcaggacccccgggaattgctgggaatccagggattgcaggtgtgaaaggaagcaagggtgacacaggaattcaaggacagaaaggcacaaaaggagaatcaggagtcccaggtcttgtaggcagaaagggagacactggaagccctgggctggcaggtcccaaaggagaacctggacgagtcggtcagaagggagacccggggatgaaagggtcttctggccagcaaggacaaaagggagaaaagggtcaaaaaggcgaatctttccaacgcgtccggatcatgggtggcaccaacagaggccgagctgaagtttactataacaatgagtgggggacaatttgtgatgatgattgggataataatgatgcgactgtcttctgtcgcatgctcggttactccagagggagagcacttagcagctatggaggtggctctgggaacatctggctggacaatgtgaattgtcggggcacagagaacagtttgtgggactgcagtaagaactcctggggcaatcacaattgcgtacataatgaagatgcgggtgtggaatgctcctgacttgggagcccgagaggtcatcagtgtgtccccaggtgtctttggttccacccacatggaaatctgtgggcttgccaactctgttgaggggaagttaataaagctcaagtggggatctga

SEQ ID NO: 8 - protein, 518 aa, Mus musculus
MGSKELLKEEDFLGSTEDRADFDQAMFPVMETFEINDPVPKKRNGGTFCMAVMAIHLILLTAGTALLLIQVLNLQEQLQMLEMCCGNGSLAIEDKPFFSLQWAPKTHLVPRAQGLQALQAQLSWVHTSQEQLRQQFNNLTQNPELFQIKGERGSPGPKGAPGAPGIPGLPGPAAEKGEKGAAGRDGTPGVQGPQGPPGSKGEAGLQGLTGAPGKQGATGAPGPRGEKGSKGDIGLTGPKGEHGTKGDKGDLGLPGNKGDMGMKGDTGPMGSPGAQGGKGDAGKPGLPGLAGSPGVKGDQGKPGVQGVPGPQGAPGLSGAKGEPGRTGLPGPAGPPGIAGNPGIAGVKGSKGDTGIQGQKGTKGESGVPGLVGRKGDTGSPGLAGPKGEPGRVGQKGDPGMKGSSGQQGQKGEKGQKGESFQRVRIMGGTNRGRAEVYYNNEWGTICDDDWDNNDATVFCRMLGYSRGRALSSYGGGSGNIWLDNVNCRGTENSLWDCSKNSWGNHNCVHNEDAGVECS